(19) United States
(12) Patent Application Publication
(10) Pub. No.: US 2022/0328140 A1
(43) Pub. Date: Oct. 13, 2022

(54) METHODS AND APPARATUS FOR THERAPEUTIC FEASIBILITY ASSESSMENT USING QUANTITATIVE SYSTEMS PHARMACOLOGY AND RULE-BASED REASONING SYSTEMS

(71) Applicant: Applied BioMath, LLC, Concord, MA (US)

(72) Inventors: Joshua Farley APGAR, Lexington, MA (US); John M. BURKE, Jr., Acton, MA (US); David Christopher FLOWERS, Arlington, MA (US); David Robert HAGEN, Billerica, MA (US); Andrew Lewis MATTESON, Ithaca, NY (US); James Choate SCHAFF, Groton, MA (US)

(73) Assignee: Applied BioMath, LLC, Concord, MA (US)

(21) Appl. No.: 17/713,942

(22) Filed: Apr. 5, 2022

Related U.S. Application Data
(60) Provisional application No. 63/171,017, filed on Apr. 5, 2021.

(51) Int. Cl. G16C 20/50; G16C 20/30; G16C 20/80; G16C 20/70; G16C 20/10

(52) U.S. Cl. CPC G16C 20/50; G16C 20/30; G16C 20/80; G16C 20/70; G16C 20/10

(57) ABSTRACT

In some embodiments, the early feasibility assessment (EFA) system generates a set of quantitative systems pharmacology (QSP) models using rule-based reasoning systems to efficiently assess the feasibility of therapeutic drug candidates. With each QSP model, the EFA system provides sets of parameters for the user to easily contrast and compare various scenarios to explore risk and uncertainty of developing the therapeutic drug candidate, while high-performance computing provides near real-time simulation of the scenarios. The EFA system performs 1-dimensional or 2-dimensional scans of parameters and output feasibility criteria including, for example, maximum inhibition, activation, and target engagement. The assessment results and key parameters values can be presented via a user interface of the EFA system which allows user interactions with, for example, dose-response and pharmacokinetic and pharmacodynamic (PK/PD) plots.

### CROSS-REFERENCE TO RELATED APPLICATION

This application claims priority to and the benefit of U.S. Provisional Patent Application No. 63/171,017, filed Apr. 5, 2021, and entitled “METHOD AND APPARATUS FOR THERAPEUTIC FEASIBILITY ASSESSMENT USING QUANTITATIVE SYSTEMS PHARMACOLOGY,” the contents of which are incorporated herein by reference in its entirety.

### TECHNICAL FIELD

Some embodiments described herein relate generally to quantitative systems pharmacology and generation of rule-based reasoning models to efficiently assess the feasibility of drug candidates. In particular, but not by way of limitation, some embodiments described herein relate to methods and apparatus for therapeutic feasibility assessment using quantitative systems pharmacology and artificial intelligence methods including, for example, knowledge processing systems where a rule-based reasoning model is generated and then applied to a collection of facts and relationships.

### BACKGROUND

Quantitative systems pharmacology (QSP) uses mathematical computer models to characterize biological systems, disease processes, and drug pharmacology for drug discovery and development. QSP can examine interactions between a therapeutic drug candidate and a target biological system and help guide appropriate study design in the decision-making process to discover and develop the right drug for a disease. Some known QSP methods, however, require extensive human and computing resources to develop and iteratively optimize the QSP models. There is often no experimental data or it takes an extended amount of time and money that may delay or impair clinical studies of the drug candidates.

Accordingly, a need exists for generation of a rule-based reasoning model that uses logic processing to allow researchers to efficiently assess the feasibility of drug candidates, explore and optimize key factors in the drug development process for better clinical outcomes.

### SUMMARY

In some embodiments, the early feasibility assessment (EFA) system generates a set of quantitative systems pharmacology (QSP) models using rule-based reasoning systems to efficiently assess the feasibility of therapeutic drug candidates. With each QSP model, the EFA system provides sets of parameters for the user to easily contrast and compare various scenarios to explore risk and uncertainty of developing the therapeutic drug candidate, while high-performance computing provides near real-time simulation of the scenarios. The EFA system performs 1-dimensional or 2-dimensional scans of parameters and output feasibility criteria including, for example, maximum inhibition, activation, and target engagement. The assessment results and key parameters values can be presented via a user interface of the EFA system which allows user interactions with, for example, dose-response and pharmacokinetic and pharmacodynamic (PK/PD) plots.

### BRIEF DESCRIPTION OF THE DRAWINGS

FIG. 1 illustrates an example schematic diagram illustrating a system of information exchange between a server and a client device (e.g., an electronic device), according to some embodiments.

FIG. 2 illustrates a flow chart of a process for early feasibility assessment (EFA), according to some embodiments.

FIG. 3 illustrates an example user interface of the EFA, according to some embodiments.

FIGS. 4A-4C illustrate example user interfaces of the EFA, according to some embodiments.

FIGS. 5A-5D illustrate example user interfaces of the EFA, according to some embodiments.

FIG. 6 illustrates an example user interface of the EFA, according to some embodiments.

FIG. 7 illustrates an example user interface of the EFA, according to some embodiments.

FIG. 8 illustrates an example user interface of the EFA, according to some embodiments.

FIGS. 9A-9F illustrate example user interfaces of the EFA, according to some embodiments.

FIGS. 10A-10B illustrate example user interfaces of the EFA, according to some embodiments.

FIGS. 11A-11C illustrate example results of the EFA, according to some embodiments.

FIGS. 12A-12B show schematic diagrams illustrating a biotherapeutic scenario of a bispecific anti-ligand x anti-ligand example model, according to some embodiments.

FIGS. 13A-13B show schematic diagrams illustrating a biotherapeutic scenario of an example bispecific anti-ligand x anti-receptor model, according to some embodiments.

FIGS. 14A-14B show schematic diagrams illustrating a biotherapeutic scenario of an example monospecific anti-ligand model, according to some embodiments.

FIGS. 15A-15B show schematic diagrams illustrating a biotherapeutic scenario of an example bispecific anti-receptor x anti-receptor model, according to some embodiments.

FIGS. 16A-16B show schematic diagrams illustrating a biotherapeutic scenario of an example monospecific anti-receptor model, according to some embodiments.

FIGS. 17A-17B show schematic diagrams illustrating a biotherapeutic scenario of an example T cell engager for solid tumors model, according to some embodiments.

FIG. 18 shows a diagram illustrating connectivity between compartments for example four-compartment QSP models, according to some embodiments.

### DETAILED DESCRIPTION

Early drug research and development poses many questions when determining if a therapeutic drug enters the portfolio (of, for example, clinical studies), and how difficult (from a discovery and engineering perspective) it will be to develop. Embodiments described herein include an early feasibility assessment (EFA) system including generation of rule-based reasoning models and an interactive application that helps researchers quickly assess the feasibility of drug candidates including their therapeutic characteristics (e.g. half life, affinity) that are used to achieve target criteria (e.g., ligand binding, receptor inhibition) as a function of, for example, biological characteristics (e.g., ligand concentration and ligand half life), therapeutic characteristics (e.g., mode of action, molecular weight, format), and posology or dosing characteristics (e.g., dose amount, dose frequency).

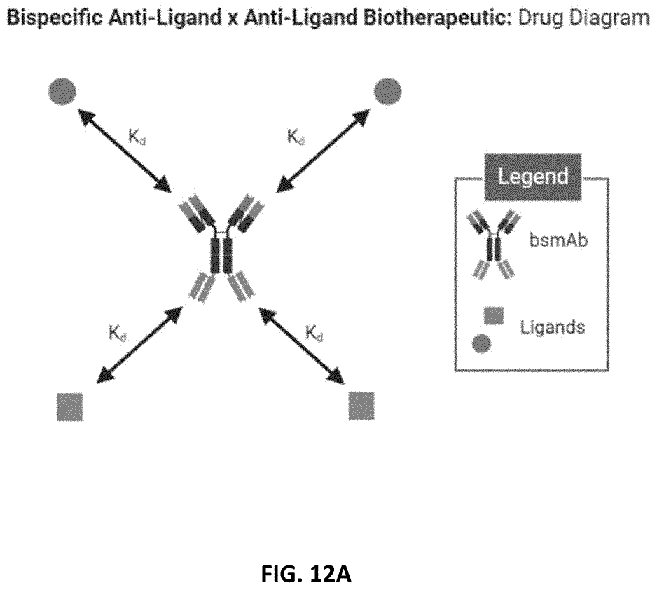

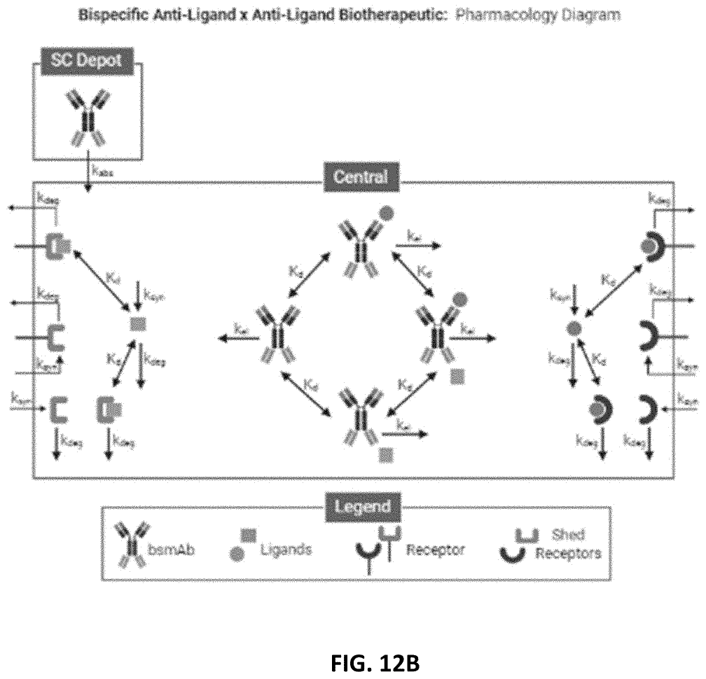

In some embodiments, the EFA system generates a set of models (as described below with respect to FIGS. 12A-18) that users can select. The set of models can include, but are not limited to, quantitative systems pharmacology (QSP) models, mechanistic models, semi-mechanistic models, pharmacokinetic (PK) models, pharmacokinetic and dynamic (PK/PD) models, fit for purpose models, systems biology models, and physiologic based pharmacokinetic models (PBPK). The systems and methods described herein are described using QSP models, but other types of models described above can be used instead of or in addition to QSP models. With each model, the EFA system provides sets of parameters for the user to easily contrast and compare various scenarios to explore risk and uncertainty of a therapeutic drug candidate, while high-performance computing provides near real-time simulation of the scenarios. The EFA system performs 1-dimensional or 2-dimensional scans, samplings, searches, and/or optimizations of parameters and outputs feasibility criteria including, for example, inhibition, activation, and target engagement. The assessment results and key parameter values can be presented via a user interface of the EFA system, which allows user interactions with, for example, dose-response and mechanistic pharmacokinetic and pharmacodynamic (PK/PD) plots.

In some embodiments, the EFA system provides QSP models and analysis that have been designed to be right sized for answering scientific questions in the early drug discovery and development process (e.g., at the start of a project, right before it may enter into the portfolio and being assigned significant resources and budget). The EFA system allows researchers to, for example, identify what drug parameters are desired and/or required, identify what biological parameters are important, de-risk a project by highlighting challenges early-on, and determine what experiments are important. The EFA system can be used to provide decision-making (e.g., go/no-go) guidance, optimize screening funnels for, for example, lead generation (a stage in early drug discovery where small molecule hits from a high throughput screen are evaluated and undergo optimization and/or improvement to identify promising lead compounds), prioritize key experiments/data, assess risk earlier, align stakeholders earlier, manage resources, and/or the like. Other advantages of the EFA system include easy manipulations by the user, the user not needing to know the biology details, a large number of parameter spaces that can be studied, and low latency/near real-time analyses of the QSP models.

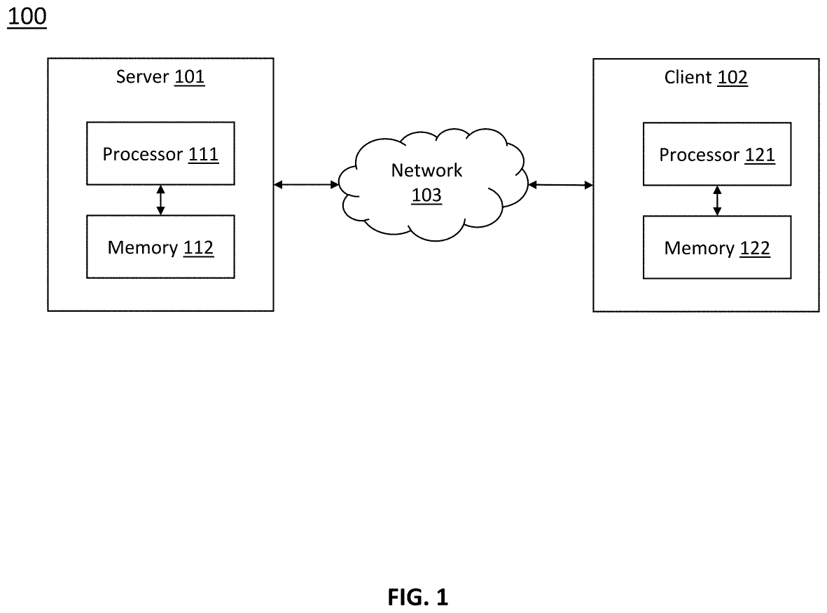

FIG. 1 illustrates an example schematic diagram illustrating the early feasibility assessment (EFA) system 100, according to some embodiments. In some embodiments, the EFA system 100 includes a first compute device such as a server 101 and a second compute device such as a client device 102 configured to communicate with the server 101 via a network 103.

The server 101 can be a compute device (or multiple compute devices) having a processor 111 and a memory 112 operatively coupled to the processor 111. In some instances, the server 101 can be any combination of hardware-based modules (e.g., a field-programmable gate array (FPGA), an application specific integrated circuit (ASIC), a digital signal processor (DSP), a graphics processing unit (GPU)) and/or software-based modules (computer code stored in memory 112 and/or executed at the processor 111) capable of performing one or more specific functions associated with that module. In some instances, the server 101 can be a server such as, for example, a web server, an application server, a proxy server, a telnet server, a file transfer protocol (FTP) server, a mail server, a list server, a collaboration server and/or the like. In some instances, the server 101 can be a personal computing device such as a desktop computer, a laptop computer, a personal digital assistant (PDA), a standard mobile telephone, a tablet personal computer (PC), and/or so forth.

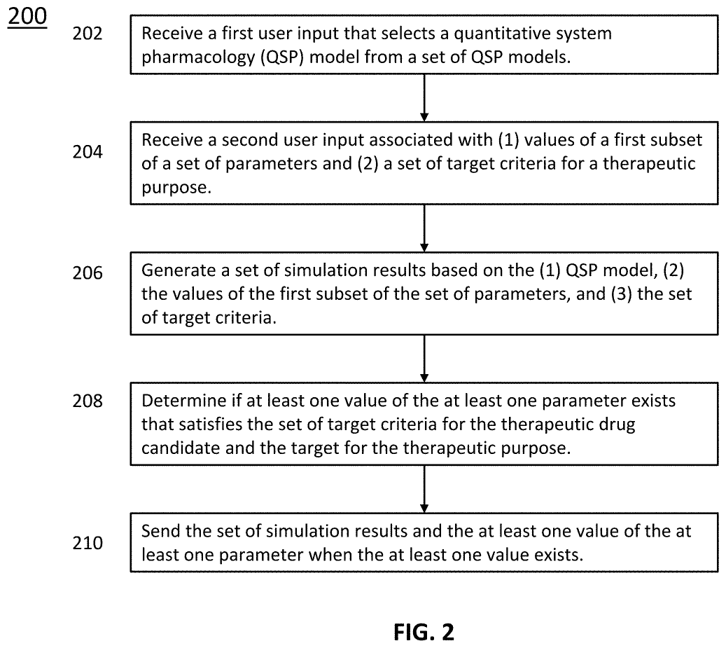

The memory 112 can be, for example, a random-access memory (RAM) (e.g., a dynamic RAM, a static RAM), a flash memory, a removable memory, a hard drive, a database and/or so forth. In some implementations, the memory 112 can include (or store), for example, a database, process, application, virtual machine, and/or other software modules (stored and/or executing in hardware) and/or hardware modules configured to execute an EFA process(es) as described with regards to FIG. 2. In such implementations, instructions for executing the EFA process(es) and/or the associated methods can be stored within the memory 112 and executed at the processor 111. In some implementations, the memory 112 can store QSP models, experimental data, past assessment results, and/or the like.

The processor 111 can be configured to, for example, write data into and read data from the memory 112, and execute the instructions stored within the memory 112. The processor 111 can also be configured to execute and/or control, for example, the operations of other components of the server 101 (such as a network interface card, other peripheral processing components (not shown)). In some implementations, based on the instructions stored within the memory 112, the processor 111 can be configured to execute one or more steps of the EFA process(es) as described with regards to FIG. 2.

The client device 102 can be a compute device having a processor 121 and a memory 122 operatively coupled to the processor 121. In some instances, the client device 102 can be a mobile device, a tablet personal computer, a personal computing device, a desktop computer, a laptop computer, and/or the like. The client device 102 can be any combination of hardware-based modules (e.g., a field-programmable gate array (FPGA), an application specific integrated circuit (ASIC), a digital signal processor (DSP)) and/or software-based modules (computer code stored in memory 122 and/or executed at the processor 121) capable of performing one or more specific functions associated with that module.

The memory 122 can be, for example, a random-access memory (RAM) (e.g., a dynamic RAM, a static RAM), a flash memory, a removable memory, a hard drive, a database and/or so forth. In some implementations, the memory 122 can include (or store), for example, a database, process, application, virtual machine, and/or other software modules (stored and/or executing in hardware) and/or hardware modules configured to execute an EFA process(es) as described with regards to FIG. 2. In such implementations, instructions for executing the EFA process(es) as described with regards to FIG. 2 and/or the associated methods can be stored within the memory 122 and executed at the processor 121. In some implementations, the memory 122 can store QSP models, experimental data, past assessment results, and/or the like.

The processor 121 can be configured to, for example, write data into and read data from the memory 122, and execute the instructions stored within the memory 122. The processor 121 can also be configured to execute and/or control, for example, the operations of other components of the client 102 (such as a network interface card, other peripheral processing components (not shown)). In some implementations, based on the instructions stored within the memory 122, the processor 121 can be configured to execute one or more steps of the EFA process(es) as described with regards to FIG. 2. In some implementations, the processor 121 and the processor 111 can be collectively configured to execute the EFA process(es) as described with regards to FIG. 2.

In some embodiments, the client device 102 can be configured with a user interface, e.g., a graphical user interface. The client device 102 can be an electronic device that can communicate the EFA application (or instructions stored in the memory 112 and executed by the processor 111) via the user interface (e.g., a web browser, or a mobile application that presents the user interface to a user).

In some implementations, the server 101 can be configured to provide the user interface capabilities as well as the computation capabilities (or computational resources) using the QSP models to the client device 102 via the network 103 (e.g., the client device 102 can act as a thin client). In some implementations, the server 101 can be configured to provide the user interface capabilities to the client device 102 via the network 103. The server 101 can be communicatively coupled to additional computing devices (including processors and memories; not shown in FIG. 1), which provide the computation capabilities using the QSP models to the client device 102. In these implementations, the client device 102 can request (or call) the EFA application (e.g., instructions stored in the memory 112 and executed by the processor 111 and/or computing devices that provide the computation capabilities) through an Application Programming Interface (API), such as Representational state transfer (REST), GraphQL, Simple Object Access Protocol (SOAP), Remote Procedure Call (RPC), and/or the like. The EFA application (e.g., instructions stored in the memory 112 and executed by the processor 111 and/or computing devices that provide the computation capabilities), in some implementations, can be requested or called through directly managing requests to the protocol (for instance HTTP, TCP, or RPC calls), or may be managed through a client library or a software development kit (SDK) installed on the client device 102. The client library can support access from scientific applications or integrated development environments (IDEs) like MATLAB, RStudio, JupyterHub, and/or others. Clients and SDKs can be incorporated in “macro” programming languages such as Visual Basic for Applications (VBA) for integration with spreadsheet environments like Google Sheets or Microsoft Excel. In some implementations, the interactive graphical user interface can be called from a scripting environment or through an application programming interface.

In some embodiments, the EFA application can be called from (or be part of) another compiled or interpreted application, or from a scientific programming environment such as a script, desktop application, IDE, a serverless cloud application, Python/Scipy, R, MATLAB, and/or the like.

In some embodiments, the computational capabilities (or computational resources) provided by the server 101 (or in some implementations, computing devices communicatively coupled to the server 101) can be accessed by the client device 102 over a network, intranet, internet, extranet, peer-to-peer connection, or other collection of networked computer systems. The computational resources may run on machines, virtual machines, containers, or other abstractions of computer hardware.

In some embodiments, the server 101 can be configured to provide the EFA application via a remote cloud computing server(s) (not shown). For example, the client device 102 can access the EFA application via a user interface (e.g., a web browser) installed on the client device 102. The server 101 can be configured to store instructions of the EFA application in the memory 112 and the EFA application is executed by the processor 111. The EFA application can communicate with a remote cloud computing server(s) (not shown) to perform analysis with QSP models using the computational resources residing on the remote cloud computing server(s). The results of computation can be sent to the EFA application hosted by the server 101 and presented to the user interface installed on the client device 102.

In some implementations, load balancers (or proxies, or reverse proxies) of the server 101 (or computing devices that provide the computational resources) can distribute requests received from the client device 102. The server 101 can leverage a job queue that coordinates between several computing devices (or computational resources). The server 101 (or the computing devices communicatively coupled to the server 101) can communicate with the job queue using a queuing protocol, such as Kafka, ActiveMQ, AMQP, ZeroMQ, Amazon SNS, and/or the like. In some implementations, the server 101 (or the computing devices communicatively coupled to the server 101) may send the results of the EFA process through, for example, NoSQL or SQL databases, such as CouchDB, MongoDB, Redis, PostGreSQL, MySQL, SQLITE, Oracle, or the like. In some implementations, the server 101 (or the computing devices communicatively coupled to the server 101) may send the results of the EFA process through queueing systems. In some implementations, the server 101 (or the computing devices communicatively coupled to the server 101) may send the results of EFA process through sockets.

“Workers” are abstractions of computing devices (or computational resources on the server 101) that perform one or more tasks (or computational capabilities of the EFA application) in response to a function call, a message in a queue, a request by a client device 102, a request by an application, or other signal that computes work is needed. Workers are pieces of software that may run in containers, virtual machines, physical computers, “serverless” cloud tools, or other abstractions of computing environments.

Workers may communicate the results of computation through NoSQL or SQL databases, such as CouchDB, MongoDB, Redis, PostGreSQL, MySQL, SQLITE, or Oracle. For some applications, workers may communicate the results of jobs through queueing systems. Other applications require workers to communicate results through sockets. Workers may write results of a job to a local or remote hard disk, SSD, tape drives, computer memory, flash memory or other storage media for digital data.

Workers may run on physical machines, or logical machines such as virtual machines, containers, or orchestrated containers. One class of workers includes, for example, “serverless” deployments in which application code describing computation is run as part of a cloud hosted service such as AWS Lambda or AWS Fargate.

The communication of workers can be mediated by data structures and algorithms forming a task queue system that optimizes and/or improves the time to execute a job after the job reaches the queue while still enabling high levels of throughput. The EFA system can provide orders of magnitude faster performance over general purpose queuing systems commonly used (for example “Celery”). The EFA system can enable a distributed queuing system to support both interactive and non-interactive workloads.

In some implementations, the functionality of the server 101 and the client 102 are collocated on a single computing device. In some implementations, the server 101 and the client 102 reside on different computing devices and communicate with each other over the network 103.

FIG. 2 illustrates a flow chart of an example early feasibility assessment (EFA) process 200, according to some embodiments. This EFA process 200 can be implemented at a processor and/or a memory (e.g., processor 111 or memory 112 at the server 101 as discussed with respect to FIG. 1, the processor 121 or memory 122 at the client 102 as described with respect to FIG. 1, and/or the processor or memory at computing devices that are operatively or communicatively coupled to the server 101 discussed with respect to FIG. 1).

At step 202, the EFA process 200 includes receiving a first user input that selects a model from a set of models. The set of models can include, but is not limited to, quantitative systems pharmacology (QSP) models, mechanistic models, semi-mechanistic models, pharmacokinetic (PK) models, pharmacokinetic and dynamic (PK/PD) models, fit for purpose models, systems biology models, and/or physiologic based pharmacokinetic models (PBPK). While described herein as using QSP models (but not limited to QSP models), other types of models described above can be used instead of or in addition to QSP models. Example models and example mechanisms that can be used to construct the example models that can be used in the EFA process by the EFA system are shown and described below.

The EFA process 200 includes providing or presenting (e.g., via an application or a user interface on a client device) a library of single- and multi-compartment QSP models (as described below with respect to FIGS. 12A-18) that includes a variety of pharmacologies (or characterizations of biological interactions between therapeutic drug candidates and targets). The EFA process 200 includes providing or presenting descriptions of each QSP model including, for example, a model diagram or other model documentation including information about the model parameters. The server (e.g., the server 101 as discussed with respect to FIG. 1) may receive a user input from the client device (e.g., the client device 102 as discussed with respect to FIG. 1) via a user interface at the client device that selects a QSP model. The QSP model includes a set of parameters and includes characterizations of biological interactions between a therapeutic drug candidate and a target. In some implementations, instead of selecting a QSP model from the library of the QSP models, the user can provide a model to the server or the EFA system.

At step 204, this EFA process 200 includes receiving a second user input. The second user input is associated with values of a first subset of the set of parameters and a set of target criteria for the therapeutic purpose. In some implementations, the first subset of the set of parameters includes a nominal macro parameter from a set of nominal macro parameters, which includes at least one of a time constant, a concentration, or a thermodynamic constant.

In some implementations, the set of parameters includes, but is not limited to, the interval between doses of the drug, the amount of each dose, the affinity of the drug, the number of doses to be given, the molecular weight of the drug, the first-order elimination rate of the drug, the first-order absorption rate of the drug, the body weight of the subject, and/or the like.

In some implementations, the server can provide a set of target criteria including, for example, percent inhibition, activation, target engagement, therapeutic index, concentrations (or more or more states of the model) or amounts of states in the model. The target criteria may also include any formula of or value associated with the above including min over time, max over time, value at a particular time, value at the end of the dose interval, average value or the integral over time of any specific time interval or over all time, and/or the like. In some implementations, the target criteria of inhibition can be measured as inhibition of ligand receptor complex. The target criteria of inhibition can be measured as a percent of untreated. The target engagement can be measured as percent reduction of free target due to drug binding. The target engagement can be measured as a percent of untreated. The target criteria of activation can be measured as percent of receptor occupied by ligand or drug. The target criteria of activation can be measured as a percent of total (e.g., used for agonists). The set of parameters can be different for different QSP models.

At step 206, this EFA process 200 includes generating a set of simulation results based on the (1) QSP model, (2) the values of the first subset of the set of parameters, and (3) the set of target criteria. The generating the set of simulation results includes scanning, from a first value of a set of values to a second value from the set of values, at least one parameter from a second subset of the set of parameters. In some implementations, the EFA process 200 includes performing a search (or a scan) for at least one parameter value. The EFA process includes scanning the parameter value between a range of values and finding the minimum and/or maximum values (or other critical values) of the search/scan parameter such that all criteria value expressions are greater than or equal to the associated criteria target value. If no such minimum/maximum exists between the limits (or range), then the EFA process includes generating an output to indicate any information regarding why the values cannot be found. Additional details of generating the set of simulation results based on the QSP model, the first subset of the set of parameters, and the set of target criteria are discussed in the “Methods” section below.

In some implementations, the EFA process includes performing a parameter scan over a model parameter (or one-dimensional parameter scan), and reporting the value of the model criteria at multiple parameter values. The EFA process includes generating a grid, sample, or collection of values from the lowest value to the highest value, with either log spacing, linear spacing, logit spacing, probit spacing, random spacing, quasi-random spacing, or the like depending on the user selection. The EFA process includes simulating using the model with the named parameter set to each value in the grid, and the rest of the values set to the user supplied nominal parameters, or model supplied parameters. The EFA process includes searching, optimizing, interpolating, solving, or scanning for the largest parameter value, the smallest parameter value, or a range of or multiple ranges of parameter values that imply a criteria value or collection of criteria values are above/below their target values depending on user selection.

In some implementations, the EFA process includes performing a parameter scan, sampling, or optimization over two model parameters (or two-dimensional parameter scan), and generating a report having the value of user supplied expressions at multiple parameter values. The EFA process includes generating a 2D grid, sample, or other collection of values—one dimension for each parameter. The EFA process includes simulating using the model with the named parameters set to their respective values in the 2D grid, sample or other collection, and the rest of the values set to the user supplied nominal parameters.

In some implementations, the EFA process defines a function or algebraic system that relates one or two input parameters with more than two parameters. The EFA process includes performing a parameter scan, sampling, or optimization over the input parameters and simulating the model with the output parameters.

In some implementations, the EFA process 200 includes retrieving values of a third subset of the set of parameters from a data storage, public database, literature, results of an on- or offline calculation of any of the above, and/or the like. Generating the set of simulation results includes simulation results based on the values of the first, second, and third subset of the set of parameters. In other words, the EFA process 200 allows the user to define a first subset of parameters from the set of parameters associated with a selected model, and the EFA process 200 includes scanning a second subset of parameters from a minimum value to a maximum value to find critical values that meet the target criteria, and to map out the space between. In addition, the EFA process 200 includes setting a third subset of parameters from the set of parameters to values that are based on information retrieved from a data storage, public database, literature, results of an on- or offline calculation of any of the above, and/or the like.

In some implementations, generating the set of simulation results includes generating a set of partial factorial expansion of a high dimensional parameter space. In some implementations, generating the set of simulation results includes adjusting the high dimensional parameter space by decimating un-needed dimensions. In some implementations, generating the set of simulation results is in response to the first user input and the second user input.

At step 208, the EFA process 200 includes determining if at least one value of the at least one parameter exists that satisfies the set of target criteria for the therapeutic drug candidate and the target for the therapeutic purpose. In some implementations, determining that at least one value of the at least one parameter exists includes using a binary search, bisection search, Newton's method, variable order interpolation, or other process to calculate or approximate solutions to the satisfaction problem. In other words, the EFA process uses efficient algorithms to calculate or approximate solutions (i.e., if at least one value of the at least one parameter exists that satisfies the set of target criteria). To accelerate finding this solution, in some implementations, multiple simulations may be run in parallel (e.g., paralley processing) with values sampled from a grid (e.g., a 2D grid—one dimension for each parameter). In some implementations, to find the value of the parameter that satisfies the set of target criteria, the EFA process can perform a grid search first followed by, for example, a root finding method, to improve the convergence of the root finding method by dividing the domain into locally convex segments, or simply by reducing the number of sequential steps in the root finding method. In some implementations, the therapeutic purpose is to determine feasibility of developing the therapeutic drug. In some implementations, the EFA process 200 includes comparing the at least one value of the at least one parameter with a set of predetermined values of the at least one parameter (e.g., existing and plausible values of the therapeutic drug and biological parameters) to determine feasibility of developing the therapeutic drug

At step 210, the EFA process 200 includes sending the set of simulation results and at least one value of at least one parameter when at least one value exists. In some implementations, the EFA process 200 includes presenting the set of simulation results in an interactive graphical user interface that allows a user to adjust the set of parameters and the set of target criteria to analyze a correlation between the set of parameters and the set of target criteria. In some implementations, the set of simulation results is cached based on the definition of the simulations or a hash of the definition of the simulations to avoid having to simulate a result that was previously simulated. This can include single trajectories, or whole analysis workflows. For example, the EFA process 200 can include storing the user selected models and parameters in a cache memory that can be retrieved by the processor to quickly repeat the simulation and generate simulation results. In some implementations, the interactive graphical user interface can be called from a scripting environment or through an application programming interface.

In some implementations, the EFA process 200 includes generating, presenting (or visualizing) plots of the simulation results. The output plots can include, for example, criteria vs scan parameter (i.e., the value of the selected criterion for each value of the 1D scan parameter simulated), criteria heatmap (i.e., the value of the selected criterion for pair of 2D scan parameters), criterion time course (i.e., a time course plot of the selected criterion's time value), free drug (i.e., time course plot of the concentration of drug not bound to anything), total drug (i.e., a time course plot of the concentration of soluble drug) and/or the like.

### EXAMPLE

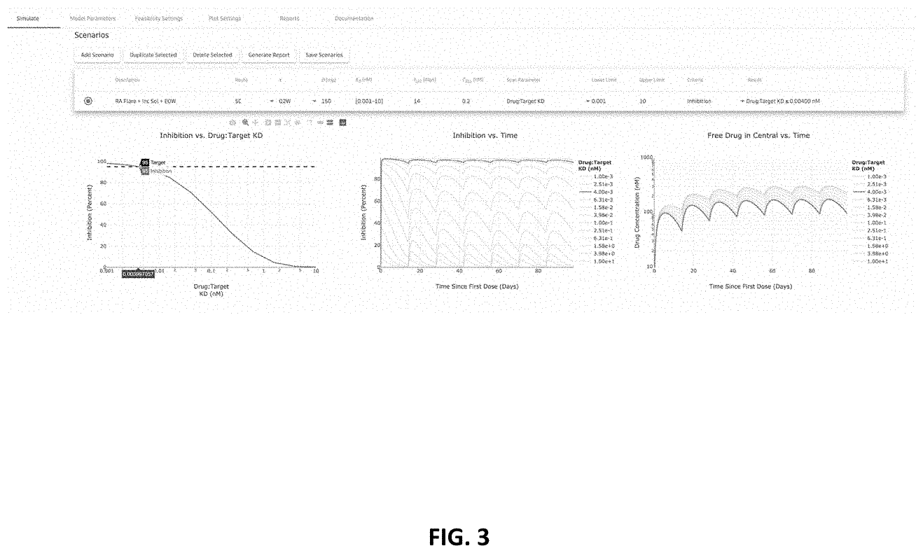

In an example, a user desires to explore the binding affinity of a therapeutic drug candidate for a specific therapeutic purpose. The user provides, via a user interface at the client device, input selecting a monospecific anti-ligand QSP model based on the interactions between the therapeutic drug candidate and the target. The user then provides, via the user interface at the client device, input defining (1) a value of a first subset of the set of parameters of the QSP model (e.g., every two-week subcutaneous dosing) and (2) a set of target criteria (e.g., 95% inhibition). The server can generate a set of simulation results (e.g., FIG. 3) based on the monospecific anti-ligand QSP model, the value of the first subset of the set of parameters (e.g., every two-week subcutaneous dosing) and (2) a set of target criteria (e.g., 95% inhibition) after a defined number of repeat subcutaneous doses. In some implementations, the user can move the cursor (e.g., a mouse coupled to the client device) in any of the plots on the user interface and the corresponding plots can change based on the position of the user's cursor. In FIG. 3, when the cursor moves to 95% inhibition, the server provides, via the user interface, the binding affinity of the therapeutic drug candidate as 0.003997057 nM (the critical value of a parameter that satisfies the target criteria). In some implementations, for example, the server may provide the feasible value of a therapeutic binding affinity to be <0.003 nM.

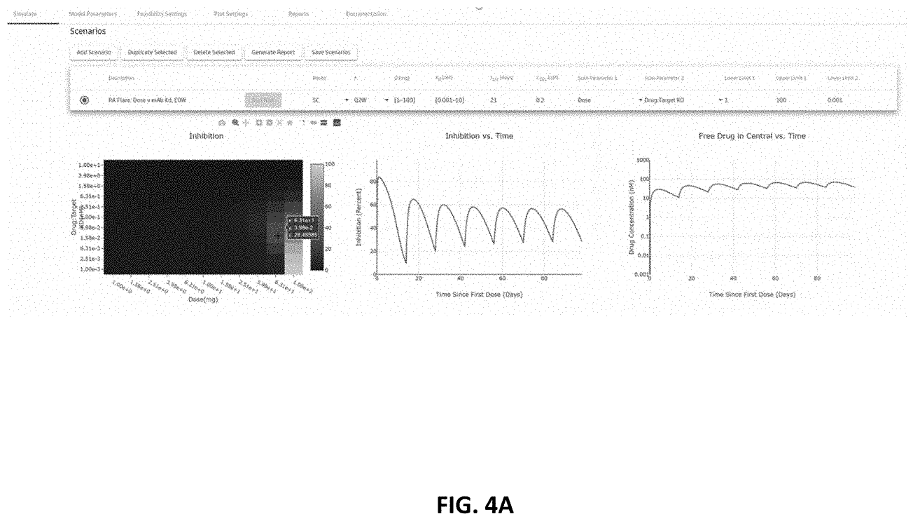

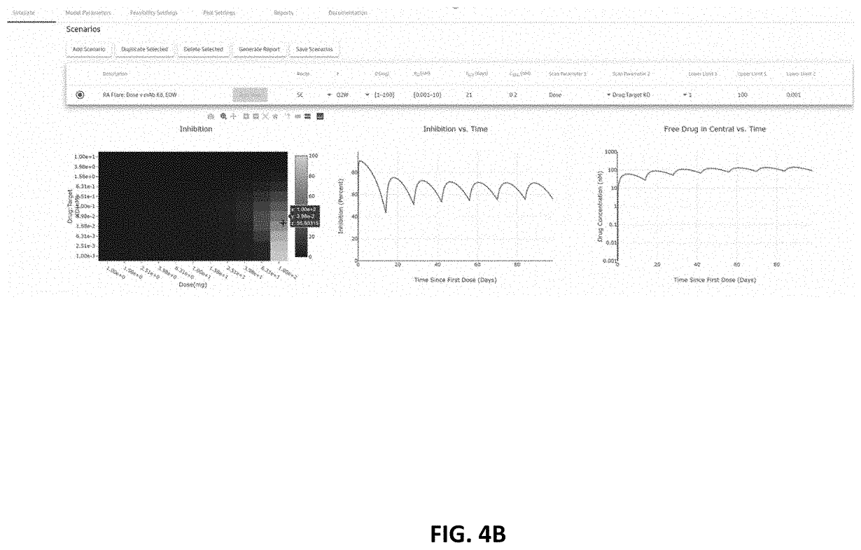

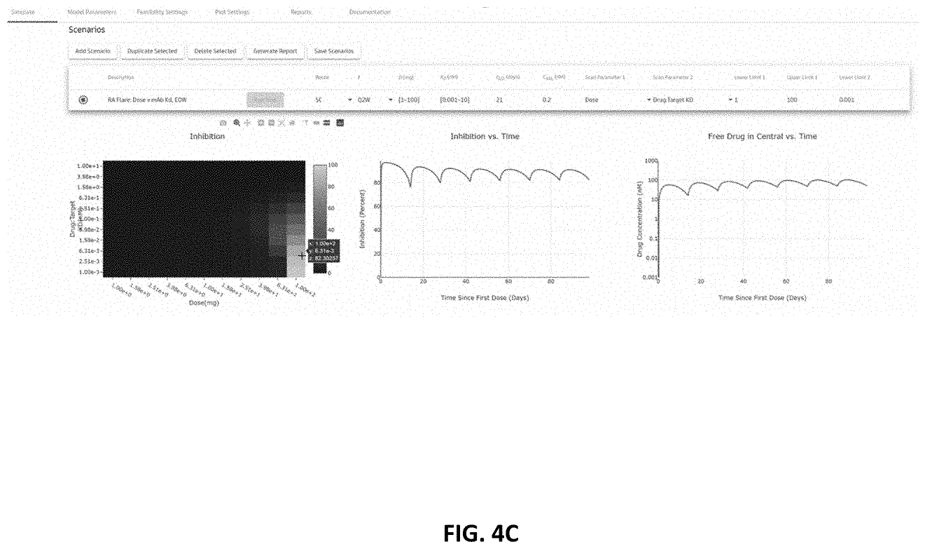

In some implementations, the server can receive user input from the client device to vary a value of the first subset of the set of parameters (e.g., dose, binding affinity) to determine the optimal and/or desired parameter ranges to meet the target criteria. As shown in FIGS. 4A-4C, the user can move the cursor on the user interface in the graph of dose, inhibition and binding affinity, the corresponding plots of, for example, time since first dose vs inhibition changes to correspond to where the cursor is in the first graph.

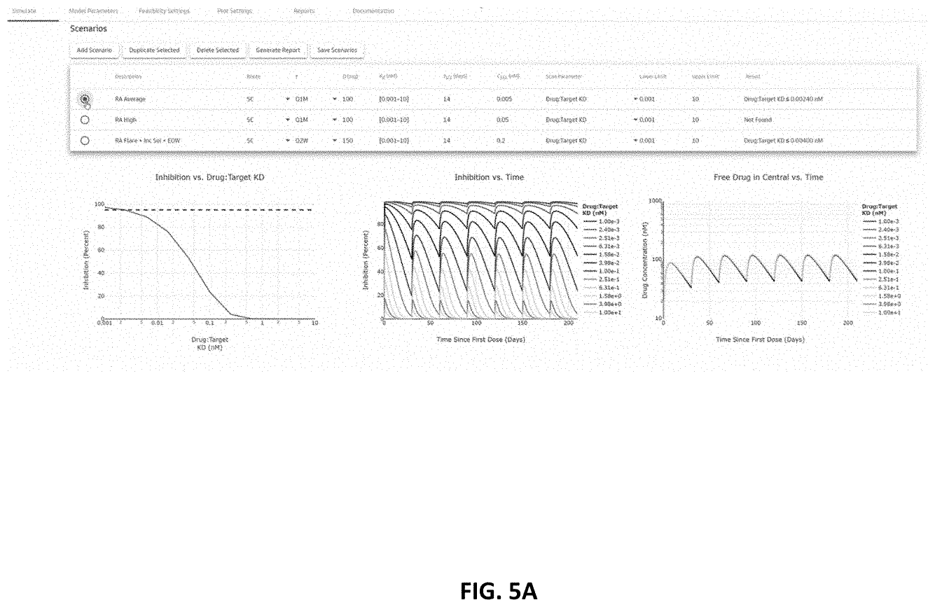

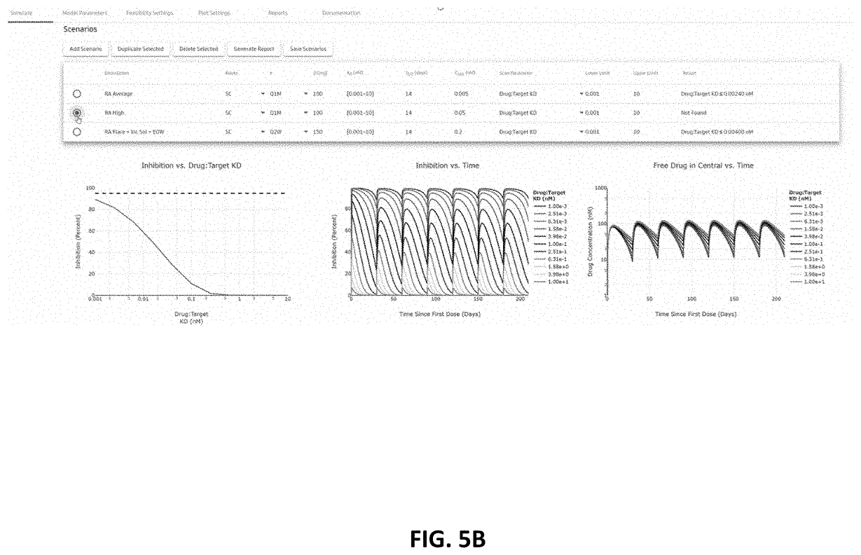

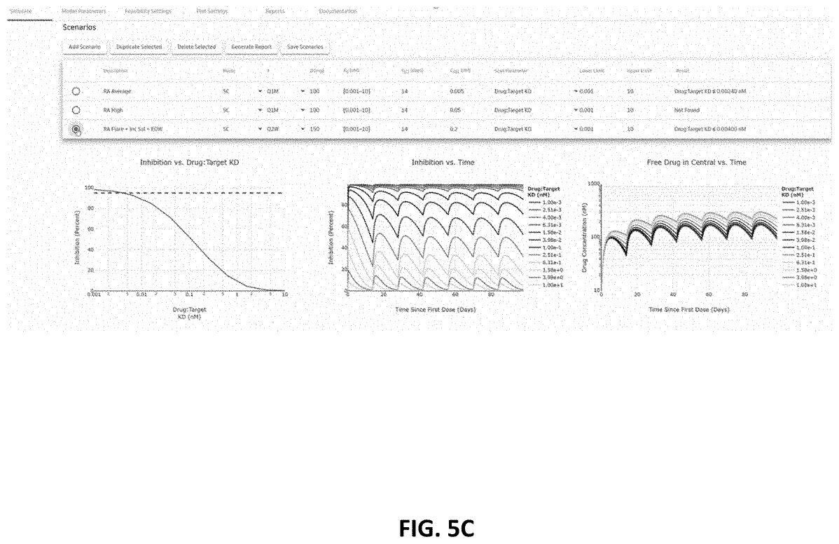

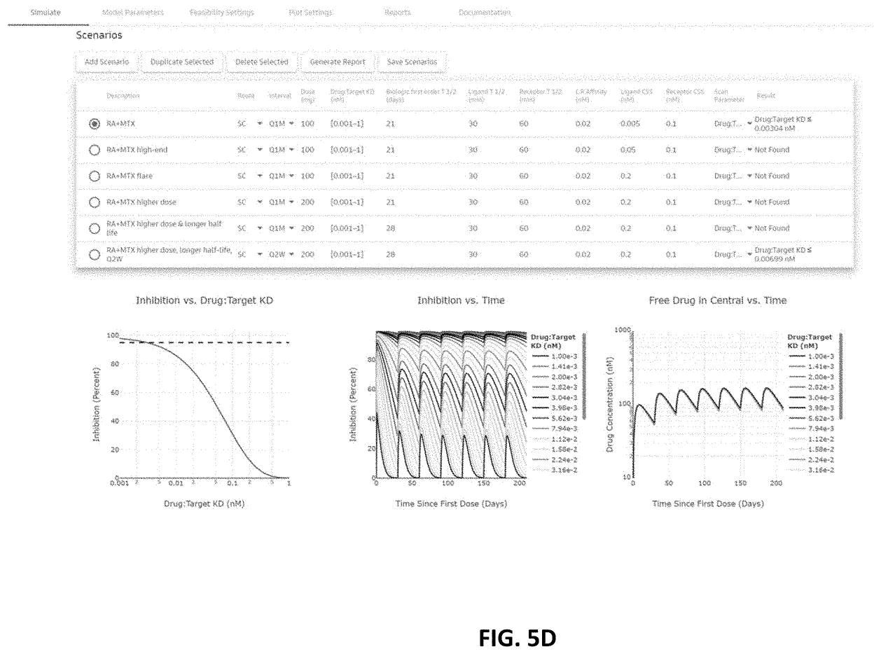

In some implementations, the EFA process allows the user to define different scenarios that the user desires to assess or compare and see how the PK/PD for each scenario varies and if the target criteria can be met. For example, as shown in FIGS. 5A-5C, the server can generate simulation results (e.g., inhibition vs binding affinity) and the feasibilities for different scenarios of various patient groups, as represented by different biological characteristics or parameters, such as average patients (FIG. 5A), patients with high levels of disease marker (FIG. 5B), and patients with extremely high levels of disease (FIG. 5C). In other words, the EFA process allows the user to quickly and interactively through the user interface assess, for example, whether a therapeutic drug candidate can meet the target criteria, and what is the critical value or critical range of a parameter of the therapeutic drug candidate for subsets of patient subgroups or for all patients.

As shown in FIG. 3, FIGS. 4A-4C, and FIGS. 5A-5D, in some implementations, the server provides, via the user interface, multiple tabs for simulation, parameter values, settings, reports, and documentation. The simulate tab can display the scenarios for this selected model as well as the plots (showing the simulation results). A scenario is one set of parameters for a selected QSP model that can be applied to generate simulation results. The user can, via the user interface provided by the server, add or duplicate scenarios to quickly assess “what if” situations for the therapeutic drug candidate. As shown in FIG. 5D, each row in the scenarios table is a unique scenario and when selected, the corresponding plots are shown below.

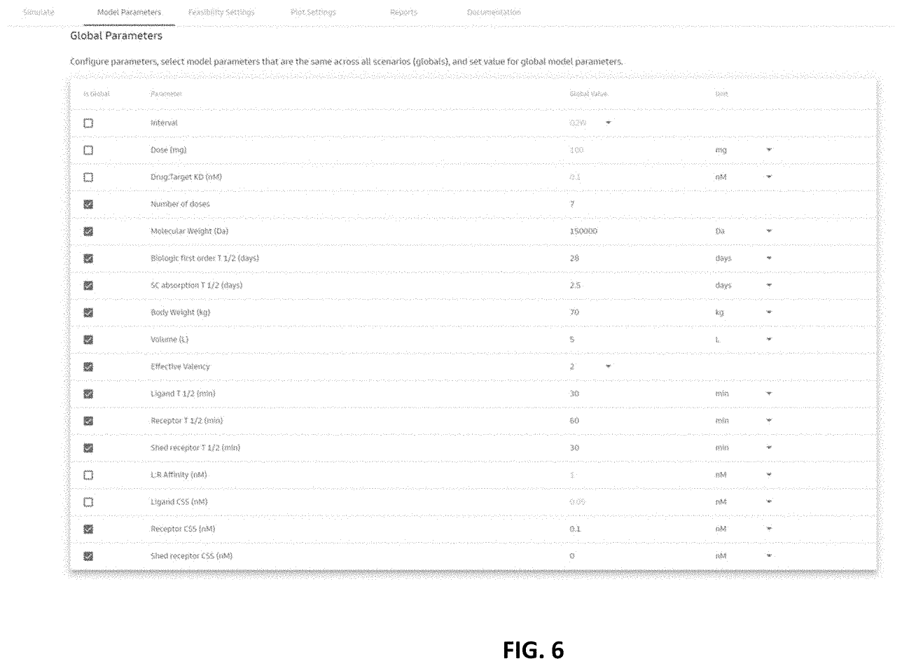

FIG. 6 shows the model parameters tab of the EFA user interface that lists the set of parameters included in the selected model and their default values. In some implementations, if a parameter in this list is checked, the parameter is global and the nominal value can remain constant throughout the scenarios and simulations (e.g., a set of nominal macro parameters which can include at least one of a time constant, a concentration, or a thermodynamic constant). When the parameter is not checked, the parameter is considered a local parameter and can appear as a column in the scenarios table in the simulate tab.

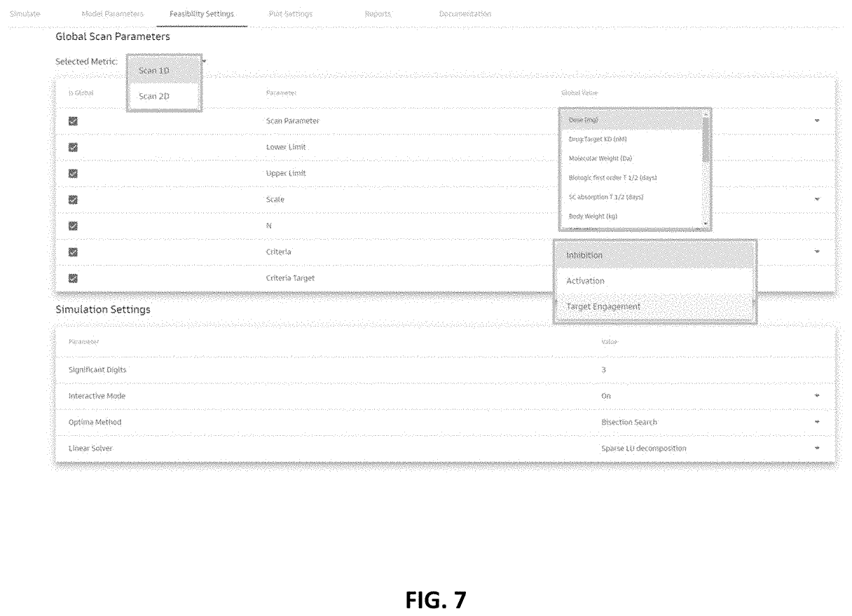

FIG. 7 shows the feasibility settings tab of the EFA user interface that includes the settings for the scan metrics and simulation settings. The server can provide 1D and 2D scans. Scan parameters can be any model parameter that the user desires to vary within user-defined limits. The server provides several target criteria such as percent inhibition, activation, target engagement, therapeutic index, concentrations or amounts of states in the model. The target criteria may also include or any formula of the above including min over time, max over time, value at a particular time, value at the end of the dose interval, average value or the integral over time of any specific time interval or over all time

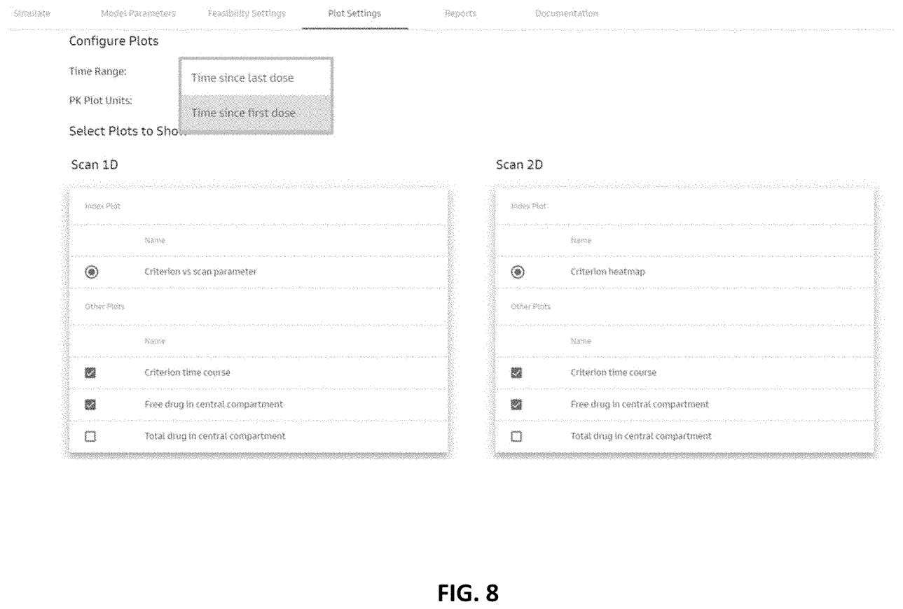

FIG. 8 shows the plot settings tab of the EFA user interface. In this plot settings tab, the server enables the user to vary plot settings such as time range and units. Time range includes, for example, time since the first dose or time since last dose. The user can also choose, via the EFA user interface, which plots are displayed for both 1D and 2D scans. In some implementations, the server can store the selected model, scenario data, and simulation plots in a report with the click of a button. Each report is timestamped and uniquely identified.

### Example

In this example, the user desires to assess a therapeutic drug candidate that meets the target RTP/TMP criteria as early on as possible. The server (e.g., server 101 as discussed in FIG. 1) provides to the client device a library of single- and multi-compartment models that includes a variety of pharmacologies and includes more detailed description of the models and parameters. The user desires to determine an acceptable binding affinity and first order half life given prerequisites such as dosing route and frequency for an anti-TNF monoclonal antibody under consideration for patients with rheumatoid arthritis who are on methotrexate. The client device sends a user input selecting the single compartment, anti-ligand QSP model to the server. In this example, it is assumed that the diseased joint is well perfused. The client device sends a second user input setting the target criteria as the target inhibition of greater than 95% ligand:receptor inhibition for the whole dosing interval.

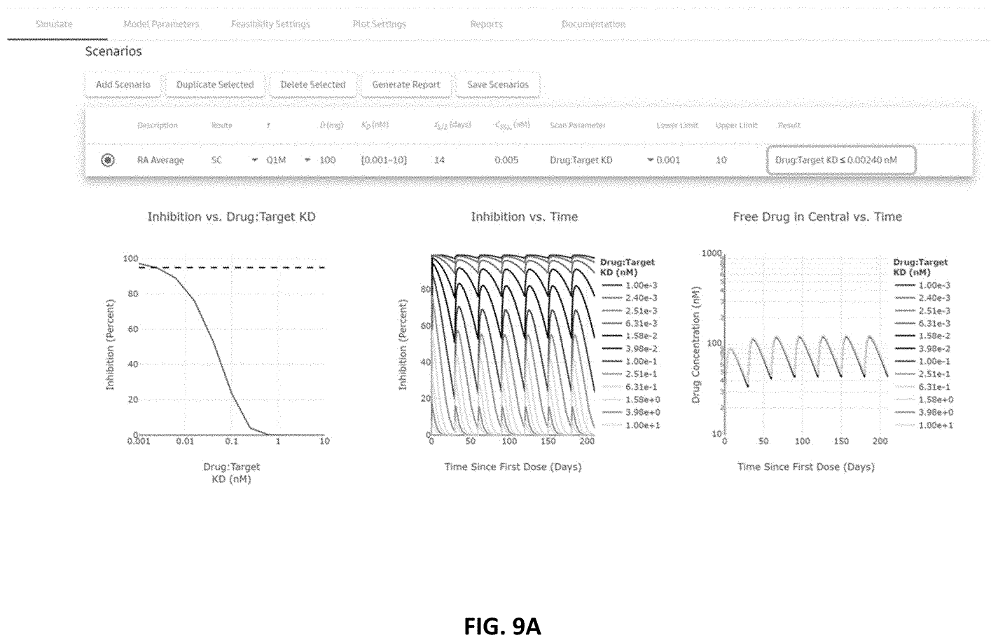

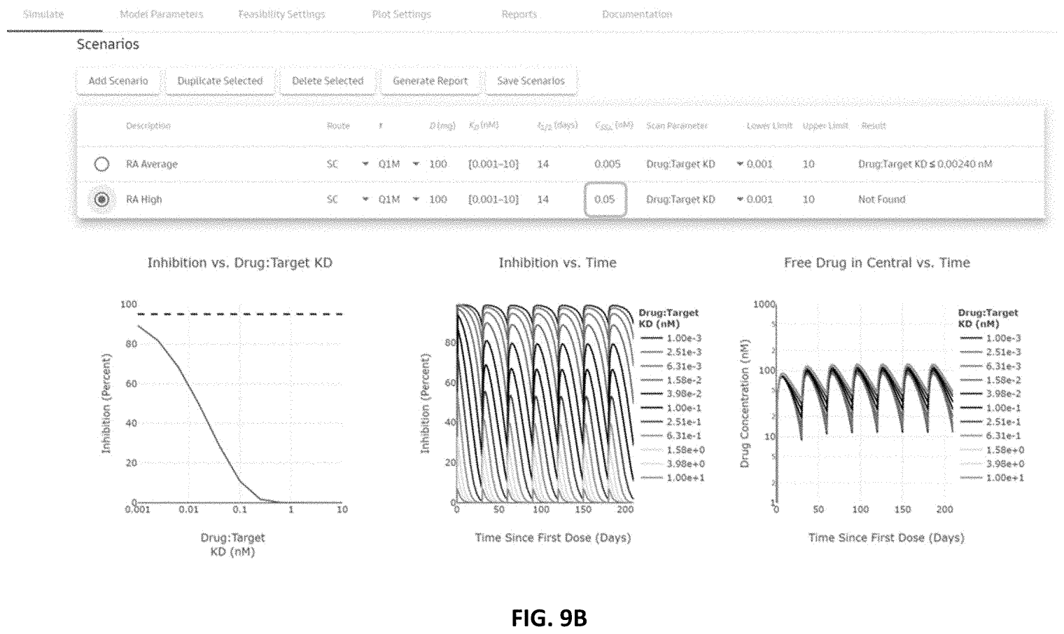

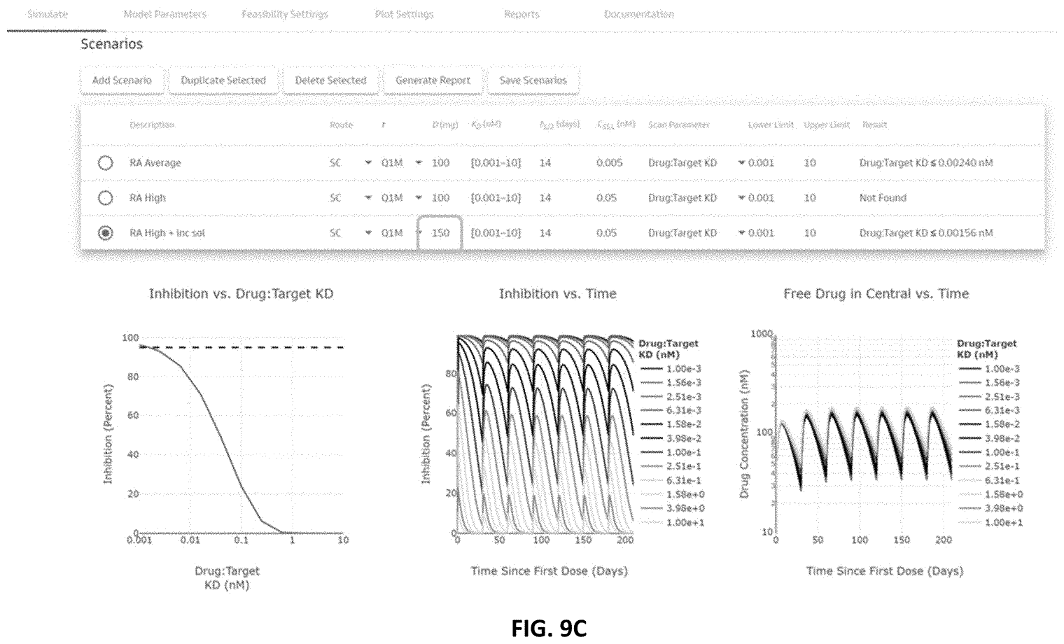

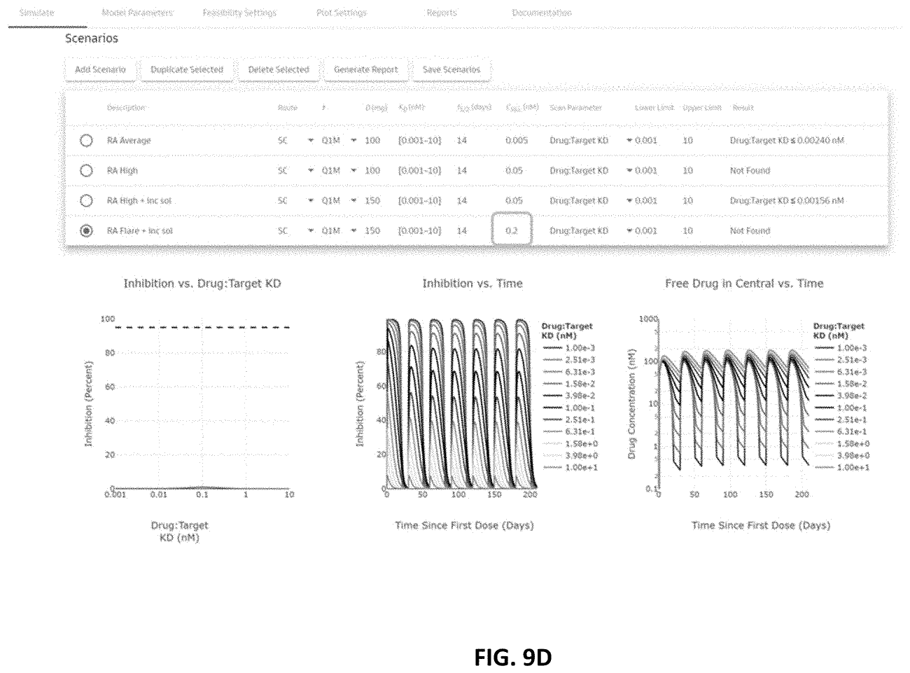

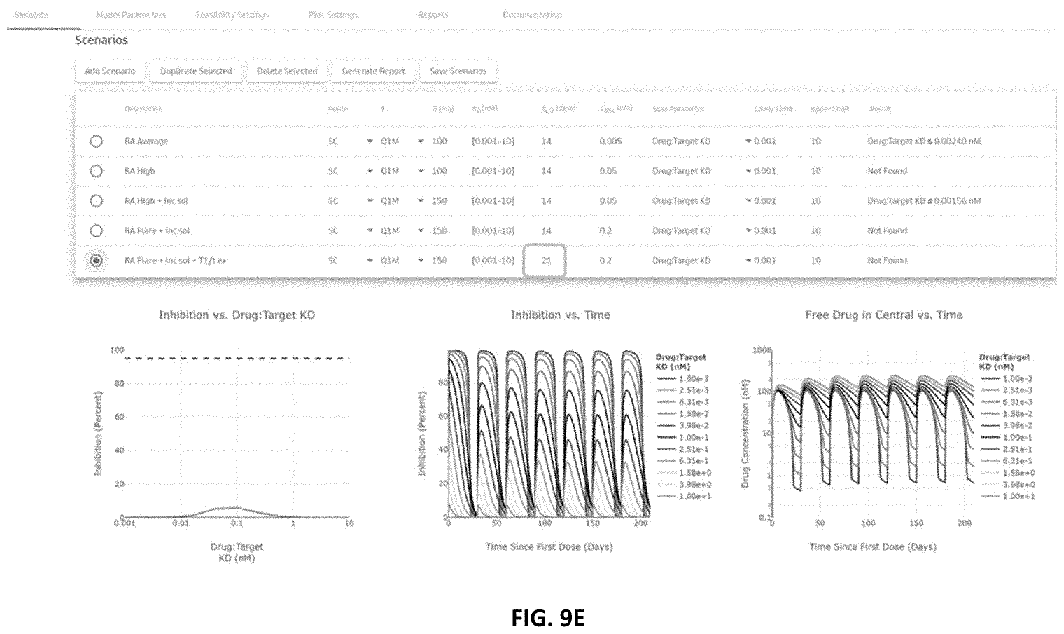

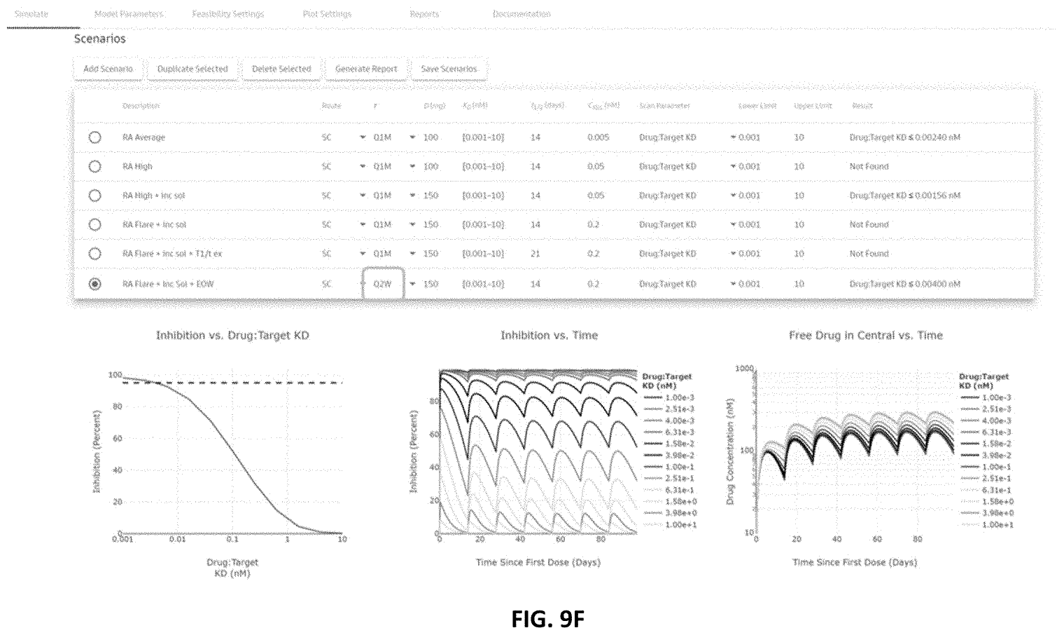

The server can receive user input from the client device building a scenario as shown in FIGS. 9A-9F. In some implementations, the server provides the EFA application and the computational capabilities to the client device. In some implementations, the server can provide the EFA application to the client device, and computing devices communicatively coupled to the server are configured to provide computation capabilities and send the simulation results to the server. As shown in FIG. 9A, if the server receives a user input of the parameter values for a nominal patient, the server (or the computing devices) generates the simulation results of 2.4 pm binding affinity. However, as shown in FIG. 9B, if the server receives a user input to increase the ligand concentration 10 fold to simulate a high-end scenario, the server (or the computing devices) generates the simulation results in which feasibility decreases compared to the target criteria. As shown in FIG. 9C, if the server receives a user input to increase solubility to 150 mgs, the server (or the computing devices) generates the simulation results in which the feasibility increases compared to the target criteria. In a flare situation, as shown in FIG. 9D, the server (or the computing devices) generates the simulation results of no feasibility. In some implementations, flares have a ligand concentration around 0.2 nm. As shown in FIG. 9E, in addition to increasing solubility, the server receives a user input to extend the biological first order half life to 21 days. The server (or the computing devices) generates the simulation results that the inhibition increases but not achieving the target >95% inhibition. As shown in FIG. 9F, the server receives a user input to change the dosing to every other week, the server (or the computing devices) generates simulation results that the target >95% inhibition with a 4 pm binder is achieved.

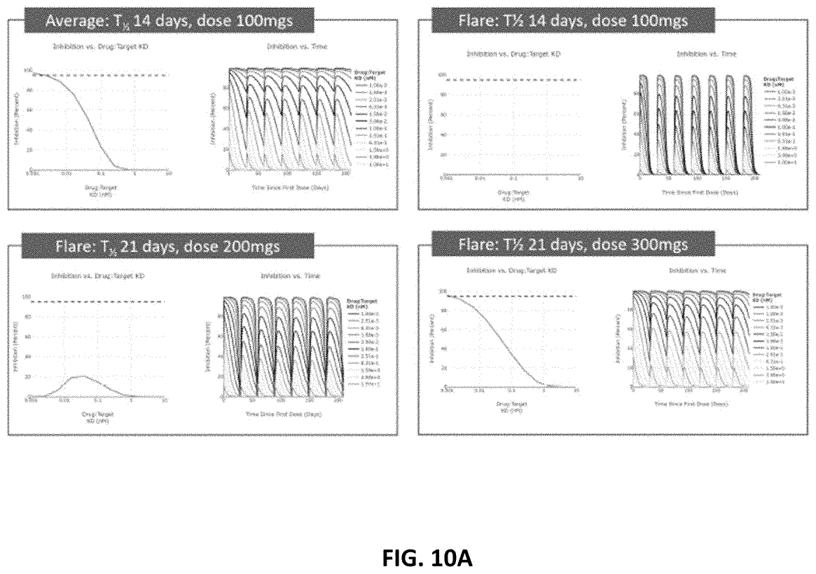

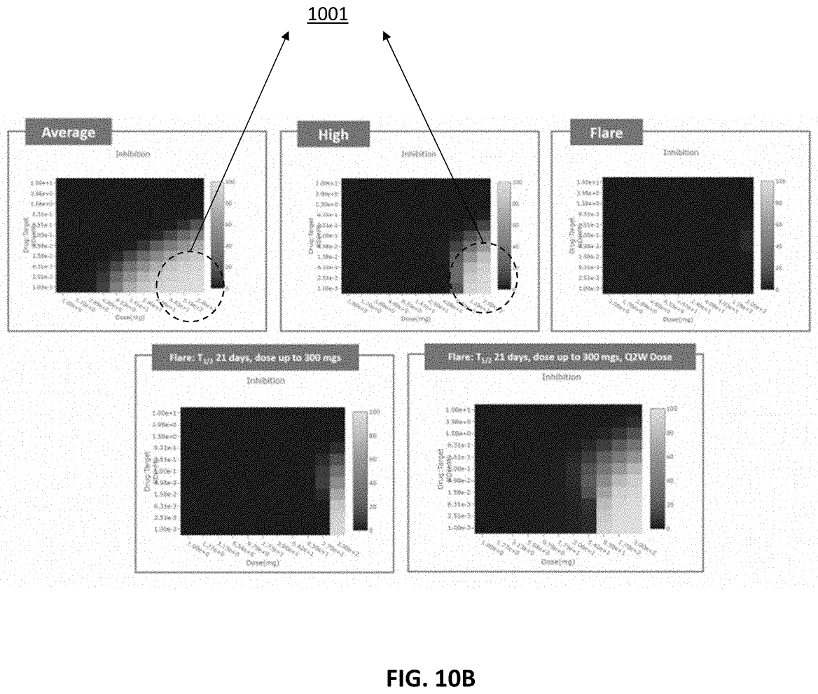

As shown in this example, the server (or the computing devices) provides both 1-D (as shown in FIGS. 9A-9F and FIG. 10A) and 2-D parameter scans (as shown in FIG. 10B). In some implementations, the 1-D scans are a useful tool if either one parameter range is the desired output of the analysis or if varying one parameter at a time is used to determine to which parameters the target biological system is most sensitive. In this example, the server (or the computing devices) generates a 1-D scan over Drug:Target binding Kd to determine if there is an optimal binding affinity that will work for all patient cases. As shown in FIG. 10A, for an average case (top left), the server (or the computing devices) generates the simulation results of a Kd of 2.4 pM achieve the success criteria of 95% inhibition. However, with the increased ligand concentration typically seen in a flare (top right), the server (or the computing devices) generates the simulation results that there is no Kd that will work.

The server provides the EFA capabilities to vary parameters to see which parameters have an impact on the developability of the therapeutic drug candidate. For example, extending biological half life and varying dose both show positive impacts on achieving the 95% inhibition criteria. Specifically, extending the half life to 21 days and varying dose up to 300 mgs both help this therapeutic achieve the 95% inhibition in the case of a flare.

In this example, the server (or the computing devices) can generate two-dimensional scans to help to find the optimal and/or desired combination of two model parameters to achieve a success criteria. In this example, the 1-D scan demonstrated that binding affinity (Kd) as well as dose have an impact on achieving the 95% inhibition criteria. Therefore, the server (or the computing devices) can perform the 2-D scanning over both binding affinity and dose to determine what combination of those parameters will work best for the scenarios. As shown in the top row of FIG. 10B, for the scanning dose from 1-200 mgs and Kd from 0.001 to 10 nM, the server (or the computing devices) generates the simulation results in some combinations that achieve 95% inhibition, as shown in the yellow region (1001), for average and high expression scenarios, but not in the case of the flare. Extending the half life to 21 days and varying the dose and dosing schedule both result in combinations of Kd and dose that achieve the 95% inhibition. Therefore, the server executing the EFA process provides the ideal Kd and dose amounts and frequency so that the next stage of drug development for this therapeutic drug candidate can be designed accordingly.

### Example

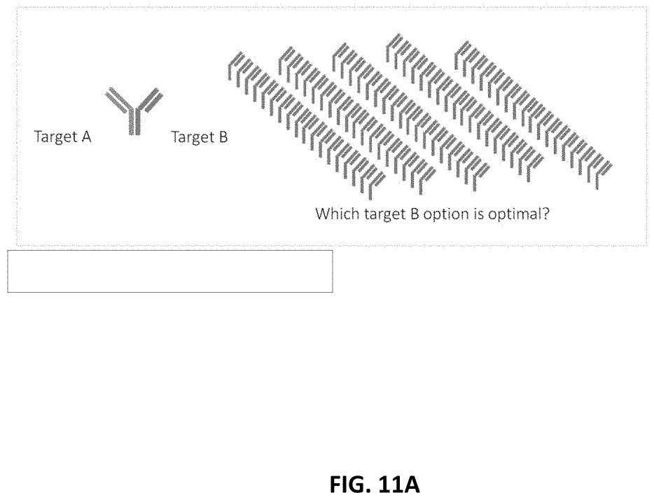

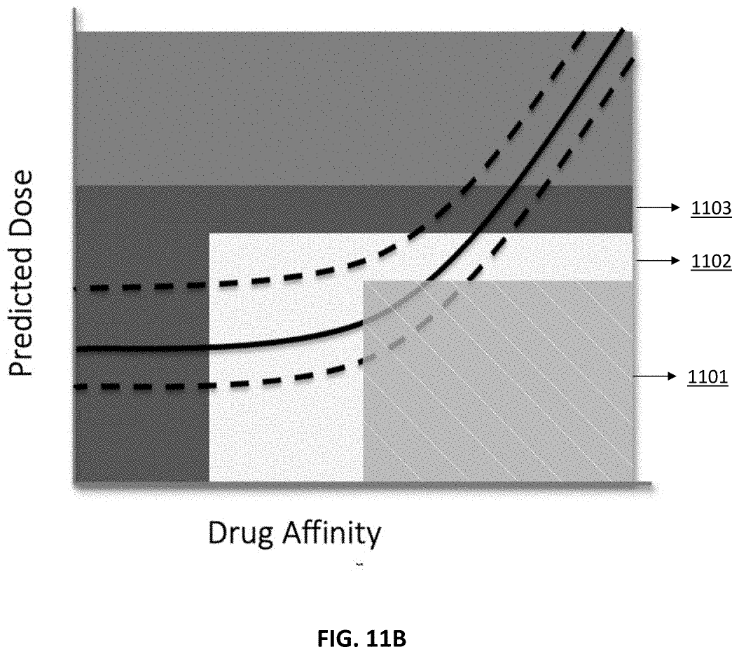

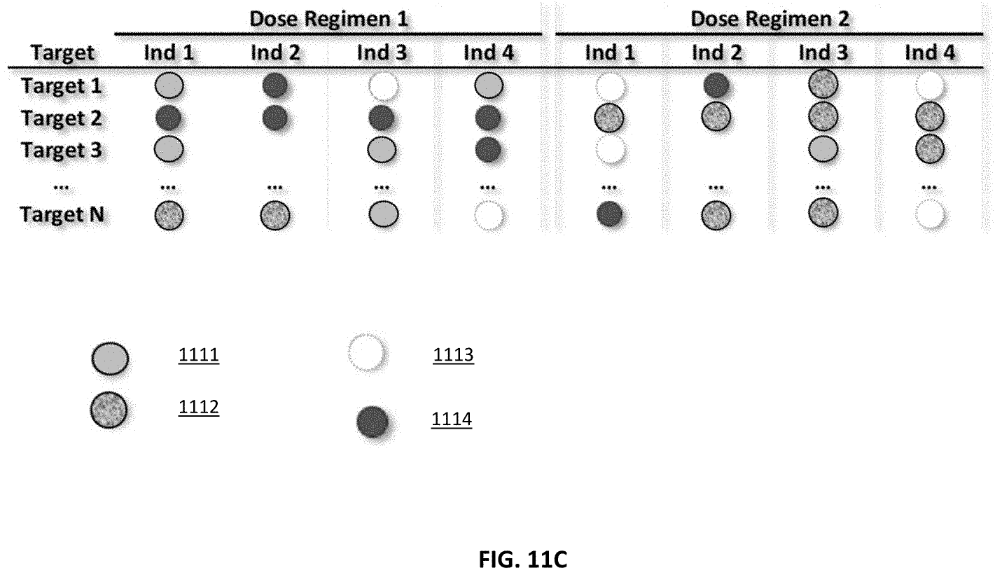

When designing a biological therapeutic agent (or a therapeutic drug candidate), the EFA process (e.g., the EFA process as discussed in FIG. 2) includes establishing the feasibility of achieving a desired target product profile (TPP) (or the target criteria) as early in the program as possible, typically at the ‘New Target Proposal Stage’ or at the start of Lead Identification (LI). In this example, the user desires to assess the feasibility of the bispecific agent at the target selection stage for four potential indications. One target, the anchor epitope (Target A), has been confirmed, but were undecided as to the second target (Target B); based on the biology of their indications, the user had 90 potential targets from which to choose (FIG. 11A). To be competitive, according to the TPP, the ideal dosing regimen was monthly, or less frequent, subcutaneous (SC) administration, and acceptable was weekly SC dosing. Due to resource constraints, the user can reduce the number of potential combinations to a low number, using the EFA process to prioritize a small set of target combinations to pursue further.

In response to the user's selection of the QSP model (e.g., Target A+B combination), the server (or the computing devices) generates simulation results including affinity vs dose requirement curves for each potential Target B (FIG. 11B). FIG. 11B shows an example dose vs. affinity curve for a hypothetical target. Developability regions are colored in green 1101, yellow 1102, and red 1103, corresponding with easy, medium, and difficult levels of developability respectively. The dotted lines indicate the window of uncertainty in the QSP simulations, based on factors such as antibody half-life, etc. Preferred targets are those where their curves go through the yellow 1102 and green 1101 regions, indicating that they would be relatively easy to develop.

The graph in FIG. 11B illustrates the relationship between affinity and dose. In some implementations, as the affinity of an antibody for its target increases, the dose for maintaining at least 90 percent target inhibition for the whole dosing interval decreases; however, a tradeoff exists between affinity and developability, i.e. it is harder to develop a high affinity inhibitor. Solubility is also another concern in developing a biologic, that is, it may be relatively easy to develop a bispecific at 100 mg/ml, but more difficult at 150 mg/ml. Therefore, the “sweet spot” is to develop an agent that is just potent enough to avoid high dosing, or high solubility; in FIG. 11B this “sweet spot” is colored green 1101.

Based on these curves, the server (or the computing devices) selects targets based on the level of difficulty in developing an agent that would meet the dosing requirement. For example, targets where a very high-affinity antibody would be required to meet the dosing requirement are less attractive than those where a lower affinity antibody could meet the dosing requirement.

The EFA process reduces the search space from 90 to four (4) viable potential target B options. This results in developing a platform and projects that could be executed in a timely fashion and reasonable costs. This can be generalized to, e.g., 2 dosing regimens, 4 indication, and N targets (FIG. 11C). FIG. 11C shows an example: Prioritized target candidates for two dosing regimens across four indications. For example, colored/pattered circles indicate the ease of developing an antibody against the target that satisfies the dosing requirement for the indication (green=easy 1111, yellow=medium 1113, red=hard 1112, black=very hard/not possible 1114, blank=insufficient data). By significantly reducing the number of targets to explore, to the server eliminates dead ends before significant investment by the user, saving potentially millions of dollars and years of time, and accelerate timelines for promising drug candidates.

### Methods

The computation methods and processes discussed in this section can be performed by the server (e.g., the server 101 in FIG. 1) or the computing devices communicatively coupled to the server providing the computational capabilities, and used to generate the set of simulation results as described in steps 202-210 of the EFA process 200 described with regards to FIG. 2. The instructions of the computation method and process discussed herein are stored in a memory and executed by a processor. In some embodiments, the server (or the computing devices) can be configured to store the drug pharmacology, target biology, and physiology as a system of biochemical equations in a memory. These equations may be mass action equations in which case rate laws can be automatically derived, or have explicit rate laws provided. In both cases the model is represented in a machine understandable format that allows for introspection of the system (the system of biochemical equations) and the automatic generation of data structures that represent mathematical terms need to solve and analyze the system.

These terms can be further compiled into a programming language, intermediate representation of a programming language, or machine code (such as C, C++, Fortran, Python, Java, Go, LLVM, Julia, x86, RISC) and then compiled into object code. Each step can be implemented in a model independent way which allows for optimization of numerical steps that will impact all potential models deployed to the system. This includes sequencing of calculations, elimination of redundant mathematical operations, optimization of generated code to allow for efficient compilation, or manipulating code to take advantage of hardware acceleration. The internal mathematical representations of models can be ordinary differential equations (ODE) supporting calculation of general derivatives such as the Jacobian matrix of the right hand side of the ODE system, the Hessian matrix, the sensitivity equations, or adjoint of the system. Use of these general derivatives support using, for example, large families of fast stiff solvers to speed up computation time. In addition, the software system leverages specialized methods to solve nonlinear and linear systems of equations required for numerical solution. For iterative solvers, preconditioners can be automatically derived to enable acceleration of model simulation.

The server (or the computing devices) is configured to define a set of reactions that describe the pharmacology of a drug and its associated biology. The server (or the computing devices) is configured to provide, via, for example, a user interface at the client device, detailed sets of models that are fit for purpose for the analysis of biotherapeutic drugs in the preclinical stage of development. The models contain sufficient mechanisms to enable answering key questions in the target identification, lead identification, and lead optimization process without being so complex as to be impractical without large data sets that are not available in early preclinical development.

The models may characterize ordinary chemical or biochemical reactions such as binding, unbinding, multimerization, enzymatic reactions, as well as multistep reactions such as protein synthesis, protein degradation, protein cleavage, mRNA or DNA synthesis, or degradation, cleavage or modification, post translational modifications such as phosphorylation, ubiquitination, glycosylation, methylation, acetylation and the like. The models may also characterize transport processes including intracellular transport reactions, transport between physiological compartments, or transport between cellular compartments such as early or late endosome, or transport between logical compartments such as discretized finite volumes, or areas. The models may also characterize cellular processes such as cell growth, cell doubling, apoptosis, phenotypic changes, cell differentiation, cell attachment, cell activation, cell inactivation, cell death and the like. The models may also characterize empirical pharmacokinetic descriptions of drug biology. The models may also characterize routes of administration which may be instantaneous or take place over time with a rate that can be an explicit function of time, or an implicit function defined in terms of a rate, a rule, or a system of algebraic equations or differential equations.

The server (or the computing devices) is configured to define the reactions using mass action chemical equations parameterized by 0th, 1st, and 2nd order rate constants (micro parameters), or aggregated rate laws which contain a mix of mass action reactions and thermodynamic equilibrium and or pseudosteady state, or other approximations, or a mix of ordinary differential equations and algebraic equations which may represent mass conservation rules, elimination of fast modes through the separation of timescales, or other more general model order reduction techniques such as singular perturbation analysis.

To enable linkage of the microprarameters to macroparameters (e.g., quantities that are experimentally accessible, quantities that are more familiar to scientists, or quantities that separate independent effects (such as kinetics from equilibrium behavior)), the server (or the computing devices) is configured to define algebraic relationships between macroparameters, and microparameters. These relationships between macroparameters, and microparameters may be combinations of repeated assignment rules, initial assignment rules, solutions to algebraic systems, differential-algebraic equations, or boundary conditions.

The server (or the computing devices) is configured to express these relationships in terms of complex systems of differential-algebraic equations that can be reduced to a solved form to enable efficient simulation. In some instances, the server (or the computing devices) is configured to perform a topological sort of an algebraic system, and perform numerical solutions of linear or nonlinear systems, perform partial solves and substitutions of algebraic systems, or by modifying differential equations to account for boundary conditions. The server (or the computing devices) is configured to generate the result as a system of rules and on the system of relationships that can be executed as fast procedural code augmenting a, potentially modified, system of differential-algebraic equations. In some instances, the server (or the computing devices) is configured to use “online” computation by using a general computer algebra system (CAS) framework to solve the equations, or a specialized CAS for particular subproblems. In some instances, the server (or the computing devices) is configured to provide the relationships and rules in a solved order generated by offline computation equations explicitly in solver order. The server (or the computing devices) is configured to define the relationships between the macroparameters and the microparameters by procedural code defined in general purpose programming language, domain specific languages, intermediate representations, compiled object code, or as pseudo code.

In some implementations, the server (or the computing devices) is configured to define relationships between the macro parameters and the micro parameters using total- or partial differentiation of the rules, rate laws and representations of the dynamic system, which can be further differentiated by the parameters and/or states to provide derivatives that can be used in sensitivity analysis, and in computing gradients for other numerical methods such as optimization. The server (or the computing devices) is configured to perform this computation through, for example, automatic differentiation, CAS based symbolic differentiation, finite differencing, complex step differencing, adjoint methods or other numerical methods. Where the server can provide the relationships as explicit code, the server can define the relationships via automatic code differentiation, or the derivatives provided explicitly, or the derivatives computed numerically by finite differencing, the complex finite step method, or other numerical methods. In some implementations, the server can use these defined relationships to calculate or approximate the critical values of parameters by, for example, a gradient-based method (e.g., Newtons methods).

In some implementations, the server (or the computing devices) is configured to generate simulation of the system by solving the system of ordinary differential equations or partial differential equations. The server (or the computing devices) is configured to generate (or receive) an initial, or boundary conditions of the system of equations. In some implementations, the server (or the computing devices) is configured to solve the initial condition algebraically from the macroparameters and microparameters as part of the relationships. The server (or the computing devices) can solve this system by precomputing once, and provides a significant speed increase for repeated simulations at different parameterizations. Alternatively, the server (or the computing devices) can solve the initial condition by, for example, numerically solving the same system of equations, by solving the right hand side of the differential equations (e.g., ODEs), or by running a sufficiently long simulation from an approximate initial condition.

In some implementations, the internal mathematical representations of models can be ordinary differential equations (ODE) supporting calculation of general derivatives such as the Jacobian matrix of the right hand side of the ODE system, the Hessian matrix, the sensitivity equations, or adjoint of the system. In some implementations, the server (or the computing devices) can record the simulation of the system with the right hand side (RHS) of the equations as a set of ordinary differential equations. To efficiently convert the set of chemical equations into differential equations, in some implementations, the server (or the computing devices) can translate the rate laws for the equations into a tensor algebra formulation of the RHS of the system of differential equations that represent the chemical kinetic system. In this formulation, the server (or the computing devices) can analytically differentiate the tensors to compute the Jacobian of the RHS (the matrix of derivatives of the right and side of the odes), the product of the Jacobian and an arbitrary vector, the Hessian of the RHS, the adjoint of the RHS, or other derivatives useful in numerical analysis. In addition to solving for the RHS, the server (or the computing devices) can optimize the tensor algebraic equations to reduce redundant calculations, maximize performance of computer memory access, take advantage of hardware acceleration, leverage GPUs or FPGAs for computation, and improve numerical stability of tensor calculations. The server (or the computing devices) can solve the RHS and/or optimize the tensor algebraic equations independent of model deployment, providing significant performance increase across models. For some models, the Jacobian and other tensor derivatives are sparse, so the server (or the computing devices) can provide sparse implementations of tensor algebra. Likewise, there are many duplicate terms in the matrix so the server (or the computing devices) can reorder the calculations to reduce the number of duplicate valuations to further increase the efficiency of solving the system. The combined tensor system for the ODEs, Jacobian, other tensor derivatives, and algebraic and differential equations are translated into computer code (for example C, C++, Go, Julia, Python, LLVM, bytecode, or assembly) and compiled on the fly to enable fast execution. To prevent redundant compilation, compiled objects are cached to eliminate duplicate compilation.

The server (or the computing devices) can implement one class of models using simulation by “stiff” solvers. For this class of models, the system leverages stiff ODE algorithms. For examples, such stiff methods include algorithms such as Rosenbrock, Implicit Runge-Kutta, Implicit-Explicit Methods, Backward Differentiation Formulas (BDF), Numerical Differentiation Formulas (NDF), Exponential Integrators (including exponential variants of the previously enumerated algorithms), and others.

Stiff solvers can depend on solving a nonlinear system of algebraic equations. The server (or the computing devices) can use Newton's method to solve the system which decomposes the nonlinear system into an iterated solution of linear systems. The server (or the computing devices) can solve the linear system using dense or sparse direct solvers such as dense or sparse LU decompositions, iterative numerical methods such as the generalized minimal residual method, fast approximation methods, exact solution methods, or other numerical methods. For some classes of problems and solvers, the system leverages specialized Krylov subspace solvers.

In some implementations, the server (or the computing devices) can use the partial or total derivatives of the solved system with respect to one or more input parameters. The server (or the computing devices) can use techniques of forward sensitivity analysis or adjoint sensitivity analysis. The stiff solvers are optimized and/or configured to reuse calculations for accelerated calculation of derivatives.

In some implementations, to solve for state vs time variable order, a stiff multi step solver is used to compute the value of the model states as a function of time. This can be solved using methods such as solvers based on numerical differentiation formulas or backward differentiation formulas. Likewise the solver may use multiple methods to solve the linear problem such as “Sparse LU Decomposition”, “Sparse GMRes”.

### Example QSP Models in the EFA System

Example QSP models that can be used in the EFA process (e.g., the EFA process 200 described with respect to FIG. 2) by the EFA system (e.g., the EFA system 100 described with respect to FIG. 1) can include (1) bispecific anti-ligand x anti-ligand, (2) bispecific anti-ligand x anti-ligand (4-compartment), (3) bispecific anti-ligand x anti-receptor, (4) bispecific anti-ligand x anti-receptor (4-compartment), (5) bispecific anti-ligand x anti-receptor with avidity (4-compartment), (6) monospecific anti-ligand, (7) monospecific anti-ligand (4-compartment), (8) monospecific anti-ligand (4-compartment), (9) bispecific anti-receptor x anti-receptor, (10) bispecific anti-receptor x anti-receptor (4-compartment), (11) bispecific anti-receptor x anti-receptor with avidity (4-compartment), (12) monospecific anti-receptor, (13) monospecific anti-receptor (4-compartment), (14) monospecific anti-receptor with avidity (4-compartment), (15) T-cell engager for solid tumors, (16) and/or other models. These example QSP models can be stored in the memory 112 at the server 101 or the memory 122 at the client 102, and executed by the processor 111 at the server 101 or the processor 121 at the client 102 as discussed with respect to FIG. 1.

In some implementations, the bispecific anti-ligand x anti-ligand example model can include an one-compartment model of bivalent antibody binding to two different soluble ligands. A bispecific biotherapeutic that binds to two different soluble ligand targets can prevent the ligands from binding to their cognate-receptors. The molecule can be mono- or bivalent for each target. This is a one-compartment model and there is an option for +/− soluble receptor. FIGS. 12A-12B show schematic diagrams illustrating a biotherapeutic scenario of the bispecific anti-ligand x anti-ligand example model, according to some embodiments. Example parameters of the bispecific anti-ligand x anti-ligand example model in the EFA system are shown in Table 1. Example target criteria (e.g., for the therapeutic drug candidate and the target for the therapeutic purpose) of the bispecific anti-ligand x anti-ligand example model in the EFA system are shown in Table 2. Example output plots of the bispecific anti-ligand x anti-ligand example model in the EFA system are shown in Table 3.

Table 1. Example parameters of the bispecific anti-ligand x anti-ligand example model in the EFA system.

| Symbol | Name | Default | Definition |
|---|---|---|---|
| τ | Interval | 14 | The interval between doses of the |
| | | | drug. |
| D | Dose (mg) | 100 | The amount of each dose. |
| K_{D, L1} | Drug: Target 1 KD (nM) | 0.1 | The affinity of the drug for ligand 1 |
| | | | as characterized by the equilibrium |
| | | | dissociation constant. This is the |
| | | | monovalent affinity (i.e. the affinity |
| | | | of single binding). |
| K_{D, L2} | Drug: Target 2 KD (nM) | 0.1 | The affinity of the drug for ligand 2 |
| | | | as characterized by the equilibrium |
| | | | dissociation constant. This is the |
| | | | monovalent affinity (i.e. the affinity |
| | | | of single binding). |
| N_{doses} | Number of doses | 7 | The number of doses to be given. |
| MW | Molecular Weight (Da) | 150000 | The molecular weight of the drug. |
| | | | This is used to convert the dose |
| | | | which is expressed in mass units to a |
| | | | molar dose. |
| t_{1/2} | Biologic first order T ½ (days) | 28 | The first-order elimination rate of the |
| | | | drug. |
| t_{1/2, a} | SC absorption T ½ (days) | 2.5 | The first-order absorption rate of the |
| | | | drug from the subcutaneous |
| | | | compartment into the central |
| | | | compartment. |
| BW | Body Weight (kg) | 70 | The body weight of the subject. This |
| | | | is used to convert the dose when |
| | | | expressed in units of mg/kg. |
| V | Volume (L) | 5 | The volume of the central |
| | | | compartment which is typically the |
| | | | plasma of the peripheral blood. |
| Valency₁ | Effective Valency 1 | 2 | The number of ligand 1 molecules |
| | | | that a molecule of drug can bind. |
| | | | This may be 1 or 2. |
| Valency₂ | Effective Valency 2 | 2 | The number of ligand 2 molecules |
| | | | that a molecule of drug can bind. |
| | | | This may be 1 or 2. |
| t_{1/2, L1} | Ligand 1 T ½ (hr) | 0.5 | The first-order elimination rate of |
| | | | ligand 1 from the central |
| | | | compartment. |
| t_{1/2, L2} | Ligand 2 T ½ (hr) | 0.5 | The first-order elimination rate of |
| | | | ligand 2 from the central |
| | | | compartment. |
| t_{1/2, R1} | Receptor 1 T ½ (hr) | 1 | The first-order turnover rate of |
| | | | receptor 1 in all compartments. |
| t_{1/2, R2} | Receptor 2 T ½ (hr) | 1 | The first-order turnover rate of |
| | | | receptor 2 in all compartments. |
| t_{1/2, sR1} | Soluble receptor 1 T ½ (hr) | 0.5 | The first-order elimination rate of |
| | | | soluble receptor 1 from the central |
| | | | compartment. |
| t_{1/2, sR2} | Soluble receptor 2 T ½ (hr) | 0.5 | The first-order elimination rate of |
| | | | soluble receptor 2 from the central |
| | | | compartment. |
| K_{D, L1:R1} | L1:R1 Affinity (nM) | 1 | The binding affinity of ligand 1 for |
| | | | receptor 1 as characterized by the |
| | | | equilibrium dissociation constant. |
| K_{D, L2:R2} | L2:R2 Affinity (nM) | 1 | The binding affinity of ligand 2 for |
| | | | receptor 2 as characterized by the |
| | | | equilibrium dissociation constant. |
| C_{SS, L1} | Ligand 1 CSS (nM) | 0.05 | The steady-state concentration of |
| | | | free ligand 1 in the compartment in |
| | | | the absence of drug. Ligand 1 bound |
| | | | to receptor 1 is not included in this |
| | | | quantity. |
| C_{SS, L2} | Ligand 2 CSS (nM) | 0.05 | The steady-state concentration of |
| | | | free ligand 2 in the compartment in |
| | | | the absence of drug. Ligand 2 bound |
| | | | to receptor 2 is not included in this |
| | | | quantity. |
| C_{SS, R1} | Receptor 1 CSS (#/cell) | 10000 | The steady-state concentration of |
| | | | receptor 1 in the compartment in the |
| | | | absence of drug. Receptor 1 bound to |
| | | | ligand 1 is included in this quantity, |
| | | | but soluble receptor 1 is not. |
| C_{SS, R2} | Receptor 2 CSS (#/cell) | 10000 | The steady-state concentration of |
| | | | receptor 2 in the compartment in the |
| | | | absence of drug. Receptor 2 bound to |
| | | | ligand 2 is included in this quantity, |
| | | | but soluble receptor 2 is not. |
| C_{SS, sR1} | Soluble receptor 1 CSS (nM) | 0 | The steady-state concentration of |
| | | | soluble receptor 1 in the |
| | | | compartment in the absence of drug. |
| C_{SS, sR2} | Soluble receptor 2 CSS (nM) | 0 | The steady-state concentration of |
| | | | soluble receptor 2 in the |
| | | | compartment in the absence of drug. |
| Density_{cells} | Cell density (#/mL) | 1000000 | Density of receptor-bearing cells in |
| | | | the compartment. |
| Scale_{KD, L1} | Drug: Target 1 Kd scale | 1 | A multiplicative factor for the |
| | | | equilibrium dissociation constant |
| | | | between drug and ligand 1 in the |
| | | | compartment. Can be used to model |
| | | | drugs whose target affinity is |
| | | | affected by the compartment. |
| Scale_{KD, L2} | Drug: Target 2 Kd scale | 1 | A multiplicative factor for the |
| | | | equilibrium dissociation constant |
| | | | between drug and ligand 2 in the |
| | | | compartment. Can be used to model |
| | | | drugs whose target affinity is |
| | | | affected by the compartment. |

TABLE 2
Example target criteria of the bispecific anti-ligand ×
anti-ligand example model in the EFA system.

| | Default | | |
|---|---|---|---|
| Criterion | Target | Units | Description |
| Inhibition | 90 | percent | Inhibition of ligand receptor complex. |
| | | | Measured as a percent of untreated. |
| Target | 90 | percent | Percent reduction of free target |
| Engagement | | | due to drug binding. Measured as |
| | | | a percent of untreated. |
| Activation | 90 | percent | Percent of receptor occupied by |
| | | | ligand or drug. Measured as a |
| | | | percent of total. This is |
| | | | typically used for agonists. |

TABLE 3
Example output plots of the bispecific anti-ligand ×
anti-ligand example model in the EFA system.

| Output Plot | Description |
|---|---|
| Criterion | The value of the selected criterion |
| vs scan | for each value of the 1D scan |
| parameter | parameter simulated. The desired target |
| | value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is any form of the |
| | drug not bound to any membrane proteins. |
| | This may include drug bound to one or |
| | more soluble proteins such as |
| | ligands or soluble receptors. |

The example bispecific anti-ligand x anti-ligand (4-compartment) model can include a four-compartment model of bivalent antibody binding to two different soluble ligands. The bispecific biotherapeutic that binds to two different soluble ligand targets and prevents the ligands from binding to their cognate-receptors. The molecule can be mono- or bivalent for each target. This is a four-compartment model with a central, peripheral, disease and tox compartments. There is an option for +/− soluble receptor for each receptor. FIG. 12A shows a schematic diagram illustrating a biotherapeutic scenario of the example bispecific anti-ligand x anti-ligand (4-compartment) model, according to some embodiments. Example parameters of the bispecific anti-ligand x anti-ligand (4-compartment) model in the EFA system are shown in Table 4. Example target criteria (e.g., for the therapeutic drug candidate and the target for the therapeutic purpose) of the bispecific anti-ligand x anti-ligand (4-compartment) example model in the EFA system are shown in Table 5. Example output plots of the bispecific anti-ligand x anti-ligand example model (4-compartment) in the EFA system are shown in Table 6.

TABLE 4
Example parameters of the bispecific anti-ligand ×
anti-ligand (4-compartment) model in the EFA system.

| Symbol | Name | Default | Definition |
|---|---|---|---|
| τ | Interval | 14 | The interval between doses of |
| | | | the drug. |
| D | Dose (mg) | 100 | The amount of each dose. |
| K_{D, L1} | Drug: Target | 0.1 | The affinity of the drug for |
| | 1 KD (nM) | | ligand 1 as characterized by |
| | | | the equilibrium dissociation |
| | | | constant. This is the |
| | | | monovalent affinity (i.e. the |
| | | | affinity of single binding). |
| K_{D, L2} | Drug: Target | 0.1 | The affinity of the drug for |
| | 2 KD (nM) | | ligand 2 as characterized by |
| | | | the equilibrium dissociation |
| | | | constant. This is the |
| | | | monovalent affinity (i.e. the |
| | | | affinity of single binding). |
| N_{doses} | Number of | 7 | The number of doses to be |
| | doses | | given. |
| MW | Molecular | 150000 | The molecular weight of the |
| | Weight (Da) | | drug. This is used to convert |
| | | | the dose which is expressed in |
| | | | mass units to a molar dose. |
| t_{1/2} | Biologic | 28 | The first-order elimination rate |
| | first | | of the drug. |
| | order | | |
| | T ½ (days) | | |
| t_{1/2, a} | SC absorption | 2.5 | The first-order absorption rate |
| | T ½ (days) | | of the drug from the |
| | | | subcutaneous compartment |
| | | | into the central compartment. |
| BW | Body | 70 | The body weight of the |
| | Weight (kg) | | subject. This is used to convert |
| | | | the dose when expressed in |
| | | | units of mg/kg. |
| V | Volume | 2.5 | The volume of the central |
| | central (L) | | compartment which is |
| | | | typically the plasma of the |
| | | | peripheral blood. |
| Vₚₑᵣᵢₚₕₑᵣₐₗ | Volume | 12.8 | The volume of the non-blood |
| | peripheral (L) | | fluids that the antibody can |
| | | | distribute to. |
| V_{disease} | Volume | 0.1 | The volume of the disease |
| | disease (L) | | compartment interstitial fluid. |
| Vₜₒₓ | Volume | 0.1 | The volume of the tox |
| | tox (L) | | compartment interstitial fluid. |
| T_{dist, peripheral} | Drug Tdist | 30 | The distribution rate of drug |
| | peripheral (hr) | | from the central compartment |
| | | | to the peripheral compartment. |
| T_{dist, disease} | Drug Tdist | 30 | The distribution rate of drug |
| | disease (hr) | | from the central compartment |
| | | | to the disease compartment. |
| T_{dist, tox} | Drug Tdist | 30 | The distribution rate of drug |
| | tox (hr) | | from the central compartment |
| | | | to the tox compartment. |
| P_{dist, peripheral} | Drug partition | 0.190625 | The steady-state ratio of |
| | coefficient | | soluble drug in the peripheral |
| | peripheral | | compartment to soluble drug in |
| | | | the central compartment. |
| P_{dist, disease} | Drug partition | 0.3 | The steady-state ratio of |
| | coefficient | | soluble drug in the disease |
| | disease | | compartment to soluble drug in |
| | | | the central compartment. |
| P_{dist, tox} | Drug partition | 0.3 | The steady-state ratio of |
| | coefficient | | soluble drug in the tox |
| | tox | | compartment to soluble drug in |
| | | | the central compartment. |
| Valency₁ | Effective | 2 | The number of ligand 1 |
| | Valency 1 | | molecules that a molecule of |
| | | | drug can bind. This may be 1 |
| | | | or 2. |
| Valency₂ | Effective | 2 | The number of ligand 2 |
| | Valency 2 | | molecules that a molecule of |
| | | | drug can bind. This may be 1 |
| | | | or 2. |
| t_{1/2, L1} | Ligand 1 | 0.5 | The first-order elimination rate |
| | T ½ (hr) | | of ligand 1 from the central |
| | | | compartment. |
| t_{1/2, L2} | Ligand 2 | 0.5 | The first-order elimination rate |
| | T ½ (hr) | | of ligand 2 from the central |
| | | | compartment. |
| t_{1/2, R1} | Receptor 1 | 1 | The first-order turnover rate of |
| | T ½ (hr) | | receptor 1 in all compartments. |
| t_{1/2, R2} | Receptor 2 | 1 | The first-order turnover rate of |
| | T ½ (hr) | | receptor 2 in all compartments. |
| t_{1/2, sR1} | Soluble | 0.5 | The first-order elimination rate |
| | receptor 1 | | of soluble receptor 1 from the |
| | T ½ (hr) | | central compartment. |
| t_{1/2, sR2} | Soluble | 0.5 | The first-order elimination rate |
| | receptor 2 | | of soluble receptor 2 from the |
| | T ½ (hr) | | central compartment. |
| K_{D, L1:R1} | L1:R1 | 1 | The binding affinity of ligand |
| | Affinity (nM) | | 1 for receptor 1 as |
| | | | characterized by the |
| | | | equilibrium dissociation |
| | | | constant. |
| K_{D, L2:R2} | L2:R2 | 1 | The binding affinity of ligand |
| | Affinity (nM) | | 2 for receptor 2 as |
| | | | characterized by the |
| | | | equilibrium dissociation |
| | | | constant. |
| C_{SS, L1, central} | Ligand 1 CSS | 0.05 | The steady-state concentration |
| | central (nM) | | of free ligand 1 in the central |
| | | | compartment in the absence of |
| | | | drug. Ligand 1 bound to |
| | | | receptor 1 is not included in |
| | | | this quantity. |
| C_{SS, L2, central} | Ligand 2 CSS | 0.05 | The steady-state concentration |
| | central (nM) | | of free ligand 2 in the central |
| | | | compartment in the absence of |
| | | | drug. Ligand 2 bound to |
| | | | receptor 2 is not included in |
| | | | this quantity. |
| C_{SS, L1, peripheral} | Ligand 1 CSS | 0.05 | The steady-state concentration |
| | peripheral (nM) | | of free ligand 1 in the |
| | | | peripheral compartment in the |
| | | | absence of drug. Ligand 1 |
| | | | bound to receptor 1 is not |
| | | | included in this quantity. |
| C_{SS, L2, peripheral} | Ligand 2 CSS | 0.05 | The steady-state concentration |
| | peripheral (nM) | | of free ligand 2 in the |
| | | | peripheral compartment in the |
| | | | absence of drug. Ligand 2 |
| | | | bound to receptor 2 is not |
| | | | included in this quantity. |
| C_{SS, L1, disease} | Ligand 1 CSS | 0.05 | The steady-state concentration |
| | disease (nM) | | of free ligand 1 in the disease |
| | | | compartment in the absence of |
| | | | drug. Ligand 1 bound to |
| | | | receptor 1 is not included in |
| | | | this quantity. |
| C_{SS, L2, disease} | Ligand 2 CSS | 0.05 | The steady-state concentration |
| | disease (nM) | | of free ligand 2 in the disease |
| | | | compartment in the absence of |
| | | | drug. Ligand 2 bound to |
| | | | receptor 2 is not included in |
| | | | this quantity. |
| C_{SS, L1, tox} | Ligand 1 CSS | 0.05 | The steady-state concentration |
| | tox (nM) | | of free ligand 1 in the tox |
| | | | compartment in the absence of |
| | | | drug. Ligand 1 bound to |
| | | | receptor 1 is not included in |
| | | | this quantity. |
| C_{SS, L2, tox} | Ligand 2 CSS | 0.05 | The steady-state concentration |
| | tox (nM) | | of free ligand 2 in the tox |
| | | | compartment in the absence of |
| | | | drug. Ligand 2 bound to |
| | | | receptor 2 is not included in |
| | | | this quantity. |
| C_{SS, R1, central} | Receptor 1 CSS | 10000 | The steady-state concentration |
| | central | | of receptor 1 in the central |
| | (#/cell) | | compartment in the absence of |
| | | | drug. Receptor 1 bound to |
| | | | ligand 1 is included in this |
| | | | quantity, but soluble receptor 1 |
| | | | is not. |
| C_{SS, R2, central} | Receptor 2 CSS | 10000 | The steady-state concentration |
| | central | | of receptor 2 in the central |
| | (#/cell) | | compartment in the absence of |
| | | | drug. Receptor 2 bound to |
| | | | ligand 2 is included in this |
| | | | quantity, but soluble receptor 2 |
| | | | is not. |
| C_{SS, R1, peripheral} | Receptor 1 CSS | 10000 | The steady-state concentration |
| | peripheral | | of receptor 1 in the peripheral |
| | (#/cell) | | compartment in the absence of |
| | | | drug. Receptor 1 bound to |
| | | | ligand 1 is included in this |
| | | | quantity, but soluble receptor 1 |
| | | | is not. |
| C_{SS, R2, peripheral} | Receptor 2 CSS | 10000 | The steady-state concentration |
| | peripheral | | of receptor 2 in the peripheral |
| | (#/cell) | | compartment in the absence of |
| | | | drug. Receptor 2 bound to |
| | | | ligand 2 is included in this |
| | | | quantity, but soluble receptor 2 |
| | | | is not. |
| C_{SS, R1, disease} | Receptor 1 CSS | 10000 | The steady-state concentration |
| | disease | | of receptor 1 in the disease |
| | (#/cell) | | compartment in the absence of |
| | | | drug. Receptor 1 bound to |
| | | | ligand 1 is included in this |
| | | | quantity, but soluble receptor 1 |
| | | | is not. |
| C_{SS, R2, disease} | Receptor 2 CSS | 10000 | The steady-state concentration |
| | disease | | of receptor 2 in the disease |
| | (#/cell) | | compartment in the absence of |
| | | | drug. Receptor 2 bound to |
| | | | ligand 2 is included in this |
| | | | quantity, but soluble receptor 2 |
| | | | is not. |
| C_{SS, R1, tox} | Receptor 1 CSS | 10000 | The steady-state concentration |
| | tox | | of receptor 1 in the tox |
| | (#/cell) | | compartment in the absence of |
| | | | drug. Receptor 1 bound to |
| | | | ligand 1 is included in this |
| | | | quantity, but soluble receptor 1 |
| | | | is not. |
| C_{SS, R2, tox} | Receptor 2 CSS | 10000 | The steady-state concentration |
| | tox | | of receptor 2 in the tox |
| | (#/cell) | | compartment in the absence of |
| | | | drug. Receptor 2 bound to |
| | | | ligand 2 is included in this |
| | | | quantity, but soluble receptor 2 |
| | | | is not. |
| C_{SS, sR1, central} | Soluble receptor | 0 | The steady-state concentration |
| | 1 CSS central | | of soluble receptor 1 in the |
| | (nM) | | central compartment in the |
| | | | absence of drug. |
| C_{SS, sR2, central} | Soluble receptor | 0 | The steady-state concentration |
| | 2 CSS central | | of soluble receptor 2 in the |
| | (nM) | | central compartment in the |
| | | | absence of drug. |
| C_{SS, sR1, peripheral} | Soluble receptor | 0 | The steady-state concentration |
| | 1 CSS peripheral | | of soluble receptor 1 in the |
| | (nM) | | peripheral compartment in the |
| | | | absence of drug. |
| C_{SS, sR2, peripheral} | Soluble receptor | 0 | The steady-state concentration |
| | 2 CSS peripheral | | of soluble receptor 2 in the |
| | (nM) | | peripheral compartment in the |
| | | | absence of drug. |
| C_{SS, sR1, disease} | Soluble receptor | 0 | The steady-state concentration |
| | 1 CSS disease | | of soluble receptor 1 in the |
| | (nM) | | disease compartment in the |
| | | | absence of drug. |
| C_{SS, sR2, disease} | Soluble receptor | 0 | The steady-state concentration |
| | 2 CSS disease | | of soluble receptor 2 in the |
| | (nM) | | disease compartment in the |
| | | | absence of drug. |
| C_{SS, sR1, tox} | Soluble receptor | 0 | The steady-state concentration |
| | 1 CSS tox | | of soluble receptor 1 in the tox |
| | (nM) | | compartment in the absence of |
| | | | drug. |
| C_{SS, sR2, tox} | Soluble receptor | 0 | The steady-state concentration |
| | 2 CSS tox | | of soluble receptor 2 in the tox |
| | (nM) | | compartment in the absence of |
| | | | drug. |
| T_{dist, L1, peripheral} | Ligand 1 Tdist | 30 | The distribution rate of ligand |
| | peripheral | | 1 from the central |
| | (hr) | | compartment to the peripheral |
| | | | compartment. |
| T_{dist, L2, peripheral} | Ligand 2 Tdist | 30 | The distribution rate of ligand |
| | peripheral | | 2 from the central |
| | (hr) | | compartment to the peripheral |
| | | | compartment. |
| T_{dist, L1, disease} | Ligand 1 Tdist | 30 | The distribution rate of ligand |
| | disease | | 1 from the central |
| | (hr) | | compartment to the disease |
| | | | compartment. |
| T_{dist, L2, disease} | Ligand 2 Tdist | 30 | The distribution rate of ligand |
| | disease | | 2 from the central |
| | (hr) | | compartment to the disease |
| | | | compartment. |
| T_{dist, L1, tox} | Ligand 1 Tdist | 30 | The distribution rate of ligand |
| | tox | | 1 from the central |
| | (hr) | | compartment to the tox |
| | | | compartment. |
| T_{dist, L2, tox} | Ligand 2 Tdist | 30 | The distribution rate of ligand |
| | tox | | 2 from the central |
| | (hr) | | compartment to the tox |
| | | | compartment. |
| T_{dist, sR1, peripheral} | Soluble receptor | 30 | The distribution rate of soluble |
| | 1 Tdist | | receptor 1 from the central |
| | peripheral | | compartment to the peripheral |
| | (hr) | | compartment. |
| T_{dist, sR2, peripheral} | Soluble receptor | 30 | The distribution rate of soluble |
| | 2 Tdist | | receptor 2 from the central |
| | peripheral | | compartment to the peripheral |
| | (hr) | | compartment. |
| T_{dist, sR1, disease} | Soluble receptor | 30 | The distribution rate of soluble |
| | 1 Tdist disease | | receptor 1 from the central |
| | (hr) | | compartment to the disease |
| | | | compartment. |
| T_{dist, sR2, disease} | Soluble receptor | 30 | The distribution rate of soluble |
| | 2 Tdist disease | | receptor 2 from the central |
| | (hr) | | compartment to the disease |
| | | | compartment. |
| T_{dist, sR1, tox} | Soluble receptor | 30 | The distribution rate of soluble |
| | 1 Tdist tox | | receptor 1 from the central |
| | (hr) | | compartment to the tox |
| | | | compartment. |
| T_{dist, sR2, tox} | Soluble receptor | 30 | The distribution rate of soluble |
| | 2 Tdist tox | | receptor 2 from the central |
| | (hr) | | compartment to the tox |
| | | | compartment. |
| Density_{cells, central} | Cell density | 1000000 | Density of receptor-bearing |
| | central | | cells in the central |
| | (#/mL) | | compartment. |
| Density_{cells, peripheral} | Cell density | 1000000 | Density of receptor-bearing |
| | peripheral | | cells in the peripheral |
| | (#/mL) | | compartment. |
| Density_{cells, disease} | Cell density | 1000000 | Density of receptor-bearing |
| | disease | | cells in the disease |
| | (#/mL) | | compartment. |
| Density_{cells, tox} | Cell density | 1000000 | Density of receptor-bearing |
| | tox | | cells in the tox compartment. |
| | (#/mL) | | |
| Scale_{KD, L1, central} | Drug: Target 1 | 1 | A multiplicative factor for the |
| | Kd scale | | equilibrium dissociation |
| | central | | constant between drug and |
| | | | ligand 1 in the central |
| | | | compartment. Can be used to |
| | | | model drugs whose target |
| | | | affinity is affected by the |
| | | | compartment. |
| Scale_{KD, L2, central} | Drug: Target 2 | 1 | A multiplicative factor for the |
| | Kd scale | | equilibrium dissociation |
| | central | | constant between drug and |
| | | | ligand 2 in the central |
| | | | compartment. Can be used to |
| | | | model drugs whose target |
| | | | affinity is affected by the |
| | | | compartment. |
| Scale_{KD, L1, peripheral} | Drug: Target 1 | 1 | A multiplicative factor for the |
| | Kd scale | | equilibrium dissociation |
| | peripheral | | constant between drug and |
| | | | ligand 1 in the peripheral |
| | | | compartment. Can be used to |
| | | | model drugs whose target |
| | | | affinity is affected by the |
| | | | compartment. |
| Scale_{KD, L2, peripheral} | Drug: Target 2 | 1 | A multiplicative factor for the |
| | Kd scale | | equilibrium dissociation |
| | peripheral | | constant between drug and |
| | | | ligand 2 in the peripheral |
| | | | compartment. Can be used to |
| | | | model drugs whose target |
| | | | affinity is affected by the |
| | | | compartment. |
| Scale_{KD, L1, disease} | Drug: Target 1 | 1 | A multiplicative factor for the |
| | Kd scale | | equilibrium dissociation |
| | disease | | constant between drug and |
| | | | ligand 1 in the disease |
| | | | compartment. Can be used to |
| | | | model drugs whose target |
| | | | affinity is affected by the |
| | | | compartment. |
| Scale_{KD, L2, disease} | Drug: Target 2 | 1 | A multiplicative factor for the |
| | Kd scale | | equilibrium dissociation |
| | disease | | constant between drug and |
| | | | ligand 2 in the disease |
| | | | compartment. Can be used to |
| | | | model drugs whose target |
| | | | affinity is affected by the |
| | | | compartment. |
| Scale_{KD, L1, tox} | Drug: Target 1 | 1 | A multiplicative factor for the |
| | Kd scale | | equilibrium dissociation |
| | tox | | constant between drug and |
| | | | ligand 1 in the tox |
| | | | compartment. Can be used to |
| | | | model drugs whose target |
| | | | affinity is affected by the |
| | | | compartment. |
| Scale_{KD, L2, tox} | Drug: Target 2 | 1 | A multiplicative factor for the |
| | Kd scale | | equilibrium dissociation |
| | tox | | constant between drug and |
| | | | ligand 2 in the tox |
| | | | compartment. Can be used to |
| | | | model drugs whose target |
| | | | affinity is affected by the |
| | | | compartment. |
| Scale_{t1/2, central} | Soluble Drug | 1 | A multiplicative factor for the |
| | T ½ scale | | drug elimination half-life in the |
| | central | | central compartment. Can be |
| | | | used to model drugs which |
| | | | have a different half-life in |
| | | | some compartments. |
| Scale_{t1/2, peripheral} | Soluble Drug | 1 | A multiplicative factor for the |
| | T ½ scale | | drug elimination half-life in the |
| | peripheral | | peripheral compartment. Can |
| | | | be used to model drugs which |
| | | | have a different half-life in |
| | | | some compartments. |
| Scale_{t1/2, disease} | Soluble Drug | 1 | A multiplicative factor for the |
| | T ½ scale | | drug elimination half-life in the |
| | disease | | disease compartment. Can be |
| | | | used to model drugs which |
| | | | have a different half-life in |
| | | | some compartments. |
| Scale_{t1/2, tox} | Soluble Drug | 1 | A multiplicative factor for the |
| | T ½ scale | | drug elimination half-life in the |
| | tox | | tox compartment. Can be used |
| | | | to model drugs which have a |
| | | | different half-life in some |
| | | | compartments. |

TABLE 5
Example target criteria (e.g., for the therapeutic drug candidate
and the target for the therapeutic purpose) of the bispecific anti-ligand ×
anti-ligand (4-compartment) example model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free target |
| Engagement | | | due to drug binding. |
| | | | Measured as a percent of untreated. |
| Activation | 90 | percent | Percent of receptor occupied |
| | | | by ligand or drug. |
| | | | Measured as a percent of total. |
| | | | This is typically used |
| | | | for agonists. |

TABLE 6
Example output plots of the bispecific anti-ligand ×
anti-ligand example model (4-compartment) in the EFA system.

| Output Plot | Description |
|---|---|
| Criterion | The value of the selected criterion |
| vs scan | for each value of the 1D scan |
| parameter | parameter simulated. The desired |
| | target value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is any form of the |
| | drug not bound to any membrane proteins. |
| | This may include drug bound to one or |
| | more soluble proteins such as |
| | ligands or soluble receptors. |

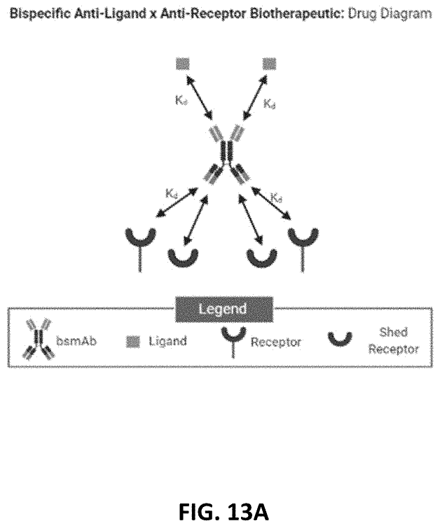

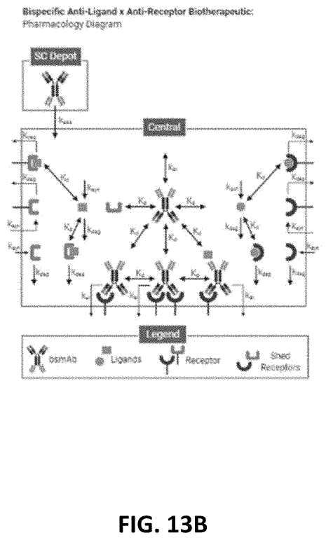

In some implementations, the example bispecific anti-ligand x anti-receptor model can include a one-compartment model of bivalent antibody binding to soluble ligand and membrane receptor A bispecific biotherapeutic that binds to a target soluble ligand and a target cell surface receptor. For the ligand the drug prevents the target ligand from binding to its cognate-receptor. For the receptor the drug either (1) acts as a competitive inhibitor by blocking the cognate-ligand from binding to its receptor or (2) acts as a receptor agonist. The molecule can be mono- or bivalent for each target. This is a one-compartment model and there is an option for +/− soluble receptor. FIGS. 13A-13B show schematic diagrams illustrating a biotherapeutic scenario of the example bispecific anti-ligand x anti-receptor model, according to some embodiments. Example parameters of the bispecific anti-ligand x anti-receptor model in the EFA system are shown in Table 7. Example target criteria (e.g., for the therapeutic drug candidate and the target for the therapeutic purpose) of the bispecific anti-ligand x anti-receptor example model in the EFA system are shown in Table 8. Example output plots of the bispecific anti-ligand x anti-receptor example model in the EFA system are shown in Table 9.

TABLE 7
Example parameters of the bispecific anti-ligand × anti-receptor model in the EFA system.

| Symbol | Name | Default | Definition |
|---|---|---|---|
| τ | Interval | 14 | The interval between doses of the drug. |
| D | Dose (mg) | 100 | The amount of each dose. |
| K_{D, L1} | Drug: Target | 0.1 | The affinity of the drug for ligand 1 as |
| | 1 KD (nM) | | characterized by the equilibrium |
| | | | dissociation constant. This is the |
| | | | monovalent affinity (i.e. the affinity of |
| | | | single binding). |
| K_{D, R2} | Drug: Target | 0.1 | The affinity of the drug for receptor 2 as |
| | 2 KD (nM) | | characterized by the equilibrium |
| | | | dissociation constant. This is the |
| | | | monovalent affinity (i.e. the affinity of |
| | | | single binding). |
| N_{doses} | Number of | 7 | The number of doses to be given. |
| | doses | | |
| MW | Molecular | 150000 | The molecular weight of the drug. This is |
| | Weight (Da) | | used to convert the dose which is |
| | | | expressed in mass units to a molar dose. |
| t_{1/2} | Biologic first | 28 | The first-order elimination rate of the drug. |
| | order T ½ | | |
| | (days) | | |
| t_{1/2, a} | SC absorption | 2.5 | The first-order absorption rate of the drug |
| | T ½ | | from the subcutaneous compartment into |
| | (days) | | the central compartment. |
| BW | Body | 70 | The body weight of the subject. This is |
| | Weight (kg) | | used to convert the dose when expressed in |
| | | | units of mg/kg. |
| V | Volume (L) | 5 | The volume of the central compartment |
| | | | which is typically the plasma of the |
| | | | peripheral blood. |
| Valency₁ | Effective | 2 | The number of ligand 1 molecules that a |
| | Valency 1 | | molecule of drug can bind. This may be 1 |
| | | | or 2. |
| Valency₂ | Effective | 2 | The number of receptor 2 molecules that a |
| | Valency 2 | | molecule of drug can bind. This may be 1 |
| | | | or 2. |
| t_{1/2, L1} | Ligand 1 | 0.5 | The first-order elimination rate of ligand 1 |
| | T ½ (hr) | | from the central compartment. |
| t_{1/2, L2} | Ligand 2 | 0.5 | The first-order elimination rate of ligand 2 |
| | T ½ (hr) | | from the central compartment. |
| t_{1/2, R1} | Receptor 1 | 1 | The first-order turnover rate of receptor 1 |
| | T ½ (hr) | | in all compartments. |
| t_{1/2, R2} | Receptor 2 | 1 | The first-order turnover rate of receptor 2 |
| | T ½ (hr) | | in all compartments. |
| t_{1/2, sR1} | Soluble | 0.5 | The first-order elimination rate of soluble |
| | receptor 1 | | receptor 1 from the central compartment. |
| | T ½ (hr) | | |
| t_{1/2, sR2} | Soluble | 0.5 | The first-order elimination rate of soluble |
| | receptor 2 | | receptor 2 from the central compartment. |
| | T ½ (hr) | | |
| K_{D, L1:R1} | L1:R1 | 1 | The binding affinity of ligand 1 for |
| | Affinity (nM) | | receptor 1 as characterized by the |
| | | | equilibrium dissociation constant. |
| K_{D, L2:R2} | L2:R2 | 1 | The binding affinity of ligand 2 for |
| | Affinity (nM) | | receptor 2 as characterized by the |
| | | | equilibrium dissociation constant. |
| C_{SS, L1} | Ligand 1 | 0.05 | The steady-state concentration of free |
| | CSS (nM) | | ligand 1 in the compartment in the absence |
| | | | of drug. Ligand 1 bound to receptor 1 is |
| | | | not included in this quantity. |
| C_{SS, L2} | Ligand 2 | 0.05 | The steady-state concentration of free |
| | CSS (nM) | | ligand 2 in the compartment in the absence |
| | | | of drug. Ligand 2 bound to receptor 2 is |
| | | | not included in this quantity. |
| C_{SS, R1} | Receptor 1 | 10000 | The steady-state concentration of receptor |
| | CSS | | 1 in the compartment in the absence of |
| | (#/cell) | | drug. Receptor 1 bound to ligand 1 is |
| | | | included in this quantity, but soluble |
| | | | receptor 1 is not. |
| C_{SS, R2} | Receptor 2 | 10000 | The steady-state concentration of receptor |
| | CSS | | 2 in the compartment in the absence of |
| | (#/cell) | | drug. Receptor 2 bound to ligand 2 is |
| | | | included in this quantity, but soluble |
| | | | receptor 2 is not. |
| C_{SS, sR1} | Soluble | 0 | The steady-state concentration of soluble |
| | receptor 1 | | receptor 1 in the compartment in the |
| | CSS (nM) | | absence of drug. |
| C_{SS, sR2} | Soluble | 0 | The steady-state concentration of soluble |
| | receptor 2 | | receptor 2 in the compartment in the |
| | CSS (nM) | | absence of drug. |
| Density_{cells} | Cell density | 1000000 | Density of receptor-bearing cells in the |
| | (#/mL) | | compartment. |
| Scale_{t1/2, R2, D:R2} | Drug: Receptor 2 | 1 | A multiplicative factor for receptor 2 |
| | T ½ | | turnover half-life when drug is bound to it |
| | scale | | in the compartment. Can be used to model |
| | | | effects the drug has on the internalization |
| | | | rate of this target. |
| Scale_{KD, L1} | Drug: Target 1 | 1 | A multiplicative factor for the equilibrium |
| | Kd scale | | dissociation constant between drug and |
| | | | ligand 1 in the compartment. Can be used |
| | | | to model drugs whose target affinity is |
| | | | affected by the compartment. |
| Scale_{KD, R2} | Drug: Target 2 | 1 | A multiplicative factor for the equilibrium |
| | Kd scale | | dissociation constant between drug and |
| | | | receptor 2 in the compartment. Can be |
| | | | used to model drugs whose target affinity |
| | | | is affected by the compartment. |

TABLE 8
Example target criteria (e.g., for the therapeutic drug candidate
and the target for the therapeutic purpose) of the bispecific
anti-ligand × anti- receptor example model in the EFA system.

| | Default | | |
|---|---|---|---|
| Criterion | Target | Units | Description |
| Inhibition | 90 | percent | Inhibition of ligand receptor complex. |
| | | | Measured as a percent of untreated. |
| Target | 90 | percent | Percent reduction of free |
| Engagement | | | target due to drug |
| | | | binding. Measured as a |
| | | | percent of untreated. |
| Activation | 90 | percent | Percent of receptor occupied |
| | | | by ligand or drug. |
| | | | Measured as a percent of total. |
| | | | This is typically |
| | | | used for agonists. |

TABLE 9
Example output plots of the bispecific anti-ligand ×
anti- receptor example model in the EFA system.

| Output Plot | Description |
|---|---|
| Criterion | The value of the selected criterion |
| vs scan | for each value of the 1D scan |
| parameter | parameter simulated. The desired target |
| | value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is any form of the |
| | drug not bound to any membrane proteins. |
| | This may include drug bound to one or |
| | more soluble proteins such as |
| | ligands or soluble receptors. |

In some implementations, the example bispecific anti-ligand x anti-receptor (4-compartment) includes a four-compartment model of bivalent antibody binding to soluble ligand and membrane receptor. A bispecific biotherapeutic that binds to a target soluble ligand and a target cell surface receptor. For the ligand the drug can prevent the target ligand from binding to its cognate-receptor. For the receptor the drug can either (1) act as a competitive inhibitor by blocking the cognate-ligand from binding to its receptor or (2) act as a receptor agonist. The molecule can be mono- or bivalent for each target. This is a four-compartment model with a central, peripheral, disease and tox compartments. There is an option for +/− soluble receptor for each receptor. FIG. 13A shows a schematic diagram illustrating a biotherapeutic scenario of the example bispecific anti-ligand x anti-receptor (4-compartment) model, according to some embodiments. Example parameters of the bispecific anti-ligand x anti-receptor (4-compartment) example model in the EFA system are shown in Table 10. Example target criteria (e.g., for the therapeutic drug candidate and the target for the therapeutic purpose) of the bispecific anti-ligand x anti-receptor (4-compartment) model in the EFA system are shown in Table 11. Example output plots of the example bispecific anti-ligand x anti-receptor (4-compartment) model in the EFA system are shown in Table 12.

TABLE 10
Example parameters of the bispecific anti-ligand ×
anti-receptor (4-compartment) example model in the EFA system.

| Symbol | Name | Default | Definition |
|---|---|---|---|
| τ | Interval | 14 | The interval between doses of the |
| | | | drug. |
| D | Dose (mg) | 100 | The amount of each dose. |
| K_{D, L1} | Drug: Target | 0.1 | The affinity of the drug for ligand 1 |
| | 1 KD (nM) | | as characterized by the equilibrium |
| | | | dissociation constant. This is the |
| | | | monovalent affinity (i.e. the affinity |
| | | | of single binding). |
| K_{D, R2} | Drug: Target | 0.1 | The affinity of the drug for receptor |
| | 2 KD (nM) | | 2 as characterized by the |
| | | | equilibrium dissociation constant. |
| | | | This is the monovalent affinity (i.e. |
| | | | the affinity of single binding). |
| N_{doses} | Number of | 7 | The number of doses to be given. |
| | doses | | |
| MW | Molecular | 150000 | The molecular weight of the drug. |
| | Weight (Da) | | This is used to convert the dose |
| | | | which is expressed in mass units to |
| | | | a molar dose. |
| t_{1/2} | Biologic first | 28 | The first-order elimination rate of |
| | order T ½ | | the drug. |
| | (days) | | |
| t_{1/2, a} | SC absorption | 2.5 | The first-order absorption rate of the |
| | T ½ (days) | | drug from the subcutaneous |
| | | | compartment into the central |
| | | | compartment. |
| BW | Body | 70 | The body weight of the subject. This |
| | Weight (kg) | | is used to convert the dose when |
| | | | expressed in units of mg/kg. |
| V | Volume | 2.5 | The volume of the central |
| | central (L) | | compartment which is typically the |
| | | | plasma of the peripheral blood. |
| Vₚₑᵣᵢₚₕₑᵣₐₗ | Volume | 12.8 | The volume of the non-blood fluids |
| | peripheral (L) | | that the antibody can distribute to. |
| V_{disease} | Volume | 0.1 | The volume of the disease |
| | disease (L) | | compartment interstitial fluid. |
| Vₜₒₓ | Volume | 0.1 | The volume of the tox compartment |
| | tox (L) | | interstitial fluid. |
| T_{dist, peripheral} | Drug Tdist | 30 | The distribution rate of drug from |
| | peripheral | | the central compartment to the |
| | (hr) | | peripheral compartment. |
| T_{dist, disease} | Drug Tdist | 30 | The distribution rate of drug from |
| | disease (hr) | | the central compartment to the |
| | | | disease compartment. |
| T_{dist, tox} | Drug Tdist | 30 | The distribution rate of drug from |
| | tox (hr) | | the central compartment to the tox |
| | | | compartment. |
| P_{dist, peripheral} | Drug partition | 0.190625 | The steady-state ratio of soluble |
| | coefficient | | drug in the peripheral compartment |
| | peripheral | | to soluble drug in the central |
| | | | compartment. |
| P_{dist, disease} | Drug partition | 0.3 | The steady-state ratio of soluble |
| | coefficient | | drug in the disease compartment to |
| | disease | | soluble drug in the central |
| | | | compartment. |
| P_{dist, tox} | Drug partition | 0.3 | The steady-state ratio of soluble |
| | coefficient | | drug in the tox compartment to |
| | tox | | soluble drug in the central |
| | | | compartment. |
| Valency₁ | Effective | 2 | The number of ligand 1 molecules |
| | Valency 1 | | that a molecule of drug can bind. |
| | | | This may be 1 or 2. |
| Valency₂ | Effective | 2 | The number of receptor 2 molecules |
| | Valency 2 | | that a molecule of drug can bind. |
| | | | This may be 1 or 2. |
| t_{1/2, L1} | Ligand 1 | 0.5 | The first-order elimination rate of |
| | T ½ (hr) | | ligand 1 from the central |
| | | | compartment. |
| t_{1/2, L2} | Ligand 2 | 0.5 | The first-order elimination rate of |
| | T ½ (hr) | | ligand 2 from the central |
| | | | compartment. |
| t_{1/2, R1} | Receptor 1 | 1 | The first-order turnover rate of |
| | T ½ (hr) | | receptor 1 in all compartments. |
| t_{1/2, R2} | Receptor 2 | 1 | The first-order turnover rate of |
| | T ½ (hr) | | receptor 2 in all compartments. |
| t_{1/2, sR1} | Soluble | 0.5 | The first-order elimination rate of |
| | receptor 1 | | soluble receptor 1 from the central |
| | T ½ (hr) | | compartment. |
| t_{1/2, sR2} | Soluble | 0.5 | The first-order elimination rate of |
| | receptor 2 | | soluble receptor 2 from the central |
| | T ½ (hr) | | compartment. |
| K_{D, L1:R1} | L1:R1 | 1 | The binding affinity of ligand 1 for |
| | Affinity (nM) | | receptor 1 as characterized by the |
| | | | equilibrium dissociation constant. |
| K_{D, L2:R2} | L2:R2 | 1 | The binding affinity of ligand 2 for |
| | Affinity (nM) | | receptor 2 as characterized by the |
| | | | equilibrium dissociation constant. |
| C_{SS, L1, central} | Ligand 1 CSS | 0.05 | The steady-state concentration of |
| | central (nM) | | free ligand 1 in the central |
| | | | compartment in the absence of drug. |
| | | | Ligand 1 bound to receptor 1 is not |
| | | | included in this quantity. |
| C_{SS, L2, central} | Ligand 2 CSS | 0.05 | The steady-state concentration of |
| | central (nM) | | free ligand 2 in the central |
| | | | compartment in the absence of drug. |
| | | | Ligand 2 bound to receptor 2 is not |
| | | | included in this quantity. |
| C_{SS, L1, peripheral} | Ligand 1 CSS | 0.05 | The steady-state concentration of |
| | peripheral (nM) | | free ligand 1 in the peripheral |
| | | | compartment in the absence of drug. |
| | | | Ligand 1 bound to receptor 1 is not |
| | | | included in this quantity. |
| C_{SS, L2, peripheral} | Ligand 2 CSS | 0.05 | The steady-state concentration of |
| | peripheral (nM) | | free ligand 2 in the peripheral |
| | | | compartment in the absence of drug. |
| | | | Ligand 2 bound to receptor 2 is not |
| | | | included in this quantity. |
| C_{SS, L1, disease} | Ligand 1 CSS | 0.05 | The steady-state concentration of |
| | disease (nM) | | free ligand 1 in the disease |
| | | | compartment in the absence of drug. |
| | | | Ligand 1 bound to receptor 1 is not |
| | | | included in this quantity. |
| C_{SS, L2, disease} | Ligand 2 CSS | 0.05 | The steady-state concentration of |
| | disease (nM) | | free ligand 2 in the disease |
| | | | compartment in the absence of drug. |
| | | | Ligand 2 bound to receptor 2 is not |
| | | | included in this quantity. |
| C_{SS, L1, tox} | Ligand 1 CSS | 0.05 | The steady-state concentration of |
| | tox (nM) | | free ligand 1 in the tox compartment |
| | | | in the absence of drug. Ligand 1 |
| | | | bound to receptor 1 is not included |
| | | | in this quantity. |
| C_{SS, L2, tox} | Ligand 2 CSS | 0.05 | The steady-state concentration of |
| | tox (nM) | | free ligand 2 in the tox compartment |
| | | | in the absence of drug. Ligand 2 |
| | | | bound to receptor 2 is not included |
| | | | in this quantity. |
| C_{SS, R1, central} | Receptor 1 | 10000 | The steady-state concentration of |
| | CSS central | | receptor 1 in the central |
| | (#/cell) | | compartment in the absence of drug. |
| | | | Receptor 1 bound to ligand 1 is |
| | | | included in this quantity, but soluble |
| | | | receptor 1 is not. |
| C_{SS, R2, central} | Receptor 2 | 10000 | The steady-state concentration of |
| | CSS central | | receptor 2 in the central |
| | (#/cell) | | compartment in the absence of drug. |
| | | | Receptor 2 bound to ligand 2 is |
| | | | included in this quantity, but soluble |
| | | | receptor 2 is not. |
| C_{SS, R1, peripheral} | Receptor 1 | 10000 | The steady-state concentration of |
| | CSS peripheral | | receptor 1 in the peripheral |
| | (#/cell) | | compartment in the absence of drug. |
| | | | Receptor 1 bound to ligand 1 is |
| | | | included in this quantity, but soluble |
| | | | receptor 1 is not. |
| C_{SS, R2, peripheral} | Receptor 2 | 10000 | The steady-state concentration of |
| | CSS peripheral | | receptor 2 in the peripheral |
| | (#/cell) | | compartment in the absence of drug. |
| | | | Receptor 2 bound to ligand 2 is |
| | | | included in this quantity, but soluble |
| | | | receptor 2 is not. |
| C_{SS, R1, disease} | Receptor 1 | 10000 | The steady-state concentration of |
| | CSS disease | | receptor 1 in the disease |
| | (#/cell) | | compartment in the absence of drug. |
| | | | Receptor 1 bound to ligand 1 is |
| | | | included in this quantity, but soluble |
| | | | receptor 1 is not. |
| C_{SS, R2, disease} | Receptor 2 | 10000 | The steady-state concentration of |
| | CSS disease | | receptor 2 in the disease |
| | (#/cell) | | compartment in the absence of drug. |
| | | | Receptor 2 bound to ligand 2 is |
| | | | included in this quantity, but soluble |
| | | | receptor 2 is not. |
| C_{SS, R1, tox} | Receptor 1 | 10000 | The steady-state concentration of |
| | CSS tox | | receptor 1 in the tox compartment in |
| | (#/cell) | | the absence of drug. Receptor 1 |
| | | | bound to ligand 1 is included in this |
| | | | quantity, but soluble receptor 1 is |
| | | | not. |
| C_{SS, R2, tox} | Receptor 2 | 10000 | The steady-state concentration of |
| | CSS tox | | receptor 2 in the tox compartment in |
| | (#/cell) | | the absence of drug. Receptor 2 |
| | | | bound to ligand 2 is included in this |
| | | | quantity, but soluble receptor 2 is |
| | | | not. |
| C_{SS, sR1, central} | Soluble | 0 | The steady-state concentration of |
| | receptor 1 CSS | | soluble receptor 1 in the central |
| | central (nM) | | compartment in the absence of drug. |
| C_{SS, sR2, central} | Soluble | 0 | The steady-state concentration of |
| | receptor 2 CSS | | soluble receptor 2 in the central |
| | central (nM) | | compartment in the absence of drug. |
| C_{SS, sR1, peripheral} | Soluble | 0 | The steady-state concentration of |
| | receptor 1 CSS | | soluble receptor 1 in the peripheral |
| | peripheral (nM) | | compartment in the absence of drug. |
| C_{SS, sR2, peripheral} | Soluble | 0 | The steady-state concentration of |
| | receptor 2 CSS | | soluble receptor 2 in the peripheral |
| | peripheral (nM) | | compartment in the absence of drug. |
| C_{SS, sR1, disease} | Soluble | 0 | The steady-state concentration of |
| | receptor 1 CSS | | soluble receptor 1 in the disease |
| | disease (nM) | | compartment in the absence of drug. |
| C_{SS, sR2, disease} | Soluble | 0 | The steady-state concentration of |
| | receptor 2 CSS | | soluble receptor 2 in the disease |
| | disease (nM) | | compartment in the absence of drug. |
| C_{SS, sR1, tox} | Soluble | 0 | The steady-state concentration of |
| | receptor 1 CSS | | soluble receptor 1 in the tox |
| | tox (nM) | | compartment in the absence of drug. |
| C_{SS, sR2, tox} | Soluble receptor | 0 | The steady-state concentration of |
| | 2 CSS tox | | soluble receptor 2 in the tox |
| | (nM) | | compartment in the absence of drug. |
| T_{dist, L1, peripheral} | Ligand 1 Tdist | 30 | The distribution rate of ligand 1 |
| | peripheral | | from the central compartment to the |
| | (hr) | | peripheral compartment. |
| T_{dist, L2, peripheral} | Ligand 2 Tdist | 30 | The distribution rate of ligand 2 |
| | peripheral | | from the central compartment to the |
| | (hr) | | peripheral compartment. |
| T_{dist, L1, disease} | Ligand 1 Tdist | 30 | The distribution rate of ligand 1 |
| | disease (hr) | | from the central compartment to the |
| | | | disease compartment. |
| T_{dist, L2, disease} | Ligand 2 Tdist | 30 | The distribution rate of ligand 2 |
| | disease (hr) | | from the central compartment to the |
| | | | disease compartment. |
| T_{dist, L1, tox} | Ligand 1 Tdist | 30 | The distribution rate of ligand 1 |
| | tox (hr) | | from the central compartment to the |
| | | | tox compartment. |
| T_{dist, L2, tox} | Ligand 2 Tdist | 30 | The distribution rate of ligand 2 |
| | tox (hr) | | from the central compartment to the |
| | | | tox compartment. |
| T_{dist, sR1, peripheral} | Soluble receptor | 30 | The distribution rate of soluble |
| | 1 Tdist | | receptor 1 from the central |
| | peripheral (hr) | | compartment to the peripheral |
| | | | compartment. |
| T_{dist, sR2, peripheral} | Soluble receptor | 30 | The distribution rate of soluble |
| | 2 Tdist | | receptor 2 from the central |
| | peripheral (hr) | | compartment to the peripheral |
| | | | compartment. |
| T_{dist, sR1, disease} | Soluble receptor | 30 | The distribution rate of soluble |
| | 1 Tdist | | receptor 1 from the central |
| | disease (hr) | | compartment to the disease |
| | | | compartment. |
| T_{dist, sR2, disease} | Soluble receptor | 30 | The distribution rate of soluble |
| | 2 Tdist | | receptor 2 from the central |
| | disease (hr) | | compartment to the disease |
| | | | compartment. |
| T_{dist, sR1, tox} | Soluble receptor | 30 | The distribution rate of soluble |
| | 1 Tdist tox | | receptor 1 from the central |
| | (hr) | | compartment to the tox |
| | | | compartment. |
| T_{dist, sR2, tox} | Soluble receptor | 30 | The distribution rate of soluble |
| | 2 Tdist tox | | receptor 2 from the central |
| | (hr) | | compartment to the tox |
| | | | compartment. |
| Density_{cells, central} | Cell density | 1000000 | Density of receptor-bearing cells in |
| | central | | the central compartment. |
| | (#/mL) | | |
| Density_{cells, peripheral} | Cell density | 1000000 | Density of receptor-bearing cells in |
| | peripheral | | the peripheral compartment. |
| | (#/mL) | | |
| Density_{cells, disease} | Cell density | 1000000 | Density of receptor-bearing cells in |
| | disease | | the disease compartment. |
| | (#/mL) | | |
| Density_{cells, tox} | Cell density | 1000000 | Density of receptor-bearing cells in |
| | tox (#/mL) | | the tox compartment. |
| Scale_{t1/2, R2, D:R2, central} | Drug: Receptor 2 | 1 | A multiplicative factor for receptor |
| | T ½ scale | | 2 turnover half-life when drug is |
| | central | | bound to it in the central |
| | | | compartment. Can be used to model |
| | | | effects the drug has on the |
| | | | internalization rate of this target. |
| Scale_{t1/2, R2, D:R2, peripheral} | Drug: Receptor 2 | 1 | A multiplicative factor for receptor |
| | T ½ scale | | 2 turnover half-life when drug is |
| | peripheral | | bound to it in the peripheral |
| | | | compartment. Can be used to model |
| | | | effects the drug has on the |
| | | | internalization rate of this target. |
| Scale_{t1/2, R2, D:R2, disease} | Drug: Receptor 2 | 1 | A multiplicative factor for receptor |
| | T ½ scale | | 2 turnover half-life when drug is |
| | disease | | bound to it in the disease |
| | | | compartment. Can be used to model |
| | | | effects the drug has on the |
| | | | internalization rate of this target. |
| Scale_{t1/2, R2, D:R2, tox} | Drug: Receptor 2 | 1 | A multiplicative factor for receptor |
| | T ½ scale | | 2 turnover half-life when drug is |
| | tox | | bound to it in the tox compartment. |
| | | | Can be used to model effects the |
| | | | drug has on the internalization rate |
| | | | of this target. |
| Scale_{KD, L1, central} | Drug: Target 1 | 1 | A multiplicative factor for the |
| | Kd scale | | equilibrium dissociation constant |
| | central | | between drug and ligand 1 in the |
| | | | central compartment. Can be used to |
| | | | model drugs whose target affinity is |
| | | | affected by the compartment. |
| Scale_{KD, R2, central} | Drug: Target 2 | 1 | A multiplicative factor for the |
| | Kd scale | | equilibrium dissociation constant |
| | central | | between drug and receptor 2 in the |
| | | | central compartment. Can be used to |
| | | | model drugs whose target affinity is |
| | | | affected by the compartment. |
| Scale_{KD, L1, peripheral} | Drug: Target 1 | 1 | A multiplicative factor for the |
| | Kd scale | | equilibrium dissociation constant |
| | peripheral | | between drug and ligand 1 in the |
| | | | peripheral compartment. Can be |
| | | | used to model drugs whose target |
| | | | affinity is affected by the |
| | | | compartment. |
| Scale_{KD, R2, peripheral} | Drug: Target 2 | 1 | A multiplicative factor for the |
| | Kd scale | | equilibrium dissociation constant |
| | peripheral | | between drug and receptor 2 in the |
| | | | peripheral compartment. Can be |
| | | | used to model drugs whose target |
| | | | affinity is affected by the |
| | | | compartment. |
| Scale_{KD, L1, disease} | Drug: Target 1 | 1 | A multiplicative factor for the |
| | Kd scale | | equilibrium dissociation constant |
| | disease | | between drug and ligand 1 in the |
| | | | disease compartment. Can be used |
| | | | to model drugs whose target affinity |
| | | | is affected by the compartment. |
| Scale_{KD, R2, disease} | Drug: Target 2 | 1 | A multiplicative factor for the |
| | Kd scale | | equilibrium dissociation constant |
| | disease | | between drug and receptor 2 in the |
| | | | disease compartment. Can be used |
| | | | to model drugs whose target affinity |
| | | | is affected by the compartment. |
| Scale_{KD, L1, tox} | Drug: Target 1 | 1 | A multiplicative factor for the |
| | Kd scale | | equilibrium dissociation constant |
| | tox | | between drug and ligand 1 in the tox |
| | | | compartment. Can be used to model |
| | | | drugs whose target affinity is |
| | | | affected by the compartment. |
| Scale_{KD, R2, tox} | Drug: Target 2 | 1 | A multiplicative factor for the |
| | Kd scale | | equilibrium dissociation constant |
| | tox | | between drug and receptor 2 in the |
| | | | tox compartment. Can be used to |
| | | | model drugs whose target affinity is |
| | | | affected by the compartment. |
| Scale_{t1/2, central} | Soluble Drug | 1 | A multiplicative factor for the drug |
| | T ½ scale | | elimination half-life in the central |
| | central | | compartment. Can be used to model |
| | | | drugs which have a different half- |
| | | | life in some compartments. |
| Scale_{t1/2, peripheral} | Soluble Drug | 1 | A multiplicative factor for the drug |
| | T ½ scale | | elimination half-life in the |
| | peripheral | | peripheral compartment. Can be |
| | | | used to model drugs which have a |
| | | | different half-life in some |
| | | | compartments. |
| Scale_{t1/2, disease} | Soluble Drug | 1 | A multiplicative factor for the drug |
| | T ½ scale | | elimination half-life in the disease |
| | disease | | compartment. Can be used to model |
| | | | drugs which have a different half- |
| | | | life in some compartments. |
| Scale_{t1/2, tox} | Soluble Drug | 1 | A multiplicative factor for the drug |
| | T ½ scale | | elimination half-life in the tox |
| | tox | | compartment. Can be used to model |
| | | | drugs which have a different half- |
| | | | life in some compartments. |

TABLE 11
Example target criteria (e.g., for the therapeutic drug candidate
and the target for the therapeutic purpose) of the bispecific anti-
ligand × anti-receptor (4-compartment) model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free |
| Engagement | | | target due to drug binding. |
| | | | Measured as a percent of untreated. |
| Activation | 90 | percent | Percent of receptor occupied |
| | | | by ligand or drug. |
| | | | Measured as a percent of total. |
| | | | This is typically used |
| | | | for agonists. |

TABLE 12
Example output plots of the example bispecific anti-ligand ×
anti-receptor (4-compartment) model in the EFA system.

| Output Plot | Description |
|---|---|
| Criterion | The value of the selected criterion |
| vs scan | for each value of the 1D scan |
| parameter | parameter simulated. The desired target |
| | value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is |
| | any form of the drug not bound to |
| | any membrane proteins. This |
| | may include drug bound to one or |
| | more soluble proteins such as |
| | ligands or soluble receptors. |

In some implementations, the example bispecific anti-ligand x anti-receptor with avidity (4-compartment) model can include a four-compartment model of avid bivalent antibody binding to soluble ligand and membrane receptor. A bispecific biotherapeutic that binds to a target soluble ligand and a target cell surface receptor. For the ligand the drug prevents the target ligand from binding to its cognate-receptor. For the receptor the drug either (1) acts as a competitive inhibitor by blocking the cognate-ligand from binding to its receptor or (2) acts as a receptor agonist. The molecule can be monovalent or bivalent for each target with avidity for the receptor target. This is a four-compartment model with a central, peripheral, disease and tox compartments. There is an option for +/− soluble receptor for each receptor. FIG. 13A shows a schematic diagram illustrating a biotherapeutic scenario of the example bispecific anti-ligand x anti-receptor with avidity (4-compartment) model, according to some embodiments. Example parameters of the bispecific anti-ligand x anti-receptor with avidity (4-compartment) model in the EFA system are shown in Table 13. Example target criteria (e.g., for the therapeutic drug candidate and the target for the therapeutic purpose) of the bispecific anti-ligand x anti-receptor with avidity (4-compartment) example model in the EFA system are shown in Table 14. Example output plots of the bispecific anti-ligand x anti-receptor with avidity (4-compartment) example model in the EFA system are shown in Table 15.

TABLE 13
Example parameters of the bispecific anti-ligand × anti-
receptor with avidity (4-compartment) model in the EFA system.

| Symbol | Name | Default | Definition |
|---|---|---|---|
| τ | Interval | 14 | The interval between doses of the |
| | | | drug. |
| D | Dose (mg) | 100 | The amount of each dose. |
| K_{D, L1} | Drug: Target | 0.1 | The affinity of the drug for ligand 1 |
| | 1 KD (nM) | | as characterized by the equilibrium |
| | | | dissociation constant. This is the |
| | | | monovalent affinity (i.e. the affinity |
| | | | of single binding). |
| K_{D, R2} | Drug: Target | 0.1 | The affinity of the drug for receptor |
| | 2 KD (nM) | | 2 as characterized by the |
| | | | equilibrium dissociation constant. |
| | | | This is the monovalent affinity (i.e. |
| | | | the affinity of single binding). |
| N_{doses} | Number of doses | 7 | The number of doses to be given. |
| MW | Molecular | 150000 | The molecular weight of the drug. |
| | Weight (Da) | | This is used to convert the dose |
| | | | which is expressed in mass units to |
| | | | a molar dose. |
| t_{1/2} | Biologic first | 28 | The first-order elimination rate of |
| | order T ½ (days) | | the drug. |
| t_{1/2, a} | SC absorption | 2.5 | The first-order absorption rate of |
| | T ½ (days) | | the drug from the subcutaneous |
| | | | compartment into the central |
| | | | compartment. |
| BW | Body | 70 | The body weight of the subject. |
| | Weight (kg) | | This is used to convert the dose |
| | | | when expressed in units of mg/kg. |
| V | Volume | 2.5 | The volume of the central |
| | central (L) | | compartment which is typically the |
| | | | plasma of the peripheral blood. |
| Vₚₑᵣᵢₚₕₑᵣₐₗ | Volume | 12.8 | The volume of the non-blood fluids |
| | peripheral (L) | | that the antibody can distribute to. |
| V_{disease} | Volume | 0.1 | The volume of the disease |
| | disease (L) | | compartment interstitial fluid. |
| Vₜₒₓ | Volume | 0.1 | The volume of the tox compartment |
| | tox (L) | | interstitial fluid. |
| T_{dist, peripheral} | Drug Tdist | 30 | The distribution rate of drug from |
| | peripheral (hr) | | the central compartment to the |
| | | | peripheral compartment. |
| T_{dist, disease} | Drug Tdist | 30 | The distribution rate of drug from |
| | disease (hr) | | the central compartment to the |
| | | | disease compartment. |
| T_{dist, tox} | Drug Tdist | 30 | The distribution rate of drug from |
| | tox (hr) | | the central compartment to the tox |
| | | | compartment. |
| P_{dist, peripheral} | Drug partition | 0.190625 | The steady-state ratio of soluble |
| | coefficient | | drug in the peripheral compartment |
| | peripheral | | to soluble drug in the central |
| | | | compartment. |
| P_{dist, disease} | Drug partition | 0.3 | The steady-state ratio of soluble |
| | coefficient | | drug in the disease compartment to |
| | disease | | soluble drug in the central |
| | | | compartment. |
| P_{dist, tox} | Drug partition | 0.3 | The steady-state ratio of soluble |
| | coefficient tox | | drug in the tox compartment to |
| | | | soluble drug in the central |
| | | | compartment. |
| Valency₁ | Effective | 2 | The number of ligand 1 molecules |
| | Valency 1 | | that a molecule of drug can bind. |
| | | | This may be 1 or 2. |
| Valency₂ | Effective | 2 | The number of receptor 2 molecules |
| | Valency 2 | | that a molecule of drug can bind. |
| | | | This may be 1 or 2. |
| χ° | Avidity | 1 | The chi factor of a standard assay. It |
| | chi factor | | is the observed chi factor of an |
| | | | assay with with 1e6 cells/mL with a |
| | | | cell diameter of 10 um. |
| t_{1/2, L1} | Ligand 1 | 0.5 | The first-order elimination rate of |
| | T ½ (hr) | | ligand 1 from the central |
| | | | compartment. |
| t_{1/2, L2} | Ligand 2 | 0.5 | The first-order elimination rate of |
| | T ½ (hr) | | ligand 2 from the central |
| | | | compartment. |
| t_{1/2, R1} | Receptor 1 | 1 | The first-order turnover rate of |
| | T ½ (hr) | | receptor 1 in all compartments. |
| t_{1/2, R2} | Receptor 2 | 1 | The first-order turnover rate of |
| | T ½ (hr) | | receptor 2 in all compartments. |
| t_{1/2, sR1} | Soluble receptor | 0.5 | The first-order elimination rate of |
| | 1 T ½ (hr) | | soluble receptor 1 from the central |
| | | | compartment. |
| t_{1/2, sR2} | Soluble receptor | 0.5 | The first-order elimination rate of |
| | 2 T ½ (hr) | | soluble receptor 2 from the central |
| | | | compartment. |
| K_{D, L1:R1} | L1:R1 | 1 | The binding affinity of ligand 1 for |
| | Affinity (nM) | | receptor 1 as characterized by the |
| | | | equilibrium dissociation constant. |
| K_{D, L2:R2} | L2:R2 | 1 | The binding affinity of ligand 2 for |
| | Affinity (nM) | | receptor 2 as characterized by the |
| | | | equilibrium dissociation constant. |
| C_{SS, L1, central} | Ligand 1 CSS | 0.05 | The steady-state concentration of |
| | central (nM) | | free ligand 1 in the central |
| | | | compartment in the absence of |
| | | | drug. Ligand 1 bound to receptor 1 |
| | | | is not included in this quantity. |
| C_{SS, L2, central} | Ligand 2 CSS | 0.05 | The steady-state concentration of |
| | central (nM) | | free ligand 2 in the central |
| | | | compartment in the absence of |
| | | | drug. Ligand 2 bound to receptor 2 |
| | | | is not included in this quantity. |
| C_{SS, L1, peripheral} | Ligand 1 CSS | 0.05 | The steady-state concentration of |
| | peripheral (nM) | | free ligand 1 in the peripheral |
| | | | compartment in the absence of |
| | | | drug. Ligand 1 bound to receptor 1 |
| | | | is not included in this quantity. |
| C_{SS, L2, peripheral} | Ligand 2 CSS | 0.05 | The steady-state concentration of |
| | peripheral (nM) | | free ligand 2 in the peripheral |
| | | | compartment in the absence of |
| | | | drug. Ligand 2 bound to receptor 2 |
| | | | is not included in this quantity. |
| C_{SS, L1, disease} | Ligand 1 CSS | 0.05 | The steady-state concentration of |
| | disease (nM) | | free ligand 1 in the disease |
| | | | compartment in the absence of |
| | | | drug. Ligand 1 bound to receptor 1 |
| | | | is not included in this quantity. |
| C_{SS, L2, disease} | Ligand 2 CSS | 0.05 | The steady-state concentration of |
| | disease (nM) | | free ligand 2 in the disease |
| | | | compartment in the absence of |
| | | | drug. Ligand 2 bound to receptor 2 |
| | | | is not included in this quantity. |
| C_{SS, L1, tox} | Ligand 1 CSS | 0.05 | The steady-state concentration of |
| | tox (nM) | | free ligand 1 in the tox |
| | | | compartment in the absence of |
| | | | drug. Ligand 1 bound to receptor 1 |
| | | | is not included in this quantity. |
| C_{SS, L2, tox} | Ligand 2 CSS | 0.05 | The steady-state concentration of |
| | tox (nM) | | free ligand 2 in the tox |
| | | | compartment in the absence of |
| | | | drug. Ligand 2 bound to receptor 2 |
| | | | is not included in this quantity. |
| C_{SS, R1, central} | Receptor 1 | 10000 | The steady-state concentration of |
| | CSS central | | receptor 1 in the central |
| | (#/cell) | | compartment in the absence of |
| | | | drug. Receptor 1 bound to ligand 1 |
| | | | is included in this quantity, but |
| | | | soluble receptor 1 is not. |
| C_{SS, R2, central} | Receptor 2 | 10000 | The steady-state concentration of |
| | CSS central | | receptor 2 in the central |
| | (#/cell) | | compartment in the absence of |
| | | | drug. Receptor 2 bound to ligand 2 |
| | | | is included in this quantity, but |
| | | | soluble receptor 2 is not. |
| C_{SS, R1, peripheral} | Receptor 1 | 10000 | The steady-state concentration of |
| | CSS peripheral | | receptor 1 in the peripheral |
| | (#/cell) | | compartment in the absence of |
| | | | drug. Receptor 1 bound to ligand 1 |
| | | | is included in this quantity, but |
| | | | soluble receptor 1 is not. |
| C_{SS, R2, peripheral} | Receptor 2 | 10000 | The steady-state concentration of |
| | CSS peripheral | | receptor 2 in the peripheral |
| | (#/cell) | | compartment in the absence of |
| | | | drug. Receptor 2 bound to ligand 2 |
| | | | is included in this quantity, but |
| | | | soluble receptor 2 is not. |
| C_{SS, R1, disease} | Receptor 1 | 10000 | The steady-state concentration of |
| | CSS disease | | receptor 1 in the disease |
| | (#/cell) | | compartment in the absence of |
| | | | drug. Receptor 1 bound to ligand 1 |
| | | | is included in this quantity, but |
| | | | soluble receptor 1 is not. |
| C_{SS, R2, disease} | Receptor 2 | 10000 | The steady-state concentration of |
| | CSS disease | | receptor 2 in the disease |
| | (#/cell) | | compartment in the absence of |
| | | | drug. Receptor 2 bound to ligand 2 |
| | | | is included in this quantity, but |
| | | | soluble receptor 2 is not. |
| C_{SS, R1, tox} | Receptor 1 | 10000 | The steady-state concentration of |
| | CSS tox | | receptor 1 in the tox compartment |
| | (#/cell) | | in the absence of drug. Receptor 1 |
| | | | bound to ligand 1 is included in this |
| | | | quantity, but soluble receptor 1 is |
| | | | not. |
| C_{SS, R2, tox} | Receptor 2 | 10000 | The steady-state concentration of |
| | CSS tox | | receptor 2 in the tox compartment |
| | (#/cell) | | in the absence of drug. Receptor 2 |
| | | | bound to ligand 2 is included in this |
| | | | quantity, but soluble receptor 2 is |
| | | | not. |
| C_{SS, sR1, central} | Soluble receptor | 0 | The steady-state concentration of |
| | 1 CSS central | | soluble receptor 1 in the central |
| | (nM) | | compartment in the absence of |
| | | | drug. |
| C_{SS, sR2, central} | Soluble receptor | 0 | The steady-state concentration of |
| | 2 CSS central | | soluble receptor 2 in the central |
| | (nM) | | compartment in the absence of |
| | | | drug. |
| C_{SS, sR1, peripheral} | Soluble receptor | 0 | The steady-state concentration of |
| | 1 CSS peripheral | | soluble receptor 1 in the peripheral |
| | (nM) | | compartment in the absence of |
| | | | drug. |
| C_{SS, sR2, peripheral} | Soluble receptor | 0 | The steady-state concentration of |
| | 2 CSS peripheral | | soluble receptor 2 in the peripheral |
| | (nM) | | compartment in the absence of |
| | | | drug. |
| C_{SS, sR1, disease} | Soluble receptor | 0 | The steady-state concentration of |
| | 1 CSS disease | | soluble receptor 1 in the disease |
| | (nM) | | compartment in the absence of |
| | | | drug. |
| C_{SS, sR2, disease} | Soluble receptor | 0 | The steady-state concentration of |
| | 2 CSS disease | | soluble receptor 2 in the disease |
| | (nM) | | compartment in the absence of |
| | | | drug. |
| C_{SS, sR1, tox} | Soluble receptor | 0 | The steady-state concentration of |
| | 1 CSS tox | | soluble receptor 1 in the tox |
| | (nM) | | compartment in the absence of |
| | | | drug. |
| C_{SS, sR2, tox} | Soluble receptor | 0 | The steady-state concentration of |
| | 2 CSS tox | | soluble receptor 2 in the tox |
| | (nM) | | compartment in the absence of |
| | | | drug. |
| T_{dist, L1, peripheral} | Ligand 1 Tdist | 30 | The distribution rate of ligand 1 |
| | peripheral | | from the central compartment to the |
| | (hr) | | peripheral compartment. |
| T_{dist, L2, peripheral} | Ligand 2 Tdist | 30 | The distribution rate of ligand 2 |
| | peripheral | | from the central compartment to the |
| | (hr) | | peripheral compartment. |
| T_{dist, L1, disease} | Ligand 1 Tdist | 30 | The distribution rate of ligand 1 |
| | disease | | from the central compartment to the |
| | (hr) | | disease compartment. |
| T_{dist, L2, disease} | Ligand 2 Tdist | 30 | The distribution rate of ligand 2 |
| | disease | | from the central compartment to the |
| | (hr) | | disease compartment. |
| T_{dist, L1, tox} | Ligand 1 Tdist | 30 | The distribution rate of ligand 1 |
| | tox | | from the central compartment to the |
| | (hr) | | tox compartment. |
| T_{dist, L2, tox} | Ligand 2 Tdist | 30 | The distribution rate of ligand 2 |
| | tox | | from the central compartment to the |
| | (hr) | | tox compartment. |
| T_{dist, sR1, peripheral} | Soluble receptor | 30 | The distribution rate of soluble |
| | 1 Tdist | | receptor 1 from the central |
| | peripheral | | compartment to the peripheral |
| | (hr) | | compartment. |
| T_{dist, sR2, peripheral} | Soluble receptor | 30 | The distribution rate of soluble |
| | 2 Tdist peripheral | | receptor 2 from the central |
| | (hr) | | compartment to the peripheral |
| | | | compartment. |
| T_{dist, sR1, disease} | Soluble receptor | 30 | The distribution rate of soluble |
| | 1 Tdist disease | | receptor 1 from the central |
| | (hr) | | compartment to the disease |
| | | | compartment. |
| T_{dist, sR2, disease} | Soluble receptor | 30 | The distribution rate of soluble |
| | 2 Tdist disease | | receptor 2 from the central |
| | (hr) | | compartment to the disease |
| | | | compartment. |
| T_{dist, sR1, tox} | Soluble receptor | 30 | The distribution rate of soluble |
| | 1 Tdist tox | | receptor 1 from the central |
| | (hr) | | compartment to the tox |
| | | | compartment. |
| T_{dist, sR2, tox} | Soluble receptor | 30 | The distribution rate of soluble |
| | 2 Tdist tox | | receptor 2 from the central |
| | (hr) | | compartment to the tox |
| | | | compartment. |
| Diam_{cell} | Cell diameter (um) | 10 | Diameter of receptor-bearing cells. |
| Densityy_{cells, central} | Cell density | 1000000 | Density of receptor-bearing cells in |
| | central (#/mL) | | the central compartment. |
| Density_{cells, peripheral} | Cell density | 1000000 | Density of receptor-bearing cells in |
| | peripheral (#/mL) | | the peripheral compartment. |
| Density_{cells, disease} | Cell density | 1000000 | Density of receptor-bearing cells in |
| | disease (#/mL) | | the disease compartment. |
| Density_{cells, tox} | Cell density | 1000000 | Density of receptor-bearing cells in |
| | tox (#/mL) | | the tox compartment. |
| Scale_{t1/2, R2, D:R2, central} | Drug: Receptor | 1 | A multiplicative factor for receptor |
| | 2 T ½ scale | | 2 turnover half-life when drug is |
| | central | | bound to it in the central |
| | | | compartment. Can be used to model |
| | | | effects the drug has on the |
| | | | internalization rate of this target. |
| Scale_{t1/2, R2, D:R2, peripheral} | Drug: Receptor | 1 | A multiplicative factor for receptor |
| | 2 T ½ scale | | 2 turnover half-life when drug is |
| | peripheral | | bound to it in the peripheral |
| | | | compartment. Can be used to model |
| | | | effects the drug has on the |
| | | | internalization rate of this target. |
| Scale_{t1/2, R2, D:R2, disease} | Drug: Receptor | 1 | A multiplicative factor for receptor |
| | 2 T ½ scale | | 2 turnover half-life when drug is |
| | disease | | bound to it in the disease |
| | | | compartment. Can be used to model |
| | | | effects the drug has on the |
| | | | internalization rate of this target. |
| Scale_{t1/2, R2, D:R2, tox} | Drug: Receptor | 1 | A multiplicative factor for receptor |
| | 2 T ½ scale | | 2 turnover half-life when drug is |
| | tox | | bound to it in the tox compartment. |
| | | | Can be used to model effects the |
| | | | drug has on the internalization rate |
| | | | of this target. |
| Scale_{KD, L1, central} | Drug: Target | 1 | A multiplicative factor for the |
| | 1 Kd scale | | equilibrium dissociation constant |
| | central | | between drug and ligand 1 in the |
| | | | central compartment. Can be used |
| | | | to model drugs whose target affinity |
| | | | is affected by the compartment. |
| Scale_{KD, R2, central} | Drug: Target | 1 | A multiplicative factor for the |
| | 2 Kd scale | | equilibrium dissociation constant |
| | central | | between drug and receptor 2 in the |
| | | | central compartment. Can be used |
| | | | to model drugs whose target affinity |
| | | | is affected by the compartment. |
| Scale_{KD, L1, peripheral} | Drug: Target | 1 | A multiplicative factor for the |
| | 1 Kd scale | | equilibrium dissociation constant |
| | peripheral | | between drug and ligand 1 in the |
| | | | peripheral compartment. Can be |
| | | | used to model drugs whose target |
| | | | affinity is affected by the |
| | | | compartment. |
| Scale_{KD, R2, peripheral} | Drug: Target | 1 | A multiplicative factor for the |
| | 2 Kd scale | | equilibrium dissociation constant |
| | peripheral | | between drug and receptor 2 in the |
| | | | peripheral compartment. Can be |
| | | | used to model drugs whose target |
| | | | affinity is affected by the |
| | | | compartment. |
| Scale_{KD, L1, disease} | Drug: Target | 1 | A multiplicative factor for the |
| | 1 Kd scale | | equilibrium dissociation constant |
| | disease | | between drug and ligand 1 in the |
| | | | disease compartment. Can be used |
| | | | to model drugs whose target affinity |
| | | | is affected by the compartment. |
| Scale_{KD, R2, disease} | Drug: Target | 1 | A multiplicative factor for the |
| | 2 Kd scale | | equilibrium dissociation constant |
| | disease | | between drug and receptor 2 in the |
| | | | disease compartment. Can be used |
| | | | to model drugs whose target affinity |
| | | | is affected by the compartment. |
| Scale_{KD, L1, tox} | Drug: Target | 1 | A multiplicative factor for the |
| | 1 Kd scale | | equilibrium dissociation constant |
| | tox | | between drug and ligand 1 in the |
| | | | tox compartment. Can be used to |
| | | | model drugs whose target affinity is |
| | | | affected by the compartment. |
| Scale_{KD, R2, tox} | Drug: Target | 1 | A multiplicative factor for the |
| | 2 Kd scale | | equilibrium dissociation constant |
| | tox | | between drug and receptor 2 in the |
| | | | tox compartment. Can be used to |
| | | | model drugs whose target affinity is |
| | | | affected by the compartment. |
| Scale_{t1/2, central} | Soluble Drug | 1 | A multiplicative factor for the drug |
| | T ½ scale | | elimination half-life in the central |
| | central | | compartment. Can be used to model |
| | | | drugs which have a different half- |
| | | | life in some compartments. |
| Scale_{t1/2, peripheral} | Soluble Drug | 1 | A multiplicative factor for the drug |
| | T ½ scale | | elimination half-life in the |
| | peripheral | | peripheral compartment. Can be |
| | | | used to model drugs which have a |
| | | | different half-life in some |
| | | | compartments. |
| Scale_{t1/2, disease} | Soluble Drug | 1 | A multiplicative factor for the drug |
| | T ½ scale | | elimination half-life in the disease |
| | disease | | compartment. Can be used to model |
| | | | drugs which have a different half- |
| | | | life in some compartments. |
| Scale_{t1/2, tox} | Soluble Drug | 1 | A multiplicative factor for the drug |
| | T ½ scale | | elimination half-life in the tox |
| | tox | | compartment. Can be used to model |
| | | | drugs which have a different half- |
| | | | life in some compartments. |

TABLE 14
Example target criteria (e.g., for the therapeutic drug
candidate and the target for the therapeutic purpose)
of the bispecific anti-ligand × anti-receptor with
avidity (4-compartment) example model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free |
| Engagement | | | target due to drug binding. |
| | | | Measured as a percent of untreated. |
| Activation | 90 | percent | Percent of receptor occupied |
| | | | by ligand or drug. |
| | | | Measured as a percent of total. |
| | | | This is typically used |
| | | | for agonists. |

TABLE 15
Example output plots of the bispecific anti-ligand × anti-receptor
with avidity (4-compartment) example model in the EFA system.

| Output Plot | Description |
|---|---|
| Criterion | The value of the selected criterion |
| vs scan | for each value of the 1D scan |
| parameter | parameter simulated. The desired |
| | target value is plotted as a |
| | horizontal dashed line. |
| Criterion | The value of the selected criterion |
| heatmap | for pair of 2D scan |
| | parameters. The range of the |
| | heatmap color bar is fixed for each |
| | criterion. |
| Criterion | A time course plot of the selected |
| time course | criterion's time value. |
| Free drug | A time course plot of the |
| | concentration of drug not bound to anything. |
| Total drug | A time course plot of the concentration |
| | of soluble drug. This is any form of the |
| | drug not bound to any membrane proteins. |
| | This may include drug bound to one or |
| | more soluble proteins such as |
| | ligands or soluble receptors. |

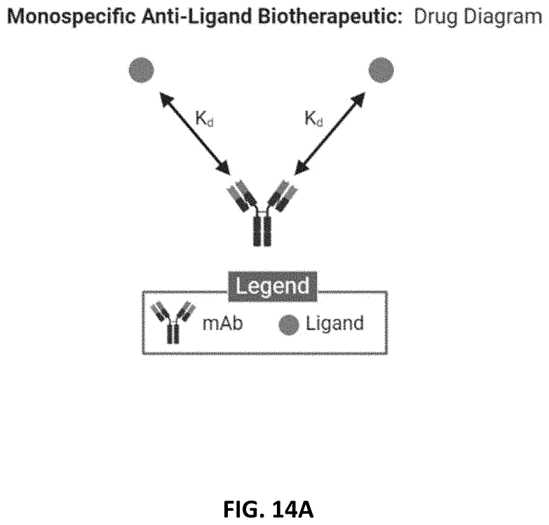

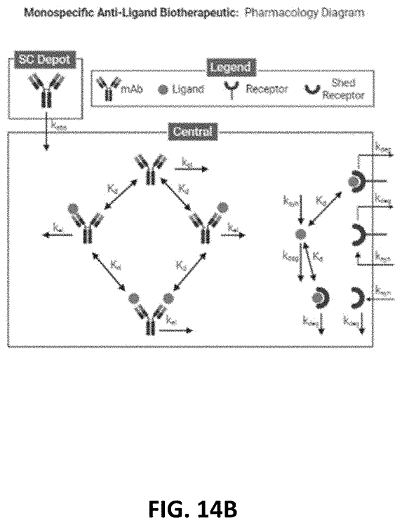

In some implementations, the example monospecific anti-ligand model can include an one-compartment model of bivalent antibody binding to soluble ligand. A biologic that binds to a soluble ligand target and prevents the ligand from binding to its cognate-receptor. The molecule can be mono- or bivalent. This is a one-compartment model and there is an option for +/− soluble receptor. FIGS. 14A-14B show schematic diagrams illustrating a biotherapeutic scenario of the example monospecific anti-ligand model, according to some embodiments. Example parameters of the example monospecific anti-ligand model in the EFA system are shown in Table 16. Example target criteria (e.g., for the therapeutic drug candidate and the target for the therapeutic purpose) of the example monospecific anti-ligand model in the EFA system are shown in Table 17. Example output plots of the example monospecific anti-ligand model in the EFA system are shown in Table 18.

TABLE 16
Example parameters of the example monospecific
anti-ligand model in the EFA system.

| Symbol | Name | Default | Definition |
|---|---|---|---|
| τ | Interval | 14 | The interval between doses of the |
| | | | drug. |
| D | Dose (mg) | 100 | The amount of each dose. |
| K_{D, L} | Drug: Target | 0.1 | The affinity of the drug for ligand |
| | KD (nM) | | as characterized by the equilibrium |
| | | | dissociation constant. This is the |
| | | | monovalent affinity (i.e. the affinity |
| | | | of single binding). |
| N_{doses} | Number of | 7 | The number of doses to be given. |
| | doses | | |
| MW | Molecular | 150000 | The molecular weight of the drug. |
| | Weight (Da) | | This is used to convert the dose |
| | | | which is expressed in mass units to |
| | | | a molar dose. |
| t_{1/2} | Biologic first | 28 | The first-order elimination rate of |
| | order T ½ | | the drug. |
| | (days) | | |
| t_{1/2, a} | SC absorption | 2.5 | The first-order absorption rate of |
| | T ½ (days) | | the drug from the subcutaneous |
| | | | compartment into the central |
| | | | compartment. |
| BW | Body | 70 | The body weight of the subject. |
| | Weight (kg) | | This is used to convert the dose |
| | | | when expressed in units of mg/kg. |
| V | Volume (L) | 5 | The volume of the central |
| | | | compartment which is typically the |
| | | | plasma of the peripheral blood. |
| Valency | Effective | 2 | The number of ligand molecules |
| | Valency | | that a molecule of drug can bind. |
| | | | This may be 1 or 2. |
| t_{1/2, L} | Ligand | 0.5 | The first-order elimination rate of |
| | T ½ (hr) | | ligand from the central |
| | | | compartment. |
| t_{1/2, R} | Receptor | 1 | The first-order turnover rate of |
| | T ½ (hr) | | receptor in all compartments. |
| t_{1/2, sR} | Soluble receptor | 0.5 | The first-order elimination rate of |
| | T ½ (hr) | | soluble receptor from the central |
| | | | compartment. |
| K_{D, L:R} | L:R Affinity | 1 | The binding affinity of ligand for |
| | (nM) | | receptor as characterized by the |
| | | | equilibrium dissociation constant. |
| C_{SS, L} | Ligand | 0.05 | The steady-state concentration of |
| | CSS (nM) | | free ligand in the compartment in |
| | | | the absence of drug. Ligand bound |
| | | | to receptor is not included in this |
| | | | quantity. |
| C_{SS, R} | Receptor | 10000 | The steady-state concentration of |
| | CSS (#/cell) | | receptor in the compartment in the |
| | | | absence of drug. Receptor bound to |
| | | | ligand is included in this quantity, |
| | | | but soluble receptor is not. |
| C_{SS, sR} | Soluble | 0 | The steady-state concentration of |
| | receptor CSS (nM) | | soluble receptor in the compartment |
| | | | in the absence of drug. |
| Density_{cells} | Cell | 1000000 | Density of receptor-bearing cells in |
| | density (#/mL) | | the compartment. |

TABLE 17
Example target criteria (e.g., for the therapeutic drug candidate
and the target for the therapeutic purpose) of the example
monospecific anti-ligand model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free |
| Engagement | | | target due to drug binding. |
| | | | Measured as a percent of untreated. |
| Activation | 90 | percent | Percent of receptor occupied |
| | | | by ligand or drug. |
| | | | Measured as a percent of total. |
| | | | This is typically used |
| | | | for agonists. |

TABLE 18
Example output plots of the example monospecific
anti-ligand model in the EFA system.

| Output Plot | Description |
|---|---|
| Criterion | The value of the selected criterion |
| vs scan | for each value of the 1D scan |
| parameter | parameter simulated. The desired target |
| | value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is any form of the |
| | drug not bound to any membrane proteins. |
| | This may include drug bound to one or |
| | more soluble proteins such as |
| | ligands or soluble receptors. |

In some implementations, the example monospecific anti-ligand (4-compartment) model can include a four-compartment model of bivalent antibody binding to soluble ligand. A biologic that binds to a soluble ligand target and prevents the ligand from binding to its cognate-receptor. The molecule can be mono- or bivalent. This is a four-compartment model with a central, peripheral, disease and tox compartments. There is an option for +/− soluble receptor. FIG. 14A shows a schematic diagram illustrating a biotherapeutic scenario of the example monospecific anti-ligand (4-compartment) model, according to some embodiments. Example parameters of the example monospecific anti-ligand (4-compartment) model in the EFA system are shown in Table 19. Example target criteria (e.g., for the therapeutic drug candidate and the target for the therapeutic purpose) of the example monospecific anti-ligand (4-compartment) model in the EFA system are shown in Table 20. Example output plots of the example monospecific anti-ligand (4-compartment) model in the EFA system are shown in Table 21.

TABLE 19
Example parameters of the example monospecific anti-
ligand (4-compartment) model in the EFA system.

| Symbol | Name | Default | Definition |
|---|---|---|---|
| τ | Interval | 14 | The interval between doses of the drug. |
| D | Dose (mg) | 100 | The amount of each dose. |
| K_{D, L} | Drug: | 0.1 | The affinity of the drug for ligand as characterized by |
| | Target | | the equilibrium dissociation constant. This is the |
| | KD (nM) | | monovalent affinity (i.e. the affinity of single |
| | | | binding). |
| N_{doses} | Number of | 7 | The number of doses to be given. |
| | doses | | |
| MW | Molecular | 150000 | The molecular weight of the drug. This is used to |
| | Weight | | convert the dose which is expressed in mass units to |
| | (Da) | | a molar dose. |
| t_{1/2} | Biologic | 28 | The first-order elimination rate of the drug. |
| | first | | |
| | order | | |
| | T ½ | | |
| | (days) | | |
| t_{1/2, a} | SC | 2.5 | The first-order absorption rate of the drug from the |
| | absorption | | subcutaneous compartment into the central |
| | T ½ | | compartment. |
| | (days) | | |
| BW | Body | 70 | The body weight of the subject. This is used to |
| | Weight | | convert the dose when expressed in units of mg/kg. |
| | (kg) | | |
| V | Volume | 2.5 | The volume of the central compartment which is |
| | central | | typically the plasma of the peripheral blood. |
| | (L) | | |
| Vₚₑᵣᵢₚₕₑᵣₐₗ | Volume | 12.8 | The volume of the non-blood fluids that the antibody |
| | peripheral | | can distribute to. |
| | (L) | | |
| V_{disease} | Volume | 0.1 | The volume of the disease compartment interstitial |
| | disease | | fluid. |
| | (L) | | |
| Vₜₒₓ | Volume | 0.1 | The volume of the tox compartment interstitial fluid. |
| | tox | | |
| | (L) | | |
| T_{dist, peripheral} | Drug | 30 | The distribution rate of drug from the central |
| | Tdist | | compartment to the peripheral compartment. |
| | peripheral | | |
| | (hr) | | |
| T_{dist, disease} | Drug | 30 | The distribution rate of drug from the central |
| | Tdist | | compartment to the disease compartment. |
| | disease | | |
| | (hr) | | |
| T_{dist, tox} | Drug | 30 | The distribution rate of drug from the central |
| | Tdist | | compartment to the tox compartment. |
| | tox | | |
| | (hr) | | |
| P_{dist, peripheral} | Drug | 0.190625 | The steady-state ratio of soluble drug in the |
| | partition | | peripheral compartment to soluble drug in the central |
| | coefficient | | compartment. |
| | peripheral | | |
| P_{dist, disease} | Drug | 0.3 | The steady-state ratio of soluble drug in the disease |
| | partition | | compartment to soluble drug in the central |
| | coefficient | | compartment. |
| | disease | | |
| P_{dist, tox} | Drug | 0.3 | The steady-state ratio of soluble drug in the tox |
| | partition | | compartment to soluble drug in the central |
| | coefficient | | compartment. |
| | tox | | |
| Valency | Effective | 2 | The number of ligand molecules that a molecule of |
| | Valency | | drug can bind. This may be 1 or 2. |
| t_{1/2, L} | Ligand | 0.5 | The first-order elimination rate of ligand from the |
| | T ½ | | central compartment. |
| | (hr) | | |
| t_{1/2, R} | Receptor | 1 | The first-order turnover rate of receptor in all |
| | T ½ | | compartments. |
| | (hr) | | |
| t_{1/2, sR} | Soluble | 0.5 | The first-order elimination rate of soluble receptor |
| | receptor | | from the central compartment. |
| | T ½ | | |
| | (hr) | | |
| K_{D, L:R} | L:R | 1 | The binding affinity of ligand for receptor as |
| | Affinity | | characterized by the equilibrium dissociation |
| | (nM) | | constant. |
| C_{SS, L, central} | Ligand | 0.05 | The steady-state concentration of free ligand in the |
| | CSS | | central compartment in the absence of drug. Ligand |
| | central | | bound to receptor is not included in this quantity. |
| | (nM) | | |
| C_{SS, L, peripheral} | Ligand | 0.05 | The steady-state concentration of free ligand in the |
| | CSS | | peripheral compartment in the absence of drug. |
| | peripheral | | Ligand bound to receptor is not included in this |
| | (nM) | | quantity. |
| C_{SS, L, disease} | Ligand | 0.05 | The steady-state concentration of free ligand in the |
| | CSS | | disease compartment in the absence of drug. Ligand |
| | disease | | bound to receptor is not included in this quantity. |
| | (nM) | | |
| C_{SS, L, tox} | Ligand | 0.05 | The steady-state concentration of free ligand in the |
| | CSS | | tox compartment in the absence of drug. Ligand |
| | tox | | bound to receptor is not included in this quantity. |
| | (nM) | | |
| C_{SS, R, central} | Receptor | 10000 | The steady-state concentration of receptor in the |
| | CSS | | central compartment in the absence of drug. Receptor |
| | central | | bound to ligand is included in this quantity, but |
| | (#/cell) | | soluble receptor is not. |
| C_{SS, R, peripheral} | Receptor | 10000 | The steady-state concentration of receptor in the |
| | CSS | | peripheral compartment in the absence of drug. |
| | peripheral | | Receptor bound to ligand is included in this quantity, |
| | (#/cell) | | but soluble receptor is not. |
| C_{SS, R, disease} | Receptor | 10000 | The steady-state concentration of receptor in the |
| | CSS | | disease compartment in the absence of drug. |
| | disease | | Receptor bound to ligand is included in this quantity, |
| | (#/cell) | | but soluble receptor is not. |
| C_{SS, R, tox} | Receptor | 10000 | The steady-state concentration of receptor in the tox |
| | CSS | | compartment in the absence of drug. Receptor bound |
| | tox | | to ligand is included in this quantity, but soluble |
| | (#/cell) | | receptor is not. |
| C_{SS, sR, central} | Soluble | 0 | The steady-state concentration of soluble receptor in |
| | receptor | | the central compartment in the absence of drug. |
| | CSS | | |
| | central | | |
| | (nM) | | |
| C_{SS, sR, peripheral} | Soluble | 0 | The steady-state concentration of soluble receptor in |
| | receptor | | the peripheral compartment in the absence of drug. |
| | CSS | | |
| | peripheral | | |
| | (nM) | | |
| C_{SS, sR, disease} | Soluble | 0 | The steady-state concentration of soluble receptor in |
| | receptor | | the disease compartment in the absence of drug. |
| | CSS | | |
| | disease | | |
| | (nM) | | |
| C_{SS, sR, tox} | Soluble | 0 | The steady-state concentration of soluble receptor in |
| | receptor | | the tox compartment in the absence of drug. |
| | CSS | | |
| | tox | | |
| | (nM) | | |
| T_{dist, L, peripheral} | Ligand | 30 | The distribution rate of ligand from the central |
| | Tdist | | compartment to the peripheral compartment. |
| | peripheral | | |
| | (hr) | | |
| T_{dist, L, disease} | Ligand | 30 | The distribution rate of ligand from the central |
| | Tdist | | compartment to the disease compartment. |
| | disease | | |
| | (hr) | | |
| T_{dist, L, tox} | Ligand | 30 | The distribution rate of ligand from the central |
| | Tdist | | compartment to the tox compartment. |
| | tox | | |
| | (hr) | | |
| T_{dist, sR, peripheral} | Soluble | 30 | The distribution rate of soluble receptor from the |
| | receptor | | central compartment to the peripheral compartment. |
| | Tdist | | |
| | peripheral | | |
| | (hr) | | |
| T_{dist, sR, disease} | Soluble | 30 | The distribution rate of soluble receptor from the |
| | receptor | | central compartment to the disease compartment. |
| | Tdist | | |
| | disease | | |
| | (hr) | | |
| T_{dist, sR, tox} | Soluble | 30 | The distribution rate of soluble receptor from the |
| | receptor | | central compartment to the tox compartment. |
| | Tdist | | |
| | tox | | |
| | (hr) | | |
| Density_{cells, central} | Cell | 1000000 | Density of receptor-bearing cells in the central |
| | density | | compartment. |
| | central | | |
| | (#/mL) | | |
| Density_{cells, peripheral} | Cell | 1000000 | Density of receptor-bearing cells in the peripheral |
| | density | | compartment. |
| | peripheral | | |
| | (#/mL) | | |
| Density_{cells, disease} | Cell | 1000000 | Density of receptor-bearing cells in the disease |
| | density | | compartment. |
| | disease | | |
| | (#/mL) | | |
| Density_{cells, tox} | Cell | 1000000 | Density of receptor-bearing cells in the tox |
| | density | | compartment. |
| | tox | | |
| | (#/mL) | | |
| Scale_{KD, L, central} | Drug: | 1 | A multiplicative factor for the equilibrium |
| | Target | | dissociation constant between drug and ligand in the |
| | Kd | | central compartment. Can be used to model drugs |
| | scale | | whose target affinity is affected by the compartment. |
| | central | | |
| Scale_{KD, L, peripheral} | Drug: | 1 | A multiplicative factor for the equilibrium |
| | Target | | dissociation constant between drug and ligand in the |
| | Kd | | peripheral compartment. Can be used to model drugs |
| | scale | | whose target affinity is affected by the compartment. |
| | peripheral | | |
| Scale_{KD, L, disease} | Drug: | 1 | A multiplicative factor for the equilibrium |
| | Target | | dissociation constant between drug and ligand in the |
| | Kd | | disease compartment. Can be used to model drugs |
| | scale | | whose target affinity is affected by the compartment. |
| | disease | | |
| Scale_{KD, L, tox} | Drug: | 1 | A multiplicative factor for the equilibrium |
| | Target | | dissociation constant between drug and ligand in the |
| | Kd | | tox compartment. Can be used to model drugs whose |
| | scale | | target affinity is affected by the compartment. |
| | tox | | |
| Scale_{t1/2, central} | Soluble | 1 | A multiplicative factor for the drug elimination half- |
| | Drug | | life in the central compartment. Can be used to model |
| | T ½ | | drugs which have a different half-life in some |
| | scale | | compartments. |
| | central | | |
| Scale_{t1/2, peripheral} | Soluble | 1 | A multiplicative factor for the drug elimination half- |
| | Drug | | life in the peripheral compartment. Can be used to |
| | T ½ | | model drugs which have a different half-life in some |
| | scale | | compartments. |
| | peripheral | | |
| Scale_{t1/2, disease} | Soluble | 1 | A multiplicative factor for the drug elimination half- |
| | Drug | | life in the disease compartment. Can be used to |
| | T ½ | | model drugs which have a different half-life in some |
| | scale | | compartments. |
| | disease | | |
| Scale_{t1/2, tox} | Soluble | 1 | A multiplicative factor for the drug elimination half- |
| | Drug | | life in the tox compartment. Can be used to model |
| | T ½ | | drugs which have a different half-life in some |
| | scale | | compartments. |
| | tox | | |

TABLE 20
Example target criteria (e.g., for the therapeutic drug candidate
and the target for the therapeutic purpose) of the example monospecific
anti-ligand (4-compartment) model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free |
| Engagement | | | target due to drug binding. |
| | | | Measured as a percent of untreated. |
| Activation | 90 | percent | Percent of receptor occupied |
| | | | by ligand or drug. |
| | | | Measured as a percent of |
| | | | total. This is typically used |
| | | | for agonists. |

TABLE 21
Example output plots of the example monospecific anti-
ligand (4-compartment) model in the EFA system.

| Output Plot | Description |
|---|---|
| Criterion | The value of the selected criterion |
| vs scan | for each value of the 1D scan |
| parameter | parameter simulated. The desired |
| | target value is plotted as a |
| | horizontal dashed line. |
| Criterion | The value of the selected criterion |
| heatmap | for pair of 2D scan |
| | parameters. The range of the |
| | heatmap color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is any form of the |
| | drug not bound to any membrane proteins. |
| | This may include drug bound to one or |
| | more soluble proteins such as |
| | ligands or soluble receptors. |

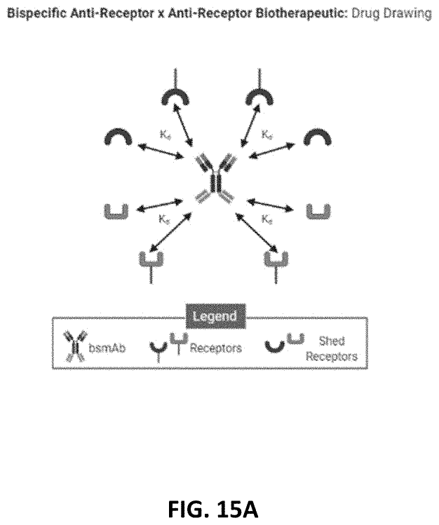

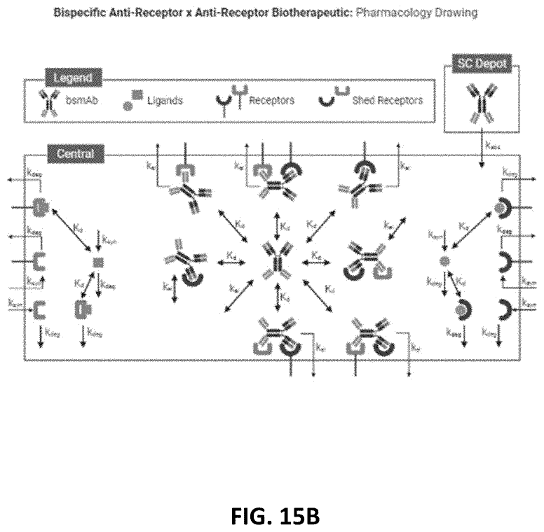

In some implementations, the example bispecific anti-receptor x anti-receptor model can include an one-compartment model of bivalent antibody binding to two different membrane receptors. A bispecific biotherapeutic that binds to two target cell surface receptors on a single cell type. For each receptor, the drug either (1) acts as a competitive inhibitor by blocking the cognate-ligand from binding to its receptor or (2) acts as a receptor agonist. The molecule can be mono- or bivalent for each target. This is a one-compartment model and there is an option for +/− soluble receptor. FIGS. 15A-15B show schematic diagrams illustrating a biotherapeutic scenario of the example bispecific anti-receptor x anti-receptor model, according to some embodiments. Example parameters of the example bispecific anti-receptor x anti-receptor model in the EFA system are shown in Table 22. Example target criteria (e.g., for the therapeutic drug candidate and the target for the therapeutic purpose) of the example bispecific anti-receptor x anti-receptor model in the EFA system are shown in Table 23. Example output plots of the example bispecific anti-receptor x anti-receptor model in the EFA system are shown in Table 24.

TABLE 22
Example parameters of the example bispecific anti-
receptor × anti-receptor model in the EFA system.

| Symbol | Name | Default | Definition |
|---|---|---|---|
| τ | Interval | 14 | The interval between doses of the drug. |
| D | Dose | 100 | The amount of each dose. |
| | (mg) | | |
| K_{D, R1} | Drug: | 0.1 | The affinity of the drug for receptor 1 as characterized |
| | Target 1 | | by the equilibrium dissociation constant. This is the |
| | KD | | monovalent affinity (i.e. the affinity of single binding). |
| | (nM) | | |
| K_{D, R2} | Drug: | 0.1 | The affinity of the drug for receptor 2 as characterized |
| | Target 2 | | by the equilibrium dissociation constant. This is the |
| | KD | | monovalent affinity (i.e. the affinity of single binding). |
| | (nM) | | |
| N_{doses} | Number of | 7 | The number of doses to be given. |
| | doses | | |
| MW | Molecular | 150000 | The molecular weight of the drug. This is used to |
| | Weight | | convert the dose which is expressed in mass units to a |
| | (Da) | | molar dose. |
| t_{1/2} | Biologic | 28 | The first-order elimination rate of the drug. |
| | first | | |
| | order | | |
| | T ½ | | |
| | (days) | | |
| t_{1/2, a} | SC | 2.5 | The first-order absorption rate of the drug from the |
| | absorption | | subcutaneous compartment into the central |
| | T ½ | | compartment. |
| | (days) | | |
| BW | Body | 70 | The body weight of the subject. This is used to convert |
| | Weight | | the dose when expressed in units of mg/kg. |
| | (kg) | | |
| V | Volume | 5 | The volume of the central compartment which is |
| | (L) | | typically the plasma of the peripheral blood. |
| Valency₁ | Effective | 2 | The number of receptor 1 molecules that a molecule of |
| | Valency 1 | | drug can bind. This may be 1 or 2. |
| Valency₂ | Effective | 2 | The number of receptor 2 molecules that a molecule of |
| | Valency 2 | | drug can bind. This may be 1 or 2. |
| t_{1/2, L1} | Ligand 1 | 0.5 | The first-order elimination rate of ligand 1 from the |
| | T ½ | | central compartment. |
| | (hr) | | |
| t_{1/2, L2} | Ligand 2 | 0.5 | The first-order elimination rate of ligand 2 from the |
| | T ½ | | central compartment. |
| | (hr) | | |
| t_{1/2, R1} | Receptor 1 | 1 | The first-order turnover rate of receptor 1 in all |
| | T ½ | | compartments. |
| | (hr) | | |
| t_{1/2, R2} | Receptor 2 | 1 | The first-order turnover rate of receptor 2 in all |
| | T ½ | | compartments. |
| | (hr) | | |
| t_{1/2, sR1} | Soluble | 0.5 | The first-order elimination rate of soluble receptor 1 |
| | receptor 1 | | from the central compartment. |
| | T ½ | | |
| | (hr) | | |
| t_{1/2, sR2} | Soluble | 0.5 | The first-order elimination rate of soluble receptor 2 |
| | receptor 2 | | from the central compartment. |
| | T ½ | | |
| | (hr) | | |
| K_{D, L1:R1} | L1:R1 | 1 | The binding affinity of ligand 1 for receptor 1 as |
| | Affinity | | characterized by the equilibrium dissociation constant. |
| | (nM) | | |
| K_{D, L2:R2} | L2:R2 | 1 | The binding affinity of ligand 2 for receptor 2 as |
| | Affinity | | characterized by the equilibrium dissociation constant. |
| | (nM) | | |
| C_{SS, L1} | Ligand 1 | 0.05 | The steady-state concentration of free ligand 1 in the |
| | CSS | | compartment in the absence of drug. Ligand 1 bound |
| | (nM) | | to receptor 1 is not included in this quantity. |
| C_{SS, L2} | Ligand 2 | 0.05 | The steady-state concentration of free ligand 2 in the |
| | CSS | | compartment in the absence of drug. Ligand 2 bound |
| | (nM) | | to receptor 2 is not included in this quantity. |
| C_{SS, R1} | Receptor 1 | 10000 | The steady-state concentration of receptor 1 in the |
| | CSS | | compartment in the absence of drug. Receptor 1 bound |
| | (#/cell) | | to ligand 1 is included in this quantity, but soluble |
| | | | receptor 1 is not. |
| C_{SS, R2} | Receptor 2 | 10000 | The steady-state concentration of receptor 2 in the |
| | CSS | | compartment in the absence of drug. Receptor 2 bound |
| | (#/cell) | | to ligand 2 is included in this quantity, but soluble |
| | | | receptor 2 is not. |
| C_{SS, sR1} | Soluble | 0 | The steady-state concentration of soluble receptor 1 in |
| | receptor 1 | | the compartment in the absence of drug. |
| | CSS | | |
| | (nM) | | |
| C_{SS, sR2} | Soluble | 0 | The steady-state concentration of soluble receptor 2 in |
| | receptor 2 | | the compartment in the absence of drug. |
| | CSS | | |
| | (nM) | | |
| Density_{cells} | Cell | 1000000 | Density of receptor-bearing cells in the compartment. |
| | density | | |
| | (#/mL) | | |
| Scale_{t1/2, R1, D:R1} | Drug: | 1 | A multiplicative factor for receptor 1 turnover half-life |
| | Receptor 1 | | when drug is bound to it in the compartment. Can be |
| | T ½ | | used to model effects the drug has on the |
| | scale | | internalization rate of this target. |
| Scale_{t1/2, R2, D:R2} | Drug: | 1 | A multiplicative factor for receptor 2 turnover half-life |
| | Receptor 2 | | when drug is bound to it in the compartment. Can be |
| | T ½ | | used to model effects the drug has on the |
| | scale | | internalization rate of this target. |
| Scale_{KD, R1} | Drug: | 1 | A multiplicative factor for the equilibrium dissociation |
| | Target 1 | | constant between drug and receptor 1 in the |
| | Kd | | compartment. Can be used to model drugs whose |
| | scale | | target affinity is affected by the compartment. |
| Scale_{KD, R2} | Drug: | 1 | A multiplicative factor for the equilibrium dissociation |
| | Target 2 | | constant between drug and receptor 2 in the |
| | Kd | | compartment. Can be used to model drugs whose |
| | scale | | target affinity is affected by the compartment. |

TABLE 23
Example target criteria (e.g., for the therapeutic drug candidate
and the target for the therapeutic purpose) of the example bispecific
anti-receptor × anti-receptor model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free |
| Engagement | | | target due to drug binding. |
| | | | Measured as a percent of untreated. |
| Activation | 90 | percent | Percent of receptor occupied |
| | | | by ligand or drug. |
| | | | Measured as a percent of total. |
| | | | This is typically used |
| | | | for agonists. |

TABLE 24
Example output plots of the example bispecific anti-
receptor × anti-receptor model in the EFA system.

| Output Plot | Description |
|---|---|
| Criterion | The value of the selected criterion |
| vs scan | for each value of the 1D scan |
| parameter | parameter simulated. The desired |
| | target value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is any form of the |
| | drug not bound to any membrane proteins. |
| | This may include drug bound to one or more |
| | soluble proteins such as |
| | ligands or soluble receptors. |

In some implementations, the example bispecific anti-receptor x anti-receptor (4-compartment) model can include a four-compartment model of bivalent antibody binding to two different membrane receptors. A bispecific biotherapeutic that binds to two target cell surface receptors on a single cell type. For each receptor the drug either (1) acts as a competitive inhibitor by blocking the cognate-ligand from binding to its receptor or (2) acts as a receptor agonist. The molecule can be mono- or bivalent for each target. This is a four-compartment model with a central, peripheral, disease and tox compartments. There is an option for +/− soluble receptor for each receptor. FIG. 15A shows a schematic diagram illustrating a biotherapeutic scenario of the example bispecific anti-receptor x anti-receptor (4-compartment) model, according to some embodiments. Example parameters of the example bispecific anti-receptor x anti-receptor (4-compartment) model in the EFA system are shown in Table 25. Example target criteria (e.g., for the therapeutic drug candidate and the target for the therapeutic purpose) of the example bispecific anti-receptor x anti-receptor (4-compartment) model in the EFA system are shown in Table 26. Example output plots of the example bispecific anti-receptor x anti-receptor (4-compartment) model in the EFA system are shown in Table 27.

TABLE 25
Example parameters of the example bispecific anti-receptor ×
anti-receptor (4-compartment) model in the EFA system.

| Symbol | Name | Default | Definition |
|---|---|---|---|
| τ | Interval | 14 | The interval between doses of the drug. |
| D | Dose (mg) | 100 | The amount of each dose. |
| K_{D, R1} | Drug: Target | 0.1 | The affinity of the drug for receptor 1 |
| | 1 KD (nM) | | as characterized by the equilibrium |
| | | | dissociation constant. This is the |
| | | | monovalent affinity (i.e. the affinity of |
| | | | single binding). |
| K_{D, R2} | Drug: Target | 0.1 | The affinity of the drug for receptor 2 |
| | 2 KD (nM) | | as characterized by the equilibrium |
| | | | dissociation constant. This is the |
| | | | monovalent affinity (i.e. the affinity of |
| | | | single binding). |
| N_{doses} | Number of | 7 | The number of doses to be given. |
| | doses | | |
| MW | Molecular | 150000 | The molecular weight of the drug. This |
| | Weight (Da) | | is used to convert the dose which is |
| | | | expressed in mass units to a molar |
| | | | dose. |
| t_{1/2} | Biologic first | 28 | The first-order elimination rate of the |
| | order T ½ | | drug. |
| | (days) | | |
| t_{1/2, a} | SC absorption | 2.5 | The first-order absorption rate of the |
| | T ½ | | drug from the subcutaneous |
| | (days) | | compartment into the central |
| | | | compartment. |
| BW | Body | 70 | The body weight of the subject. This is |
| | Weight (kg) | | used to convert the dose when |
| | | | expressed in units of mg/kg. |
| V | Volume | 2.5 | The volume of the central compartment |
| | central (L) | | which is typically the plasma of the |
| | | | peripheral blood. |
| Vₚₑᵣᵢₚₕₑᵣₐₗ | Volume | 12.8 | The volume of the non-blood fluids that |
| | peripheral (L) | | the antibody can distribute to. |
| V_{disease} | Volume | 0.1 | The volume of the disease |
| | disease (L) | | compartment interstitial fluid. |
| Vₜₒₓ | Volume | 0.1 | The volume of the tox compartment |
| | tox (L) | | interstitial fluid. |
| T_{dist, peripheral} | Drug Tdist | 30 | The distribution rate of drug from the |
| | peripheral | | central compartment to the peripheral |
| | (hr) | | compartment. |
| T_{dist, disease} | Drug Tdist | 30 | The distribution rate of drug from the |
| | disease (hr) | | central compartment to the disease |
| | | | compartment. |
| T_{dist, tox} | Drug Tdist | 30 | The distribution rate of drug from the |
| | tox (hr) | | central compartment to the tox |
| | | | compartment. |
| P_{dist, peripheral} | Drug partition | 0.190625 | The steady-state ratio of soluble drug in |
| | coefficient | | the peripheral compartment to soluble |
| | peripheral | | drug in the central compartment. |
| P_{dist, disease} | Drug partition | 0.3 | The steady-state ratio of soluble drug in |
| | coefficient | | the disease compartment to soluble |
| | disease | | drug in the central compartment. |
| P_{dist, tox} | Drug partition | 0.3 | The steady-state ratio of soluble drug in |
| | coefficient | | the tox compartment to soluble drug in |
| | tox | | the central compartment. |
| Valency₁ | Effective | 2 | The number of receptor 1 molecules |
| | Valency 1 | | that a molecule of drug can bind. This |
| | | | may be 1 or 2. |
| Valency₂ | Effective | 2 | The number of receptor 2 molecules |
| | Valency 2 | | that a molecule of drug can bind. This |
| | | | may be 1 or 2. |
| t_{1/2, L1} | Ligand 1 | 0.5 | The first-order elimination rate of |
| | T ½ (hr) | | ligand 1 from the central compartment. |
| t_{1/2, L2} | Ligand 2 | 0.5 | The first-order elimination rate of |
| | T ½ (hr) | | ligand 2 from the central compartment. |
| t_{1/2, R1} | Receptor 1 | 1 | The first-order turnover rate of receptor |
| | T ½ (hr) | | 1 in all compartments. |
| t_{1/2, R2} | Receptor 2 | 1 | The first-order turnover rate of receptor |
| | T ½ (hr) | | 2 in all compartments. |
| t_{1/2, sR1} | Soluble | 0.5 | The first-order elimination rate of |
| | receptor 1 | | soluble receptor 1 from the central |
| | T ½ (hr) | | compartment. |
| t_{1/2, sR2} | Soluble | 0.5 | The first-order elimination rate of |
| | receptor 2 | | soluble receptor 2 from the central |
| | T ½ (hr) | | compartment. |
| K_{D, L1:R1} | L1:R1 | 1 | The binding affinity of ligand 1 for |
| | Affinity (nM) | | receptor 1 as characterized by the |
| | | | equilibrium dissociation constant. |
| K_{D, L2:R2} | L2:R2 | 1 | The binding affinity of ligand 2 for |
| | Affinity (nM) | | receptor 2 as characterized by the |
| | | | equilibrium dissociation constant. |
| C_{SS, L1, central} | Ligand 1 | 0.05 | The steady-state concentration of free |
| | CSS central | | ligand 1 in the central compartment in |
| | (nM) | | the absence of drug. Ligand 1 bound to |
| | | | receptor 1 is not included in this |
| | | | quantity. |
| C_{SS, L2, central} | Ligand 2 | 0.05 | The steady-state concentration of free |
| | CSS central | | ligand 2 in the central compartment in |
| | (nM) | | the absence of drug. Ligand 2 bound to |
| | | | receptor 2 is not included in this |
| | | | quantity. |
| C_{SS, L1, peripheral} | Ligand 1 | 0.05 | The steady-state concentration of free |
| | CSS peripheral | | ligand 1 in the peripheral compartment |
| | (nM) | | in the absence of drug. Ligand 1 bound |
| | | | to receptor 1 is not included in this |
| | | | quantity. |
| C_{SS, L2, peripheral} | Ligand 2 | 0.05 | The steady-state concentration of free |
| | CSS peripheral | | ligand 2 in the peripheral compartment |
| | (nM) | | in the absence of drug. Ligand 2 bound |
| | | | to receptor 2 is not included in this |
| | | | quantity. |
| C_{SS, L1, disease} | Ligand 1 | 0.05 | The steady-state concentration of free |
| | CSS disease | | ligand 1 in the disease compartment in |
| | (nM) | | the absence of drug. Ligand 1 bound to |
| | | | receptor 1 is not included in this |
| | | | quantity. |
| C_{SS, L2, disease} | Ligand 2 | 0.05 | The steady-state concentration of free |
| | CSS disease | | ligand 2 in the disease compartment in |
| | (nM) | | the absence of drug. Ligand 2 bound to |
| | | | receptor 2 is not included in this |
| | | | quantity. |
| C_{SS, L1, tox} | Ligand 1 | 0.05 | The steady-state concentration of free |
| | CSS tox | | ligand 1 in the tox compartment in the |
| | (nM) | | absence of drug. Ligand 1 bound to |
| | | | receptor 1 is not included in this |
| | | | quantity. |
| C_{SS, L2, tox} | Ligand 2 | 0.05 | The steady-state concentration of free |
| | CSS tox | | ligand 2 in the tox compartment in the |
| | (nM) | | absence of drug. Ligand 2 bound to |
| | | | receptor 2 is not included in this |
| | | | quantity. |
| C_{SS, R1, central} | Receptor 1 | 10000 | The steady-state concentration of |
| | CSS central | | receptor 1 in the central compartment |
| | (#/cell) | | in the absence of drug. Receptor 1 |
| | | | bound to ligand 1 is included in this |
| | | | quantity, but soluble receptor 1 is not. |
| C_{SS, R2, central} | Receptor 2 | 10000 | The steady-state concentration of |
| | CSS central | | receptor 2 in the central compartment |
| | (#/cell) | | in the absence of drug. Receptor 2 |
| | | | bound to ligand 2 is included in this |
| | | | quantity, but soluble receptor 2 is not. |
| C_{SS, R1, peripheral} | Receptor 1 | 10000 | The steady-state concentration of |
| | CSS peripheral | | receptor 1 in the peripheral |
| | (#/cell) | | compartment in the absence of drug. |
| | | | Receptor 1 bound to ligand 1 is |
| | | | included in this quantity, but soluble |
| | | | receptor 1 is not. |
| C_{SS, R2, peripheral} | Receptor 2 | 10000 | The steady-state concentration of |
| | CSS peripheral | | receptor 2 in the peripheral |
| | (#/cell) | | compartment in the absence of drug. |
| | | | Receptor 2 bound to ligand 2 is |
| | | | included in this quantity, but soluble |
| | | | receptor 2 is not. |
| C_{SS, R1, disease} | Receptor 1 | 10000 | The steady-state concentration of |
| | CSS disease | | receptor 1 in the disease compartment |
| | (#/cell) | | in the absence of drug. Receptor 1 |
| | | | bound to ligand 1 is included in this |
| | | | quantity, but soluble receptor 1 is not. |
| C_{SS, R2, disease} | Receptor 2 | 10000 | The steady-state concentration of |
| | CSS disease | | receptor 2 in the disease compartment |
| | (#/cell) | | in the absence of drug. Receptor 2 |
| | | | bound to ligand 2 is included in this |
| | | | quantity, but soluble receptor 2 is not. |
| C_{SS, R1, tox} | Receptor 1 | 10000 | The steady-state concentration of |
| | CSS tox | | receptor 1 in the tox compartment in |
| | (#/cell) | | the absence of drug. Receptor 1 bound |
| | | | to ligand 1 is included in this quantity, |
| | | | but soluble receptor 1 is not. |
| C_{SS, R2, tox} | Receptor 2 | 10000 | The steady-state concentration of |
| | CSS tox | | receptor 2 in the tox compartment in |
| | (#/cell) | | the absence of drug. Receptor 2 bound |
| | | | to ligand 2 is included in this quantity, |
| | | | but soluble receptor 2 is not. |
| C_{SS, sR1, central} | Soluble | 0 | The steady-state concentration of |
| | receptor 1 | | soluble receptor 1 in the central |
| | CSS central | | compartment in the absence of drug. |
| | (nM) | | |
| C_{SS, sR2, central} | Soluble | 0 | The steady-state concentration of |
| | receptor 2 | | soluble receptor 2 in the central |
| | CSS central | | compartment in the absence of drug. |
| | (nM) | | |
| C_{SS, sR1, peripheral} | Soluble | 0 | The steady-state concentration of |
| | receptor 1 | | soluble receptor 1 in the peripheral |
| | CSS peripheral | | compartment in the absence of drug. |
| | (nM) | | |
| C_{SS, sR2, peripheral} | Soluble | 0 | The steady-state concentration of |
| | receptor 2 | | soluble receptor 2 in the peripheral |
| | CSS peripheral | | compartment in the absence of drug. |
| | (nM) | | |
| C_{SS, sR1, disease} | Soluble | 0 | The steady-state concentration of |
| | receptor 1 | | soluble receptor 1 in the disease |
| | CSS disease | | compartment in the absence of drug. |
| | (nM) | | |
| C_{SS, sR2, disease} | Soluble | 0 | The steady-state concentration of |
| | receptor 2 | | soluble receptor 2 in the disease |
| | CSS disease | | compartment in the absence of drug. |
| | (nM) | | |
| C_{SS, sR1, tox} | Soluble | 0 | The steady-state concentration of |
| | receptor 1 | | soluble receptor 1 in the tox |
| | CSS tox | | compartment in the absence of drug. |
| | (nM) | | |
| C_{SS, sR2, tox} | Soluble | 0 | The steady-state concentration of |
| | receptor 2 | | soluble receptor 2 in the tox |
| | CSS tox | | compartment in the absence of drug. |
| | (nM) | | |
| T_{dist, L1, peripheral} | Ligand 1 | 30 | The distribution rate of ligand 1 from |
| | Tdist | | the central compartment to the |
| | peripheral | | peripheral compartment. |
| | (hr) | | |
| T_{dist, L2, peripheral} | Ligand 2 | 30 | The distribution rate of ligand 2 from |
| | Tdist | | the central compartment to the |
| | peripheral | | peripheral compartment. |
| | (hr) | | |
| T_{dist, L1, disease} | Ligand 1 | 30 | The distribution rate of ligand 1 from |
| | Tdist disease | | the central compartment to the disease |
| | (hr) | | compartment. |
| T_{dist, L2, disease} | Ligand 2 | 30 | The distribution rate of ligand 2 from |
| | Tdist disease | | the central compartment to the disease |
| | (hr) | | compartment. |
| T_{dist, L1, tox} | Ligand 1 | 30 | The distribution rate of ligand 1 from |
| | Tdist tox | | the central compartment to the tox |
| | (hr) | | compartment. |
| T_{dist, L2, tox} | Ligand 2 | 30 | The distribution rate of ligand 2 from |
| | Tdist tox | | the central compartment to the tox |
| | (hr) | | compartment. |
| T_{dist, sR1, peripheral} | Soluble | 30 | The distribution rate of soluble receptor |
| | receptor | | 1 from the central compartment to the |
| | 1 Tdist | | peripheral compartment. |
| | peripheral (hr) | | |
| T_{dist, sR2, peripheral} | Soluble | 30 | The distribution rate of soluble receptor |
| | receptor | | 2 from the central compartment to the |
| | 2 Tdist | | peripheral compartment. |
| | peripheral (hr) | | |
| T_{dist, sR1, disease} | Soluble | 30 | The distribution rate of soluble receptor |
| | receptor | | 1 from the central compartment to the |
| | 1 Tdist | | disease compartment. |
| | disease (hr) | | |
| T_{dist, sR2, disease} | Soluble receptor | 30 | The distribution rate of soluble receptor |
| | 2 Tdist | | 2 from the central compartment to the |
| | disease (hr) | | disease compartment. |
| T_{dist, sR1, tox} | Soluble receptor | 30 | The distribution rate of soluble receptor |
| | 1 Tdist | | 1 from the central compartment to the |
| | tox (hr) | | tox compartment. |
| T_{dist, sR2, tox} | Soluble receptor | 30 | The distribution rate of soluble receptor |
| | 2 Tdist | | 2 from the central compartment to the |
| | tox (hr) | | tox compartment. |
| Density_{cells, central} | Cell density | 1000000 | Density of receptor-bearing cells in the |
| | central | | central compartment. |
| | (#/mL) | | |
| Density_{cells, peripheral} | Cell density | 1000000 | Density of receptor-bearing cells in the |
| | peripheral | | peripheral compartment. |
| | (#/mL) | | |
| Density_{cells, disease} | Cell density | 1000000 | Density of receptor-bearing cells in the |
| | disease | | disease compartment. |
| | (#/mL) | | |
| Density_{cells, tox} | Cell density | 1000000 | Density of receptor-bearing cells in the |
| | tox (#/mL) | | tox compartment. |
| Scale_{t1/2, R1, D:R1, central} | Drug: Receptor | 1 | A multiplicative factor for receptor 1 |
| | 1 T ½ | | turnover half-life when drug is bound |
| | scale central | | to it in the central compartment. Can be |
| | | | used to model effects the drug has on |
| | | | the internalization rate of this target. |
| Scale_{t1/2, R2, D:R2, central} | Drug: Receptor | 1 | A multiplicative factor for receptor 2 |
| | 2 T ½ | | turnover half-life when drug is bound |
| | scale central | | to it in the central compartment. Can be |
| | | | used to model effects the drug has on |
| | | | the internalization rate of this target. |
| Scale_{t1/2, R1, D:R1, peripheral} | Drug: Receptor | 1 | A multiplicative factor for receptor 1 |
| | 1 T ½ | | turnover half-life when drug is bound |
| | scale peripheral | | to it in the peripheral compartment. |
| | | | Can be used to model effects the drug |
| | | | has on the internalization rate of this |
| | | | target. |
| Scale_{t1/2, R2, D:R2, peripheral} | Drug: Receptor | 1 | A multiplicative factor for receptor 2 |
| | 2 T ½ | | turnover half-life when drug is bound |
| | scale peripheral | | to it in the peripheral compartment. |
| | | | Can be used to model effects the drug |
| | | | has on the internalization rate of this |
| | | | target. |
| Scale_{t1/2, R1, D:R1, disease} | Drug: Receptor | 1 | A multiplicative factor for receptor 1 |
| | 1 T ½ | | turnover half-life when drug is bound |
| | scale disease | | to it in the disease compartment. Can |
| | | | be used to model effects the drug has |
| | | | on the internalization rate of this target. |
| Scale_{t1/2, R2, D:R2, disease} | Drug: Receptor | 1 | A multiplicative factor for receptor 2 |
| | 2 T ½ | | turnover half-life when drug is bound |
| | scale disease | | to it in the disease compartment. Can |
| | | | be used to model effects the drug has |
| | | | on the internalization rate of this target. |
| Scale_{t1/2, R1, D:R1, tox} | Drug: Receptor | 1 | A multiplicative factor for receptor 1 |
| | 1 T ½ | | turnover half-life when drug is bound |
| | scale tox | | to it in the tox compartment. Can be |
| | | | used to model effects the drug has on |
| | | | the internalization rate of this target. |
| Scale_{t1/2, R2, D:R2, tox} | Drug: Receptor | 1 | A multiplicative factor for receptor 2 |
| | 2 T ½ | | turnover half-life when drug is bound |
| | scale tox | | to it in the tox compartment. Can be |
| | | | used to model effects the drug has on |
| | | | the internalization rate of this target. |
| Scale_{KD, R1, central} | Drug: Target | 1 | A multiplicative factor for the |
| | 1 Kd scale | | equilibrium dissociation constant |
| | central | | between drug and receptor 1 in the |
| | | | central compartment. Can be used to |
| | | | model drugs whose target affinity is |
| | | | affected by the compartment. |
| Scale_{KD, R2, central} | Drug: Target | 1 | A multiplicative factor for the |
| | 2 Kd scale | | equilibrium dissociation constant |
| | central | | between drug and receptor 2 in the |
| | | | central compartment. Can be used to |
| | | | model drugs whose target affinity is |
| | | | affected by the compartment. |
| Scale_{KD, R1, peripheral} | Drug: Target | 1 | A multiplicative factor for the |
| | 1 Kd scale | | equilibrium dissociation constant |
| | peripheral | | between drug and receptor 1 in the |
| | | | peripheral compartment. Can be used to |
| | | | model drugs whose target affinity is |
| | | | affected by the compartment. |
| Scale_{KD, R2, peripheral} | Drug: Target | 1 | A multiplicative factor for the |
| | 2 Kd scale | | equilibrium dissociation constant |
| | peripheral | | between drug and receptor 2 in the |
| | | | peripheral compartment. Can be used to |
| | | | model drugs whose target affinity is |
| | | | affected by the compartment. |
| Scale_{KD, R1, disease} | Drug: Target | 1 | A multiplicative factor for the |
| | 1 Kd scale | | equilibrium dissociation constant |
| | disease | | between drug and receptor 1 in the |
| | | | disease compartment. Can be used to |
| | | | model drugs whose target affinity is |
| | | | affected by the compartment. |
| Scale_{KD, R2, disease} | Drug: Target | 1 | A multiplicative factor for the |
| | 2 Kd scale | | equilibrium dissociation constant |
| | disease | | between drug and receptor 2 in the |
| | | | disease compartment. Can be used to |
| | | | model drugs whose target affinity is |
| | | | affected by the compartment. |
| Scale_{KD, R1, tox} | Drug: Target | 1 | A multiplicative factor for the |
| | 1 Kd scale | | equilibrium dissociation constant |
| | tox | | between drug and receptor 1 in the tox |
| | | | compartment. Can be used to model |
| | | | drugs whose target affinity is affected |
| | | | by the compartment. |
| Scale_{KD, R2, tox} | Drug: Target | 1 | A multiplicative factor for the |
| | 2 Kd scale | | equilibrium dissociation constant |
| | tox | | between drug and receptor 2 in the tox |
| | | | compartment. Can be used to model |
| | | | drugs whose target affinity is affected |
| | | | by the compartment. |
| Scale_{t1/2, central} | Soluble Drug | 1 | A multiplicative factor for the drug |
| | T ½ scale | | elimination half-life in the central |
| | central | | compartment. Can be used to model |
| | | | drugs which have a different half-life in |
| | | | some compartments. |
| Scale_{t1/2, peripheral} | Soluble Drug | 1 | A multiplicative factor for the drug |
| | T ½ scale | | elimination half-life in the peripheral |
| | peripheral | | compartment. Can be used to model |
| | | | drugs which have a different half-life in |
| | | | some compartments. |
| Scale_{t1/2, disease} | Soluble Drug | 1 | A multiplicative factor for the drug |
| | T ½ scale | | elimination half-life in the disease |
| | disease | | compartment. Can be used to model |
| | | | drugs which have a different half-life in |
| | | | some compartments. |
| Scale_{t1/2, tox} | Soluble Drug | 1 | A multiplicative factor for the drug |
| | T ½ scale | | elimination half-life in the tox |
| | tox | | compartment. Can be used to model |
| | | | drugs which have a different half-life in |
| | | | some compartments. |

TABLE 26
Example target criteria (e.g., for the therapeutic drug
candidate and the target for the therapeutic purpose)
of the example bispecific anti-receptor x anti-receptor
(4-compartment) model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free |
| Engagement | | | target due to drug binding. |
| | | | Measured as a percent of untreated. |
| Activation | 90 | percent | Percent of receptor occupied |
| | | | by ligand or drug. |
| | | | Measured as a percent of total. |
| | | | This is typically used |
| | | | for agonists. |

TABLE 27
Example output plots of the example bispecific anti-receptor ×
anti-receptor (4-compartment) model in the EFA system.

| Output Plot | Description |
|---|---|
| Criterion | The value of the selected criterion |
| vs scan | for each value of the 1D scan |
| parameter | parameter simulated. The desired |
| | target value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is any form of the |
| | drug not bound to any membrane proteins. |
| | This may include drug bound to one or |
| | more soluble proteins such as |
| | ligands or soluble receptors. |

In some implementations, the example bispecific anti-receptor x anti-receptor with avidity (4-compartment) model includes a four-compartment model of avid bivalent antibody binding to two different membrane receptors. A bispecific biotherapeutic that binds to two target cell surface receptors on a single cell type. For each receptor the drug either (1) acts as a competitive inhibitor by blocking the cognate-ligand from binding to its receptor or (2) acts as a receptor agonist. The molecule can be monovalent or bivalent with avidity for each target. This is a four-compartment model with a central, peripheral, disease and tox compartments. There is an option for +/− soluble receptor for each receptor. FIG. 15A shows a schematic diagram illustrating a biotherapeutic scenario of the example bispecific anti-receptor x anti-receptor with avidity (4-compartment) model, according to some embodiments. Example parameters of the example bispecific anti-receptor x anti-receptor with avidity (4-compartment) model in the EFA system are shown in Table 28. Example target criteria (e.g., for the therapeutic drug candidate and the target for the therapeutic purpose) of the example bispecific anti-receptor x anti-receptor with avidity (4-compartment) model in the EFA system are shown in Table 29. Example output plots of the example bispecific anti-receptor x anti-receptor with avidity (4-compartment) model in the EFA system are shown in Table 30.

TABLE 28
Example parameters of the example bispecific anti-receptor x anti-
receptor with avidity (4-compartment) model in the EFA system.

| Symbol | Name | Default | Definition |
|---|---|---|---|
| τ | Interval | 14 | The interval between doses of the drug. |
| D | Dose (mg) | 100 | The amount of each dose. |
| K_{D, R1} | Drug: | 0.1 | The affinity of the drug for receptor 1 as |
| | Target 1 KD | | characterized by the equilibrium dissociation |
| | (nM) | | constant. This is the monovalent affinity (i.e. the |
| | | | affinity of single binding). |
| K_{D, R2} | Drug: | 0.1 | The affinity of the drug for receptor 2 as |
| | Target 2 KD | | characterized by the equilibrium dissociation |
| | (nM) | | constant. This is the monovalent affinity (i.e. the |
| | | | affinity of single binding). |
| N_{doses} | Number of | 7 | The number of doses to be given. |
| | doses | | |
| MW | Molecular | 150000 | The molecular weight of the drug. This is used to |
| | Weight | | convert the dose which is expressed in mass units to |
| | (Da) | | a molar dose. |
| t_{1/2} | Biologic | 28 | The first-order elimination rate of the drug. |
| | first order | | |
| | T ½ | | |
| | (days) | | |
| t_{1/2, a} | SC | 2.5 | The first-order absorption rate of the drug from the |
| | absorption | | subcutaneous compartment into the central |
| | T ½ | | compartment. |
| | (days) | | |
| BW | Body | 70 | The body weight of the subject. This is used to |
| | Weight | | convert the dose when expressed in units of mg/kg. |
| | (kg) | | |
| V | Volume | 2.5 | The volume of the central compartment which is |
| | central | | typically the plasma of the peripheral blood. |
| | (L) | | |
| Vₚₑᵣᵢₚₕₑᵣₐₗ | Volume | 12.8 | The volume of the non-blood fluids that the |
| | peripheral | | antibody can distribute to. |
| | (L) | | |
| V_{disease} | Volume | 0.1 | The volume of the disease compartment interstitial |
| | disease | | fluid. |
| | (L) | | |
| Vₜₒₓ | Volume | 0.1 | The volume of the tox compartment interstitial |
| | tox | | fluid. |
| | (L) | | |
| T_{dist, peripheral} | Drug | 30 | The distribution rate of drug from the central |
| | Tdist | | compartment to the peripheral compartment. |
| | peripheral | | |
| | (hr) | | |
| T_{dist, disease} | Drug | 30 | The distribution rate of drug from the central |
| | Tdist | | compartment to the disease compartment. |
| | disease | | |
| | (hr) | | |
| T_{dist, tox} | Drug | 30 | The distribution rate of drug from the central |
| | Tdist | | compartment to the tox compartment. |
| | tox | | |
| | (hr) | | |
| P_{dist, peripheral} | Drug | 0.190625 | The steady-state ratio of soluble drug in the |
| | partition | | peripheral compartment to soluble drug in the |
| | coefficient | | central compartment. |
| | peripheral | | |
| P_{dist, disease} | Drug | 0.3 | The steady-state ratio of soluble drug in the disease |
| | partition | | compartment to soluble drug in the central |
| | coefficient | | compartment. |
| | disease | | |
| P_{dist, tox} | Drug | 0.3 | The steady-state ratio of soluble drug in the tox |
| | partition | | compartment to soluble drug in the central |
| | coefficient | | compartment. |
| | tox | | |
| Valency₁ | Effective | 2 | The number of receptor 1 molecules that a molecule |
| | Valency 1 | | of drug can bind. This may be 1 or 2. |
| Valency₂ | Effective | 2 | The number of receptor 2 molecules that a molecule |
| | Valency 2 | | of drug can bind. This may be 1 or 2. |
| χ° | Avidity | 1 | The chi factor of a standard assay. It is the observed |
| | chi | | chi factor of an assay with with 1e6 cells/mL with a |
| | factor | | cell diameter of 10 urn. |
| t_{1/2, L1} | Ligand 1 | 0.5 | The first-order elimination rate of ligand 1 from the |
| | T ½ | | central compartment. |
| | (hr) | | |
| t_{1/2, L2} | Ligand 2 | 0.5 | The first-order elimination rate of ligand 2 from the |
| | T ½ | | central compartment. |
| | (hr) | | |
| t_{1/2, R1} | Receptor | 1 | The first-order turnover rate of receptor 1 in all |
| | 1 T ½ | | compartments. |
| | (hr) | | |
| t_{1/2, R2} | Receptor | 1 | The first-order turnover rate of receptor 2 in all |
| | 2 T ½ | | compartments. |
| | (hr) | | |
| t_{1/2, sR1} | Soluble | 0.5 | The first-order elimination rate of soluble receptor 1 |
| | receptor 1 | | from the central compartment. |
| | T ½ | | |
| | (hr) | | |
| t_{1/2, sR2} | Soluble | 0.5 | The first-order elimination rate of soluble receptor 2 |
| | receptor 2 | | from the central compartment. |
| | T ½ | | |
| | (hr) | | |
| K_{D, L1:R1} | L1:R1 | 1 | The binding affinity of ligand 1 for receptor 1 as |
| | Affinity | | characterized by the equilibrium dissociation |
| | (nM) | | constant. |
| K_{D, L2:R2} | L2:R2 | 1 | The binding affinity of ligand 2 for receptor 2 as |
| | Affinity | | characterized by the equilibrium dissociation |
| | (nM) | | constant. |
| C_{SS, L1, central} | Ligand 1 | 0.05 | The steady-state concentration of free ligand 1 in |
| | CSS | | the central compartment in the absence of drug. |
| | central | | Ligand 1 bound to receptor 1 is not included in this |
| | (nM) | | quantity. |
| C_{SS, L2, central} | Ligand 2 | 0.05 | The steady-state concentration of free ligand 2 in |
| | CSS | | the central compartment in the absence of drug. |
| | central | | Ligand 2 bound to receptor 2 is not included in this |
| | (nM) | | quantity. |
| C_{SS, L1, peripheral} | Ligand 1 | 0.05 | The steady-state concentration of free ligand 1 in |
| | CSS | | the peripheral compartment in the absence of drug. |
| | peripheral | | Ligand 1 bound to receptor 1 is not included in this |
| | (nM) | | quantity. |
| C_{SS, L2, peripheral} | Ligand 2 | 0.05 | The steady-state concentration of free ligand 2 in |
| | CSS | | the peripheral compartment in the absence of drug. |
| | peripheral | | Ligand 2 bound to receptor 2 is not included in this |
| | (nM) | | quantity. |
| C_{SS, L1, disease} | Ligand 1 | 0.05 | The steady-state concentration of free ligand 1 in |
| | CSS | | the disease compartment in the absence of drug. |
| | disease | | Ligand 1 bound to receptor 1 is not included in this |
| | (nM) | | quantity. |
| C_{SS, L2, disease} | Ligand 2 | 0.05 | The steady-state concentration of free ligand 2 in |
| | CSS | | the disease compartment in the absence of drug. |
| | disease | | Ligand 2 bound to receptor 2 is not included in this |
| | (nM) | | quantity. |
| C_{SS, L1, tox} | Ligand 1 | 0.05 | The steady-state concentration of free ligand 1 in |
| | CSS | | the tox compartment in the absence of drug. Ligand |
| | tox | | 1 bound to receptor 1 is not included in this |
| | (nM) | | quantity. |
| C_{SS, L2, tox} | Ligand 2 | 0.05 | The steady-state concentration of free ligand 2 in |
| | CSS | | the tox compartment in the absence of drug. Ligand |
| | tox | | 2 bound to receptor 2 is not included in this |
| | (nM) | | quantity. |
| C_{SS, R1, central} | Receptor | 10000 | The steady-state concentration of receptor 1 in the |
| | 1 CSS | | central compartment in the absence of drug. |
| | central | | Receptor 1 bound to ligand 1 is included in this |
| | (#/cell) | | quantity, but soluble receptor 1 is not. |
| C_{SS, R2, central} | Receptor | 10000 | The steady-state concentration of receptor 2 in the |
| | 2 CSS | | central compartment in the absence of drug. |
| | central | | Receptor 2 bound to ligand 2 is included in this |
| | (#/cell) | | quantity, but soluble receptor 2 is not. |
| C_{SS, R1, peripheral} | Receptor | 10000 | The steady-state concentration of receptor 1 in the |
| | 1 CSS | | peripheral compartment in the absence of drug. |
| | peripheral | | Receptor 1 bound to ligand 1 is included in this |
| | (#/cell) | | quantity, but soluble receptor 1 is not. |
| C_{SS, R2, peripheral} | Receptor | 10000 | The steady-state concentration of receptor 2 in the |
| | 2 CSS | | peripheral compartment in the absence of drug. |
| | peripheral | | Receptor 2 bound to ligand 2 is included in this |
| | (#/cell) | | quantity, but soluble receptor 2 is not. |
| C_{SS, R1, disease} | Receptor | 10000 | The steady-state concentration of receptor 1 in the |
| | 1 CSS | | disease compartment in the absence of drug. |
| | disease | | Receptor 1 bound to ligand 1 is included in this |
| | (#/cell) | | quantity, but soluble receptor 1 is not. |
| C_{SS, R2, disease} | Receptor | 10000 | The steady-state concentration of receptor 2 in the |
| | 2 CSS | | disease compartment in the absence of drug. |
| | disease | | Receptor 2 bound to ligand 2 is included in this |
| | (#/cell) | | quantity, but soluble receptor 2 is not. |
| C_{SS, R1, tox} | Receptor | 10000 | The steady-state concentration of receptor 1 in the |
| | 1 CSS tox | | tox compartment in the absence of drug. Receptor 1 |
| | (#/cell) | | bound to ligand 1 is included in this quantity, but |
| | | | soluble receptor 1 is not. |
| C_{SS, R2, tox} | Receptor | 10000 | The steady-state concentration of receptor 2 in the |
| | 2 CSS tox | | tox compartment in the absence of drug. Receptor 2 |
| | (#/cell) | | bound to ligand 2 is included in this quantity, but |
| | | | soluble receptor 2 is not. |
| C_{SS, sR1, central} | Soluble | 0 | The steady-state concentration of soluble receptor 1 |
| | receptor 1 | | in the central compartment in the absence of drug. |
| | CSS | | |
| | central | | |
| | (nM) | | |
| C_{SS, sR2, central} | Soluble | 0 | The steady-state concentration of soluble receptor 2 |
| | receptor 2 | | in the central compartment in the absence of drug. |
| | CSS | | |
| | central | | |
| | (nM) | | |
| C_{SS, sR1, peripheral} | Soluble | 0 | The steady-state concentration of soluble receptor 1 |
| | receptor 1 | | in the peripheral compartment in the absence of |
| | CSS | | drug. |
| | peripheral | | |
| | (nM) | | |
| C_{SS, sR2, peripheral} | Soluble | 0 | The steady-state concentration of soluble receptor 2 |
| | receptor 2 | | in the peripheral compartment in the absence of |
| | CSS | | drug. |
| | peripheral | | |
| | (nM) | | |
| C_{SS, sR1, disease} | Soluble | 0 | The steady-state concentration of soluble receptor 1 |
| | receptor 1 | | in the disease compartment in the absence of drug. |
| | CSS | | |
| | disease | | |
| | (nM) | | |
| C_{SS, sR2, disease} | Soluble | 0 | The steady-state concentration of soluble receptor 2 |
| | receptor 2 | | in the disease compartment in the absence of drug. |
| | CSS | | |
| | disease | | |
| | (nM) | | |
| C_{SS, sR1, tox} | Soluble | 0 | The steady-state concentration of soluble receptor 1 |
| | receptor 1 | | in the tox compartment in the absence of drug. |
| | CSS tox | | |
| | (nM) | | |
| C_{SS, sR2, tox} | Soluble | 0 | The steady-state concentration of soluble receptor 2 |
| | receptor 2 | | in the tox compartment in the absence of drug. |
| | CSS tox | | |
| | (nM) | | |
| T_{dist, L1, peripheral} | Ligand 1 | 30 | The distribution rate of ligand 1 from the central |
| | Tdist | | compartment to the peripheral compartment. |
| | peripheral | | |
| | (hr) | | |
| T_{dist, L2, peripheral} | Ligand 2 | 30 | The distribution rate of ligand 2 from the central |
| | Tdist | | compartment to the peripheral compartment. |
| | peripheral | | |
| | (hr) | | |
| T_{dist, L1, disease} | Ligand 1 | 30 | The distribution rate of ligand 1 from the central |
| | Tdist | | compartment to the disease compartment. |
| | disease | | |
| | (hr) | | |
| T_{dist, L2, disease} | Ligand 2 | 30 | The distribution rate of ligand 2 from the central |
| | Tdist | | compartment to the disease compartment. |
| | disease | | |
| | (hr) | | |
| T_{dist, L1, tox} | Ligand 1 | 30 | The distribution rate of ligand 1 from the central |
| | Tdist | | compartment to the tox compartment. |
| | tox | | |
| | (hr) | | |
| T_{dist, L2, tox} | Ligand 2 | 30 | The distribution rate of ligand 2 from the central |
| | Tdist tox | | compartment to the tox compartment. |
| | (hr) | | |
| T_{dist, sR1, peripheral} | Soluble | 30 | The distribution rate of soluble receptor 1 from the |
| | receptor 1 | | central compartment to the peripheral compartment. |
| | Tdist | | |
| | peripheral | | |
| | (hr) | | |
| T_{dist, sR2, peripheral} | Soluble | 30 | The distribution rate of soluble receptor 2 from the |
| | receptor 2 | | central compartment to the peripheral compartment. |
| | Tdist | | |
| | peripheral | | |
| | (hr) | | |
| T_{dist, sR1, disease} | Soluble | 30 | The distribution rate of soluble receptor 1 from the |
| | receptor 1 | | central compartment to the disease compartment. |
| | Tdist | | |
| | disease | | |
| | (hr) | | |
| T_{dist, sR2, disease} | Soluble | 30 | The distribution rate of soluble receptor 2 from the |
| | receptor 2 | | central compartment to the disease compartment. |
| | Tdist | | |
| | disease | | |
| | (hr) | | |
| T_{dist, sR1, tox} | Soluble | 30 | The distribution rate of soluble receptor 1 from the |
| | receptor 1 | | central compartment to the tox compartment. |
| | Tdist | | |
| | tox | | |
| | (hr) | | |
| T_{dist, sR2, tox} | Soluble | 30 | The distribution rate of soluble receptor 2 from the |
| | receptor 2 | | central compartment to the tox compartment. |
| | Tdist | | |
| | tox | | |
| | (hr) | | |
| Diam_{cell} | Cell | 10 | Diameter of receptor-bearing cells. |
| | diameter | | |
| | (um) | | |
| Density_{cells, central} | Cell | 1000000 | Density of receptor-bearing cells in the central |
| | density | | compartment. |
| | central | | |
| | (#/mL) | | |
| Density_{cells, peripheral} | Cell | 1000000 | Density of receptor-bearing cells in the peripheral |
| | density | | compartment. |
| | peripheral | | |
| | (#/mL) | | |
| Density_{cells, disease} | Cell | 1000000 | Density of receptor-bearing cells in the disease |
| | density | | compartment. |
| | disease | | |
| | (#/mL) | | |
| Density_{cells, tox} | Cell | 1000000 | Density of receptor-bearing cells in the tox |
| | density | | compartment. |
| | tox | | |
| | (#/mL) | | |
| Scale_{t1/2, R1, D:R1, central} | Drug: | 1 | A multiplicative factor for receptor 1 turnover half- |
| | Receptor 1 | | life when drug is bound to it in the central |
| | T ½ scale | | compartment. Can be used to model effects the drug |
| | central | | has on the internalization rate of this target. |
| Scale_{t1/2, R2, D:R2, central} | Drug: | 1 | A multiplicative factor for receptor 2 turnover half- |
| | Receptor 2 | | life when drug is bound to it in the central |
| | T ½ scale | | compartment. Can be used to model effects the drug |
| | central | | has on the internalization rate of this target. |
| Scale_{t1/2, R1, D:R1, peripheral} | Drug: | 1 | A multiplicative factor for receptor 1 turnover half- |
| | Receptor 1 | | life when drug is bound to it in the peripheral |
| | T ½ scale | | compartment. Can be used to model effects the drug |
| | peripheral | | has on the internalization rate of this target. |
| Scale_{t1/2, R2, D:R2, peripheral} | Drug: | 1 | A multiplicative factor for receptor 2 turnover half- |
| | Receptor 2 | | life when drug is bound to it in the peripheral |
| | T ½ scale | | compartment. Can be used to model effects the drug |
| | peripheral | | has on the internalization rate of this target. |
| Scale_{t1/2, R1, D:R1, disease} | Drug: | 1 | A multiplicative factor for receptor 1 turnover half- |
| | Receptor 1 | | life when drug is bound to it in the disease |
| | T ½ scale | | compartment. Can be used to model effects the drug |
| | disease | | has on the internalization rate of this target. |
| Scale_{t1/2, R2, D:R2, disease} | Drug: | 1 | A multiplicative factor for receptor 2 turnover half- |
| | Receptor 2 | | life when drug is bound to it in the disease |
| | T ½ scale | | compartment. Can be used to model effects the drug |
| | disease | | has on the internalization rate of this target. |
| Scale_{t1/2, R1, D:R1, tox} | Drug: | 1 | A multiplicative factor for receptor 1 turnover half- |
| | Receptor 1 | | life when drug is bound to it in the tox |
| | T ½ scale | | compartment. Can be used to model effects the drug |
| | tox | | has on the internalization rate of this target. |
| Scale_{t1/2, R2, D:R2, tox} | Drug: | 1 | A multiplicative factor for receptor 2 turnover half- |
| | Receptor 2 | | life when drug is bound to it in the tox |
| | T ½ scale | | compartment. Can be used to model effects the drug |
| | tox | | has on the internalization rate of this target. |
| Scale_{KD, R1, central} | Drug: | 1 | A multiplicative factor for the equilibrium |
| | Target 1 | | dissociation constant between drug and receptor 1 |
| | Kd scale | | in the central compartment. Can be used to model |
| | central | | drugs whose target affinity is affected by the |
| | | | compartment. |
| Scale_{KD, R2, central} | Drug: | 1 | A multiplicative factor for the equilibrium |
| | Target 2 | | dissociation constant between drug and receptor 2 |
| | Kd scale | | in the central compartment. Can be used to model |
| | central | | drugs whose target affinity is affected by the |
| | | | compartment. |
| Scale_{KD, R1, peripheral} | Drug: | 1 | A multiplicative factor for the equilibrium |
| | Target 1 | | dissociation constant between drug and receptor 1 |
| | Kd scale | | in the peripheral compartment. Can be used to |
| | peripheral | | model drugs whose target affinity is affected by the |
| | | | compartment. |
| Scale_{KD, R2, peripheral} | Drug: | 1 | A multiplicative factor for the equilibrium |
| | Target 2 | | dissociation constant between drug and receptor 2 |
| | Kd scale | | in the peripheral compartment. Can be used to |
| | peripheral | | model drugs whose target affinity is affected by the |
| | | | compartment. |
| Scale_{KD, R1, disease} | Drug: | 1 | A multiplicative factor for the equilibrium |
| | Target 1 | | dissociation constant between drug and receptor 1 |
| | Kd scale | | in the disease compartment. Can be used to model |
| | disease | | drugs whose target affinity is affected by the |
| | | | compartment. |
| Scale_{KD, R2, disease} | Drug: | 1 | A multiplicative factor for the equilibrium |
| | Target 2 | | dissociation constant between drug and receptor 2 |
| | Kd scale | | in the disease compartment. Can be used to model |
| | disease | | drugs whose target affinity is affected by the |
| | | | compartment. |
| Scale_{KD, R1, tox} | Drug: | 1 | A multiplicative factor for the equilibrium |
| | Target 1 | | dissociation constant between drug and receptor 1 |
| | Kd scale | | in the tox compartment. Can be used to model drugs |
| | tox | | whose target affinity is affected by the |
| | | | compartment. |
| Scale_{KD, R2, tox} | Drug: | 1 | A multiplicative factor for the equilibrium |
| | Target 2 | | dissociation constant between drug and receptor 2 |
| | Kd scale | | in the tox compartment. Can be used to model drugs |
| | tox | | whose target affinity is affected by the |
| | | | compartment. |
| Scale_{t1/2, central} | Soluble | 1 | A multiplicative factor for the drug elimination |
| | Drug T | | half-life in the central compartment. Can be used to |
| | ½ scale | | model drugs which have a different half-life in |
| | central | | some compartments. |
| Scale_{t1/2, peripheral} | Soluble | 1 | A multiplicative factor for the drug elimination |
| | Drug T | | half-life in the peripheral compartment. Can be used |
| | ½ scale | | to model drugs which have a different half-life in |
| | peripheral | | some compartments. |
| Scale_{t1/2, disease} | Soluble | 1 | A multiplicative factor for the drug elimination |
| | Drug T | | half-life in the disease compartment. Can be used to |
| | ½ scale | | model drugs which have a different half-life in |
| | disease | | some compartments. |
| Scale_{t1/2, tox} | Soluble | 1 | A multiplicative factor for the drug elimination |
| | Drug T | | half-life in the tox compartment. Can be used to |
| | ½ scale | | model drugs which have a different half-life in |
| | tox | | some compartments. |

TABLE 29
Example target criteria (e.g., for the therapeutic drug
candidate and the target for the therapeutic purpose)
of the example bispecific anti-receptor × anti-receptor
with avidity (4-compartment) model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free |
| Engagement | | | target due to drug binding. |
| | | | Measured as a percent of untreated. |
| Activation | 90 | percent | Percent of receptor occupied |
| | | | by ligand or drug. |
| | | | Measured as a percent of |
| | | | total. This is typically used |
| | | | for agonists. |

TABLE 30
Example output plots of the example bispecific anti-receptor ×
anti-receptor with avidity (4-compartment) model in the EFA system.

| Output Plot | Description |
|---|---|
| Criterion | The value of the selected criterion |
| vs scan | for each value of the 1D scan |
| parameter | parameter simulated. The desired |
| | target value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is any form of the |
| | drug not bound to any membrane proteins. This |
| | may include drug bound to one or more |
| | soluble proteins such as |
| | ligands or soluble receptors. |

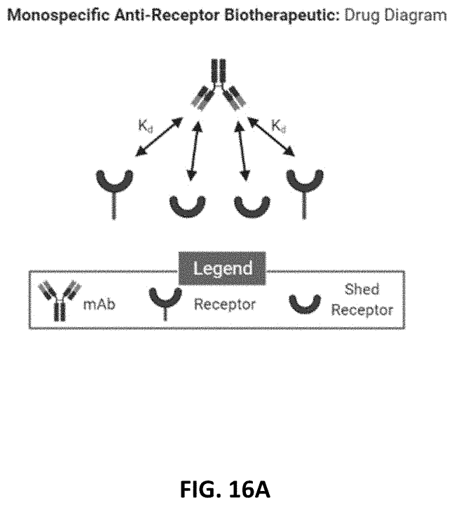

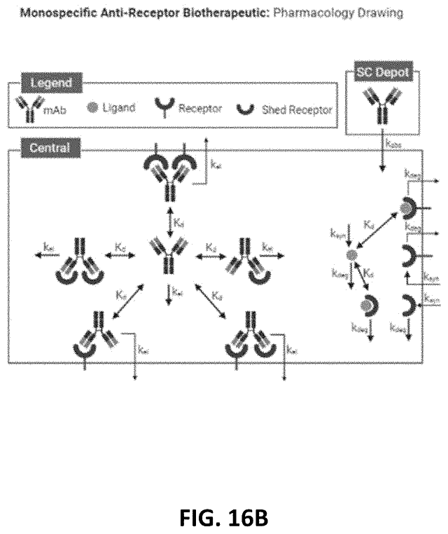

In some implementations, the example monospecific anti-receptor model includes a one-compartment model of bivalent antibody binding to membrane receptor. A biologic that binds to a cell surface receptor target and either (1) acts as a competitive inhibitor by blocking the cognate-ligand from binding to its receptor or (2) acts as a receptor agonist. The molecule can be mono- or bivalent. This is a one-compartment model and there is an option for +/− soluble receptor. FIGS. 16A-16B show schematic diagrams illustrating a biotherapeutic scenario of the example monospecific anti-receptor model, according to some embodiments. Example parameters of the example monospecific anti-receptor model in the EFA system are shown in Table 31. Example target criteria (e.g., for the therapeutic drug candidate and the target for the therapeutic purpose) of the example monospecific anti-receptor model in the EFA system are shown in Table 32. Example output plots of the example monospecific anti-receptor model in the EFA system are shown in Table 33.

TABLE 31
Example parameters of the example monospecific anti-receptor model in the EFA system.

| Symbol | Name | Default | Definition |
|---|---|---|---|
| τ | Interval | 14 | The interval between doses of the drug. |
| D | Dose | 100 | The amount of each dose. |
| | (mg) | | |
| K_{D, R} | Drug: | 0.1 | The affinity of the drug for receptor as characterized |
| | Target | | by the equilibrium dissociation constant. This is the |
| | KD | | monovalent affinity (i.e. the affinity of single |
| | (nM) | | binding). |
| N_{doses} | Number of | 7 | The number of doses to be given. |
| | doses | | |
| MW | Molecular | 150000 | The molecular weight of the drug. This is used to |
| | Weight | | convert the dose which is expressed in mass units to a |
| | (Da) | | molar dose. |
| t_{1/2} | Biologic | 28 | The first-order elimination rate of the drug. |
| | first | | |
| | order | | |
| | T ½ | | |
| | (days) | | |
| t_{1/2, a} | SC | 2.5 | The first-order absorption rate of the drug from the |
| | absorption | | subcutaneous compartment into the central |
| | T ½ | | compartment. |
| | (days) | | |
| BW | Body | 70 | The body weight of the subject. This is used to |
| | Weight | | convert the dose when expressed in units of mg/kg. |
| | (kg) | | |
| V | Volume | 5 | The volume of the central compartment which is |
| | (L) | | typically the plasma of the peripheral blood. |
| Valency | Effective | 2 | The number of receptor molecules that a molecule of |
| | Valency | | drug can bind. This may be 1 or 2. |
| t_{1/2, L} | Ligand | 0.5 | The first-order elimination rate of ligand from the |
| | T ½ | | central compartment. |
| | (hr) | | |
| t_{1/2, R} | Receptor | 1 | The first-order turnover rate of receptor in all |
| | T ½ | | compartments. |
| | (hr) | | |
| t_{1/2, sR} | Soluble | 0.5 | The first-order elimination rate of soluble receptor |
| | receptor | | from the central compartment. |
| | T ½ | | |
| | (hr) | | |
| K_{D, L:R} | L:R | 1 | The binding affinity of ligand for receptor as |
| | Affinity | | characterized by the equilibrium dissociation |
| | (nM) | | constant. |
| C_{SS, L} | Ligand | 0.05 | The steady-state concentration of free ligand in the |
| | CSS | | compartment in the absence of drug. Ligand bound to |
| | (nM) | | receptor is not included in this quantity. |
| C_{SS, R} | Receptor | 10000 | The steady-state concentration of receptor in the |
| | CSS | | compartment in the absence of drug. Receptor bound |
| | (#/cell) | | to ligand is included in this quantity, but soluble |
| | | | receptor is not. |
| C_{SS, sR} | Soluble | 0 | The steady-state concentration of soluble receptor in |
| | receptor | | the compartment in the absence of drug. |
| | CSS | | |
| | (nM) | | |
| Density_{cells} | Cell | 1000000 | Density of receptor-bearing cells in the compartment. |
| | density | | |
| | (#/mL) | | |

TABLE 32
Example target criteria (e.g., for the therapeutic drug candidate
and the target for the therapeutic purpose) of the example
monospecific anti-receptor model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free target |
| Engagement | | | due to drug binding. |
| | | | Measured as a percent of untreated. |
| Activation | 90 | percent | Percent of receptor occupied |
| | | | by ligand or drug. |
| | | | Measured as a percent of total. |
| | | | This is typically used |
| | | | for agonists. |

TABLE 33
Example output plots of the example monospecific
anti-receptor model in the EFA system.

| Output Plot | Description |
|---|---|
| Criterion | The value of the selected criterion |
| vs scan | for each value of the 1D scan |
| parameter | parameter simulated. The desired |
| | target value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the |
| | concentration of drug not bound to anything. |
| Total drug | A time course plot of the concentration |
| | of soluble drug. This is any form |
| | of the drug not bound to any membrane |
| | proteins. This may include drug bound to |
| | one or more soluble proteins such as |
| | ligands or soluble receptors. |

In some implementations, the example monospecific anti-receptor (4-compartment) model includes a four-compartment model of bivalent antibody binding to membrane receptor. A biologic that binds to a cell surface receptor target and either (1) acts as a competitive inhibitor by blocking the cognate-ligand from binding to its receptor or (2) acts as a receptor agonist. The molecule can be mono- or bivalent. This is a four-compartment model with a central, peripheral, disease and tox compartments. There is an option for +/− soluble receptor. FIG. 16A shows a schematic diagram illustrating a biotherapeutic scenario of the example monospecific anti-receptor (4-compartment) model, according to some embodiments. Example parameters of the example monospecific anti-receptor (4-compartment) model in the EFA system are shown in Table 34. Example target criteria (e.g., for the therapeutic drug candidate and the target for the therapeutic purpose) of the example monospecific anti-receptor (4-compartment) model in the EFA system are shown in Table 35. Example output plots of the example monospecific anti-receptor (4-compartment) model in the EFA system are shown in Table 36.

TABLE 34
Example parameters of the example monospecific anti-
receptor (4-compartment) model in the EFA system.

| Symbol | Name | Default | Definition |
|---|---|---|---|
| τ | Interval | 14 | The interval between doses of the drug. |
| D | Dose | 100 | The amount of each dose. |
| | (mg) | | |
| K_{D, R} | Drug: | 0.1 | The affinity of the drug for receptor as characterized |
| | Target | | by the equilibrium dissociation constant. This is the |
| | KD | | monovalent affinity (i.e. the affinity of single |
| | (nM) | | binding). |
| N_{doses} | Number of | 7 | The number of doses to be given. |
| | doses | | |
| MW | Molecular | 150000 | The molecular weight of the drug. This is used to |
| | Weight | | convert the dose which is expressed in mass units to |
| | (Da) | | a molar dose. |
| t_{1/2} | Biologic | 28 | The first-order elimination rate of the drug. |
| | first | | |
| | order | | |
| | T ½ | | |
| | (days) | | |
| t_{1/2, a} | SC | 2.5 | The first-order absorption rate of the drug from the |
| | absorption | | subcutaneous compartment into the central |
| | T ½ | | compartment. |
| | (days) | | |
| BW | Body | 70 | The body weight of the subject. This is used to |
| | Weight | | convert the dose when expressed in units of mg/kg. |
| | (kg) | | |
| V | Volume | 2.5 | The volume of the central compartment which is |
| | central | | typically the plasma of the peripheral blood. |
| | (L) | | |
| Vₚₑᵣᵢₚₕₑᵣₐₗ | Volume | 12.8 | The volume of the non-blood fluids that the antibody |
| | peripheral | | can distribute to. |
| | (L) | | |
| V_{disease} | Volume | 0.1 | The volume of the disease compartment interstitial |
| | disease | | fluid. |
| | (L) | | |
| Vₜₒₓ | Volume | 0.1 | The volume of the tox compartment interstitial fluid. |
| | tox | | |
| | (L) | | |
| T_{dist, peripheral} | Drug | 30 | The distribution rate of drug from the central |
| | Tdist | | compartment to the peripheral compartment. |
| | peripheral | | |
| | (hr) | | |
| T_{dist, disease} | Drug | 30 | The distribution rate of drug from the central |
| | Tdist | | compartment to the disease compartment. |
| | disease | | |
| | (hr) | | |
| T_{dist, tox} | Drug | 30 | The distribution rate of drug from the central |
| | Tdist | | compartment to the tox compartment. |
| | tox | | |
| | (hr) | | |
| P_{dist, peripheral} | Drug | 0.190625 | The steady-state ratio of soluble drug in the |
| | partition | | peripheral compartment to soluble drug in the central |
| | coefficient | | compartment. |
| | peripheral | | |
| P_{dist, disease} | Drug | 0.3 | The steady-state ratio of soluble drug in the disease |
| | partition | | compartment to soluble drug in the central |
| | coefficient | | compartment. |
| | disease | | |
| P_{dist, tox} | Drug | 0.3 | The steady-state ratio of soluble drug in the tox |
| | partition | | compartment to soluble drug in the central |
| | coefficient | | compartment. |
| | tox | | |
| Valency | Effective | 2 | The number of receptor molecules that a molecule of |
| | Valency | | drug can bind. This may be 1 or 2. |
| t_{1/2, L} | Ligand | 0.5 | The first-order elimination rate of ligand from the |
| | T ½ | | central compartment. |
| | (hr) | | |
| t_{1/2, R} | Receptor | 1 | The first-order turnover rate of receptor in all |
| | T ½ | | compartments. |
| | (hr) | | |
| t_{1/2, sR} | Soluble | 0.5 | The first-order elimination rate of soluble receptor |
| | receptor | | from the central compartment. |
| | T ½ | | |
| | (hr) | | |
| K_{D, L:R} | L:R | 1 | The binding affinity of ligand for receptor as |
| | Affinity | | characterized by the equilibrium dissociation |
| | (nM) | | constant. |
| C_{SS, L, central} | Ligand | 0.05 | The steady-state concentration of free ligand in the |
| | CSS | | central compartment in the absence of drug. Ligand |
| | central | | bound to receptor is not included in this quantity. |
| | (nM) | | |
| C_{SS, L, peripheral} | Ligand | 0.05 | The steady-state concentration of free ligand in the |
| | CSS | | peripheral compartment in the absence of drug. |
| | peripheral | | Ligand bound to receptor is not included in this |
| | (nM) | | quantity. |
| C_{SS, L, disease} | Ligand | 0.05 | The steady-state concentration of free ligand in the |
| | CSS | | disease compartment in the absence of drug. Ligand |
| | disease | | bound to receptor is not included in this quantity. |
| | (nM) | | |
| C_{SS, L, tox} | Ligand | 0.05 | The steady-state concentration of free ligand in the |
| | CSS | | tox compartment in the absence of drug. Ligand |
| | tox | | bound to receptor is not included in this quantity. |
| | (nM) | | |
| C_{SS, R, central} | Receptor | 10000 | The steady-state concentration of receptor in the |
| | CSS | | central compartment in the absence of drug. |
| | central | | Receptor bound to ligand is included in this quantity, |
| | (#/cell) | | but soluble receptor is not. |
| C_{SS, R, peripheral} | Receptor | 10000 | The steady-state concentration of receptor in the |
| | CSS | | peripheral compartment in the absence of drug. |
| | peripheral | | Receptor bound to ligand is included in this quantity, |
| | (#/cell) | | but soluble receptor is not. |
| C_{SS, R, disease} | Receptor | 10000 | The steady-state concentration of receptor in the |
| | CSS | | disease compartment in the absence of drug. |
| | disease | | Receptor bound to ligand is included in this quantity, |
| | (#/cell) | | but soluble receptor is not. |
| C_{SS, R, tox} | Receptor | 10000 | The steady-state concentration of receptor in the tox |
| | CSS | | compartment in the absence of drug. Receptor bound |
| | tox | | to ligand is included in this quantity, but soluble |
| | (#/cell) | | receptor is not. |
| C_{SS, sR, central} | Soluble | 0 | The steady-state concentration of soluble receptor in |
| | receptor | | the central compartment in the absence of drug. |
| | CSS | | |
| | central | | |
| | (nM) | | |
| C_{SS, sR, peripheral} | Soluble | 0 | The steady-state concentration of soluble receptor in |
| | receptor | | the peripheral compartment in the absence of drug. |
| | CSS | | |
| | peripheral | | |
| | (nM) | | |
| C_{SS, sR, disease} | Soluble | 0 | The steady-state concentration of soluble receptor in |
| | receptor | | the disease compartment in the absence of drug. |
| | CSS | | |
| | disease | | |
| | (nM) | | |
| C_{SS, sR, tox} | Soluble | 0 | The steady-state concentration of soluble receptor in |
| | receptor | | the tox compartment in the absence of drug. |
| | CSS | | |
| | tox | | |
| | (nM) | | |
| T_{dist, L, peripheral} | Ligand | 30 | The distribution rate of ligand from the central |
| | Tdist | | compartment to the peripheral compartment. |
| | peripheral | | |
| | (hr) | | |
| T_{dist, L, disease} | Ligand | 30 | The distribution rate of ligand from the central |
| | Tdist | | compartment to the disease compartment. |
| | disease | | |
| | (hr) | | |
| T_{dist, L, tox} | Ligand | 30 | The distribution rate of ligand from the central |
| | Tdist | | compartment to the tox compartment. |
| | tox | | |
| | (hr) | | |
| T_{dist, sR, peripheral} | Soluble | 30 | The distribution rate of soluble receptor from the |
| | receptor | | central compartment to the peripheral compartment. |
| | Tdist | | |
| | peripheral | | |
| | (hr) | | |
| T_{dist, sR, disease} | Soluble | 30 | The distribution rate of soluble receptor from the |
| | receptor | | central compartment to the disease compartment. |
| | Tdist | | |
| | disease | | |
| | (hr) | | |
| T_{dist, sR, tox} | Soluble | 30 | The distribution rate of soluble receptor from the |
| | receptor | | central compartment to the tox compartment. |
| | Tdist | | |
| | tox | | |
| | (hr) | | |
| Density_{cells, central} | Cell | 1000000 | Density of receptor-bearing cells in the central |
| | density | | compartment. |
| | central | | |
| | (#/mL) | | |
| Density_{cells, peripheral} | Cell | 1000000 | Density of receptor-bearing cells in the peripheral |
| | density | | compartment. |
| | peripheral | | |
| | (#/mL) | | |
| Density_{cells, disease} | Cell | 1000000 | Density of receptor-bearing cells in the disease |
| | density | | compartment. |
| | disease | | |
| | (#/mL) | | |
| Density_{cells, tox} | Cell | 1000000 | Density of receptor-bearing cells in the tox |
| | density | | compartment. |
| | tox | | |
| | (#/mL) | | |
| Scale_{t1/2, R, D:R, central} | Drug: | 1 | A multiplicative factor for receptor turnover half-life |
| | Receptor | | when drug is bound to it in the central compartment. |
| | T ½ | | Can be used to model effects the drug has on the |
| | scale | | internalization rate of this target. |
| | central | | |
| Scale_{t1/2, R, D:R, peripheral} | Drug: | 1 | A multiplicative factor for receptor turnover half-life |
| | Receptor | | when drug is bound to it in the peripheral |
| | T ½ | | compartment. Can be used to model effects the drug |
| | scale | | has on the internalization rate of this target. |
| | peripheral | | |
| Scale_{t1/2, R, D:R, disease} | Drug: | 1 | A multiplicative factor for receptor turnover half-life |
| | Receptor | | when drug is bound to it in the disease compartment. |
| | T ½ | | Can be used to model effects the drug has on the |
| | scale | | internalization rate of this target. |
| | disease | | |
| Scale_{t1/2, R, D:R, tox} | Drug: | 1 | A multiplicative factor for receptor turnover half-life |
| | Receptor | | when drug is bound to it in the tox compartment. |
| | T ½ | | Can be used to model effects the drug has on the |
| | scale | | internalization rate of this target. |
| | tox | | |
| Scale_{KD, R, central} | Drug: | 1 | A multiplicative factor for the equilibrium |
| | Target | | dissociation constant between drug and receptor in |
| | Kd | | the central compartment. Can be used to model drugs |
| | scale | | whose target affinity is affected by the compartment. |
| | central | | |
| Scale_{KD, R, peripheral} | Drug: | 1 | A multiplicative factor for the equilibrium |
| | Target | | dissociation constant between drug and receptor in |
| | Kd | | the peripheral compartment. Can be used to model |
| | scale | | drugs whose target affinity is affected by the |
| | peripheral | | compartment. |
| Scale_{KD, R, disease} | Drug: | 1 | A multiplicative factor for the equilibrium |
| | Target | | dissociation constant between drug and receptor in |
| | Kd | | the disease compartment. Can be used to model |
| | scale | | drugs whose target affinity is affected by the |
| | disease | | compartment. |
| Scale_{KD, R, tox} | Drug: | 1 | A multiplicative factor for the equilibrium |
| | Target | | dissociation constant between drug and receptor in |
| | Kd | | the tox compartment. Can be used to model drugs |
| | scale | | whose target affinity is affected by the compartment. |
| | tox | | |
| Scale_{t1/2, central} | Soluble | 1 | A multiplicative factor for the drug elimination half- |
| | Drug | | life in the central compartment. Can be used to |
| | T ½ | | model drugs which have a different half-life in some |
| | scale | | compartments. |
| | central | | |
| Scale_{t1/2, peripheral} | Soluble | 1 | A multiplicative factor for the drug elimination half- |
| | Drug | | life in the peripheral compartment. Can be used to |
| | T ½ | | model drugs which have a different half-life in some |
| | scale | | compartments. |
| | peripheral | | |
| Scale_{t1/2, disease} | Soluble | 1 | A multiplicative factor for the drug elimination half- |
| | Drug | | life in the disease compartment. Can be used to |
| | T ½ | | model drugs which have a different half-life in some |
| | scale | | compartments. |
| | disease | | |
| Scale_{t1/2, tox} | Soluble | 1 | A multiplicative factor for the drug elimination half- |
| | Drug | | life in the tox compartment. Can be used to model |
| | T ½ | | drugs which have a different half-life in some |
| | scale | | compartments. |
| | tox | | |

TABLE 35
Example target criteria (e.g., for the therapeutic drug candidate
and the target for the therapeutic purpose) of the example monospecific
anti-receptor (4-compartment) model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free target |
| Engagement | | | due to drug binding. |
| | | | Measured as a percent of untreated. |
| Activation | 90 | percent | Percent of receptor occupied |
| | | | by ligand or drug. |
| | | | Measured as a percent of total. |
| | | | This is typically used |
| | | | for agonists. |

TABLE 36
Example output plots of the example monospecific anti-
receptor (4-compartment) model in the EFA system.

| Output Plot | Description |
|---|---|
| Criterion | The value of the selected criterion |
| vs scan | for each value of the 1D scan |
| parameter | parameter simulated. The desired |
| | target value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the |
| time course | selected criterion's time value. |
| Free drug | A time course plot of the |
| | concentration of drug not |
| | bound to anything. |
| Total drug | A time course plot of the concentration |
| | of soluble drug. This is |
| | any form of the drug not bound to |
| | any membrane proteins. This |
| | may include drug bound to one or |
| | more soluble proteins such as |
| | ligands or soluble receptors. |

In some implementations, the example monospecific anti-receptor with avidity (4-compartment) model includes a four-compartment model of avid bivalent antibody binding to membrane receptor. A biologic that binds to a cell surface receptor target and either (1) acts as a competitive inhibitor by blocking the cognate-ligand from binding to its receptor or (2) acts as a receptor agonist. The molecule can be monovalent or bivalent with avidity. This is a four-compartment model with a central, peripheral, disease and tox compartments. There is an option for +/− soluble receptor. FIG. 16A shows a schematic diagram illustrating a biotherapeutic scenario of the example monospecific anti-receptor with avidity (4-compartment) model, according to some embodiments. Example parameters of the example monospecific anti-receptor with avidity (4-compartment) model in the EFA system are shown in Table 37. Example target criteria (e.g., for the therapeutic drug candidate and the target for the therapeutic purpose) of the example monospecific anti-receptor with avidity (4-compartment) model in the EFA system are shown in Table 38. Example output plots of the example monospecific anti-receptor with avidity (4-compartment) model in the EFA system are shown in Table 39.

TABLE 37
Example parameters of the example monospecific anti-receptor
with avidity (4-compartment) model in the EFA system.

| Symbol | Name | Default | Definition |
|---|---|---|---|
| τ | Interval | 14 | The interval between doses of the drug. |
| D | Dose | 100 | The amount of each dose. |
| | (mg) | | |
| K_{D, R} | Drug: | 0.1 | The affinity of the drug for receptor as characterized |
| | Target | | by the equilibrium dissociation constant. This is the |
| | KD | | monovalent affinity (i.e. the affinity of single |
| | (nM) | | binding). |
| N_{doses} | Number of | 7 | The number of doses to be given. |
| | doses | | |
| MW | Molecular | 150000 | The molecular weight of the drug. This is used to |
| | Weight | | convert the dose which is expressed in mass units to |
| | (Da) | | a molar dose. |
| t_{1/2} | Biologic | 28 | The first-order elimination rate of the drug. |
| | first | | |
| | order | | |
| | T ½ | | |
| | (days) | | |
| t_{1/2, a} | SC | 2.5 | The first-order absorption rate of the drug from the |
| | absorption | | subcutaneous compartment into the central |
| | T ½ | | compartment. |
| | (days) | | |
| BW | Body | 70 | The body weight of the subject. This is used to |
| | Weight | | convert the dose when expressed in units of mg/kg. |
| | (kg) | | |
| V | Volume | 2.5 | The volume of the central compartment which is |
| | central | | typically the plasma of the peripheral blood. |
| | (L) | | |
| Vₚₑᵣᵢₚₕₑᵣₐₗ | Volume | 12.8 | The volume of the non-blood fluids that the antibody |
| | peripheral | | can distribute to. |
| | (L) | | |
| V_{disease} | Volume | 0.1 | The volume of the disease compartment interstitial |
| | disease | | fluid. |
| | (L) | | |
| Vₜₒₓ | Volume | 0.1 | The volume of the tox compartment interstitial fluid. |
| | tox | | |
| | (L) | | |
| T_{dist, peripheral} | Drug | 30 | The distribution rate of drug from the central |
| | Tdist | | compartment to the peripheral compartment. |
| | peripheral | | |
| | (hr) | | |
| T_{dist, disease} | Drug | 30 | The distribution rate of drug from the central |
| | Tdist | | compartment to the disease compartment. |
| | disease | | |
| | (hr) | | |
| T_{dist, tox} | Drug | 30 | The distribution rate of drug from the central |
| | Tdist | | compartment to the tox compartment. |
| | tox | | |
| | (hr) | | |
| P_{dist, peripheral} | Drug | 0.190625 | The steady-state ratio of soluble drug in the |
| | partition | | peripheral compartment to soluble drug in the central |
| | coefficient | | compartment. |
| | peripheral | | |
| P_{dist, disease} | Drug | 0.3 | The steady-state ratio of soluble drug in the disease |
| | partition | | compartment to soluble drug in the central |
| | coefficient | | compartment. |
| | disease | | |
| P_{dist, tox} | Drug | 0.3 | The steady-state ratio of soluble drug in the tox |
| | partition | | compartment to soluble drug in the central |
| | coefficient | | compartment. |
| | tox | | |
| Valency | Effective | 2 | The number of receptor molecules that a molecule of |
| | Valency | | drug can bind. This may be 1 or 2. |
| χ° | Avidity | 1 | The chi factor of a standard assay. It is the observed |
| | chi | | chi factor of an assay with with 1e6 cells/mL with a |
| | factor | | cell diameter of 10 um. |
| t_{1/2, L} | Ligand | 0.5 | The first-order elimination rate of ligand from the |
| | T ½ | | central compartment. |
| | (hr) | | |
| t_{1/2, R} | Receptor | 1 | The first-order turnover rate of receptor in all |
| | T ½ | | compartments. |
| | (hr) | | |
| t_{1/2, sR} | Soluble | 0.5 | The first-order elimination rate of soluble receptor |
| | receptor | | from the central compartment. |
| | T ½ | | |
| | (hr) | | |
| K_{D, L:R} | L:R | 1 | The binding affinity of ligand for receptor as |
| | Affinity | | characterized by the equilibrium dissociation |
| | (nM) | | constant. |
| C_{SS, L, central} | Ligand | 0.05 | The steady-state concentration of free ligand in the |
| | CSS | | central compartment in the absence of drug. Ligand |
| | central | | bound to receptor is not included in this quantity. |
| | (nM) | | |
| C_{SS, L, peripheral} | Ligand | 0.05 | The steady-state concentration of free ligand in the |
| | CSS | | peripheral compartment in the absence of drug. |
| | peripheral | | Ligand bound to receptor is not included in this |
| | (nM) | | quantity. |
| C_{SS, L, disease} | Ligand | 0.05 | The steady-state concentration of free ligand in the |
| | CSS | | disease compartment in the absence of drug. Ligand |
| | disease | | bound to receptor is not included in this quantity. |
| | (nM) | | |
| C_{SS, L, tox} | Ligand | 0.05 | The steady-state concentration of free ligand in the |
| | CSS | | tox compartment in the absence of drug. Ligand |
| | tox | | bound to receptor is not included in this quantity. |
| | (nM) | | |
| C_{SS, R, central} | Receptor | 10000 | The steady-state concentration of receptor in the |
| | CSS | | central compartment in the absence of drug. |
| | central | | Receptor bound to ligand is included in this quantity, |
| | (#/cell) | | but soluble receptor is not. |
| C_{SS, R, peripheral} | Receptor | 10000 | The steady-state concentration of receptor in the |
| | CSS | | peripheral compartment in the absence of drug. |
| | peripheral | | Receptor bound to ligand is included in this quantity, |
| | (#/cell) | | but soluble receptor is not. |
| C_{SS, R, disease} | Receptor | 10000 | The steady-state concentration of receptor in the |
| | CSS | | disease compartment in the absence of drug. |
| | disease | | Receptor bound to ligand is included in this quantity, |
| | (#/cell) | | but soluble receptor is not. |
| C_{SS, R, tox} | Receptor | 10000 | The steady-state concentration of receptor in the tox |
| | CSS | | compartment in the absence of drug. Receptor bound |
| | tox | | to ligand is included in this quantity, but soluble |
| | (#/cell) | | receptor is not. |
| C_{SS, sR, central} | Soluble | 0 | The steady-state concentration of soluble receptor in |
| | receptor | | the central compartment in the absence of drug. |
| | CSS | | |
| | central | | |
| | (nM) | | |
| C_{SS, sR, peripheral} | Soluble | 0 | The steady-state concentration of soluble receptor in |
| | receptor | | the peripheral compartment in the absence of drug. |
| | CSS | | |
| | peripheral | | |
| | (nM) | | |
| C_{SS, sR, disease} | Soluble | 0 | The steady-state concentration of soluble receptor in |
| | receptor | | the disease compartment in the absence of drug. |
| | CSS | | |
| | disease | | |
| | (nM) | | |
| C_{SS, sR, tox} | Soluble | 0 | The steady-state concentration of soluble receptor in |
| | receptor | | the tox compartment in the absence of drug. |
| | CSS | | |
| | tox | | |
| | (nM) | | |
| T_{dist, L, peripheral} | Ligand | 30 | The distribution rate of ligand from the central |
| | Tdist | | compartment to the peripheral compartment. |
| | peripheral | | |
| | (hr) | | |
| T_{dist, L, disease} | Ligand | 30 | The distribution rate of ligand from the central |
| | Tdist | | compartment to the disease compartment. |
| | disease | | |
| | (hr) | | |
| T_{dist, L, tox} | Ligand | 30 | The distribution rate of ligand from the central |
| | Tdist | | compartment to the tox compartment. |
| | tox | | |
| | (hr) | | |
| T_{dist, sR, peripheral} | Soluble | 30 | The distribution rate of soluble receptor from the |
| | receptor | | central compartment to the peripheral compartment. |
| | Tdist | | |
| | peripheral | | |
| | (hr) | | |
| T_{dist, sR, disease} | Soluble | 30 | The distribution rate of soluble receptor from the |
| | receptor | | central compartment to the disease compartment. |
| | Tdist | | |
| | disease | | |
| | (hr) | | |
| T_{dist, sR, tox} | Soluble | 30 | The distribution rate of soluble receptor from the |
| | receptor | | central compartment to the tox compartment. |
| | Tdist | | |
| | tox | | |
| | (hr) | | |
| Diam_{cell} | Cell | 10 | Diameter of receptor-bearing cells. |
| | diameter | | |
| | (um) | | |
| Density_{cells, central} | Cell | 1000000 | Density of receptor-bearing cells in the central |
| | density | | compartment. |
| | central | | |
| | (#/mL) | | |
| Density_{cells, peripheral} | Cell | 1000000 | Density of receptor-bearing cells in the peripheral |
| | density | | compartment. |
| | peripheral | | |
| | (#/mL) | | |
| Density_{cells, disease} | Cell | 1000000 | Density of receptor-bearing cells in the disease |
| | density | | compartment. |
| | disease | | |
| | (#/mL) | | |
| Density_{cells, tox} | Cell | 1000000 | Density of receptor-bearing cells in the tox |
| | density | | compartment. |
| | tox | | |
| | (#/mL) | | |
| Scale_{t1/2, R, D:R, central} | Drug: | 1 | A multiplicative factor for receptor turnover half-life |
| | Receptor | | when drug is bound to it in the central compartment. |
| | T ½ | | Can be used to model effects the drug has on the |
| | scale | | internalization rate of this target. |
| | central | | |
| Scale_{t1/2, R, D:R, peripheral} | Drug: | 1 | A multiplicative factor for receptor turnover half-life |
| | Receptor | | when drug is bound to it in the peripheral |
| | T ½ | | compartment. Can be used to model effects the drug |
| | scale | | has on the internalization rate of this target. |
| | peripheral | | |
| Scale_{t1/2, R, D:R, disease} | Drug: | 1 | A multiplicative factor for receptor turnover half-life |
| | Receptor | | when drug is bound to it in the disease compartment. |
| | T ½ | | Can be used to model effects the drug has on the |
| | scale | | internalization rate of this target. |
| | disease | | |
| Scale_{t1/2, R, D:R, tox} | Drug: | 1 | A multiplicative factor for receptor turnover half-life |
| | Receptor | | when drug is bound to it in the tox compartment. |
| | T ½ | | Can be used to model effects the drug has on the |
| | scale | | internalization rate of this target. |
| | tox | | |
| Scale_{KD, R, central} | Drug: | 1 | A multiplicative factor for the equilibrium |
| | Target | | dissociation constant between drug and receptor in |
| | Kd | | the central compartment. Can be used to model drugs |
| | scale | | whose target affinity is affected by the compartment. |
| | central | | |
| Scale_{KD, R, peripheral} | Drug: | 1 | A multiplicative factor for the equilibrium |
| | Target | | dissociation constant between drug and receptor in |
| | Kd | | the peripheral compartment. Can be used to model |
| | scale | | drugs whose target affinity is affected by the |
| | peripheral | | compartment. |
| Scale_{KD, R, disease} | Drug: | 1 | A multiplicative factor for the equilibrium |
| | Target | | dissociation constant between drug and receptor in |
| | Kd | | the disease compartment. Can be used to model |
| | scale | | drugs whose target affinity is affected by the |
| | disease | | compartment. |
| Scale_{KD, R, tox} | Drug: | 1 | A multiplicative factor for the equilibrium |
| | Target | | dissociation constant between drug and receptor in |
| | Kd | | the tox compartment. Can be used to model drugs |
| | scale | | whose target affinity is affected by the compartment. |
| | tox | | |
| Scale_{t1/2, central} | Soluble | 1 | A multiplicative factor for the drug elimination half- |
| | Drug | | life in the central compartment. Can be used to |
| | T ½ | | model drugs which have a different half-life in some |
| | scale | | compartments. |
| | central | | |
| Scale_{t1/2, peripheral} | Soluble | 1 | A multiplicative factor for the drug elimination half- |
| | Drug | | life in the peripheral compartment. Can be used to |
| | T ½ | | model drugs which have a different half-life in some |
| | scale | | compartments. |
| | peripheral | | |
| Scale_{t1/2, disease} | Soluble | 1 | A multiplicative factor for the drug elimination half- |
| | Drug | | life in the disease compartment. Can be used to |
| | T ½ | | model drugs which have a different half-life in some |
| | scale | | compartments. |
| | disease | | |
| Scale_{t1/2, tox} | Soluble | 1 | A multiplicative factor for the drug elimination half- |
| | Drug | | life in the tox compartment. Can be used to model |
| | T ½ | | drugs which have a different half-life in some |
| | scale | | compartments. |
| | tox | | |

TABLE 38
Example target criteria (e.g., for the therapeutic
drug candidate and the target for the therapeutic
purpose) of the example monospecific anti-receptor
with avidity (4-compartment) model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free target |
| Engagement | | | due to drug binding. |
| | | | Measured as a percent of untreated. |
| Activation | 90 | percent | Percent of receptor occupied by |
| | | | ligand or drug. |
| | | | Measured as a percent of total. |
| | | | This is typically used |
| | | | for agonists. |

TABLE 39
Example output plots of the example monospecific anti-receptor
with avidity (4-compartment) model in the EFA system.

| Output Plot | Description |
|---|---|
| Criterion | The value of the selected criterion for |
| vs scan | each value of the 1D scan |
| parameter | parameter simulated. The desired |
| | target value is plotted as a |
| | horizontal dashed line. |
| Criterion | The value of the selected criterion |
| heatmap | for pair of 2D scan parameters. The range |
| | of the heatmap color bar is fixed for each |
| | criterion. |
| Criterion | A time course plot of the selected |
| time course | criterion's time value. |
| Free drug | A time course plot of the concentration |
| | of drug not bound to anything. |
| Total drug | A time course plot of the concentration |
| | of soluble drug. This is any form of the drug |
| | not bound to any membrane proteins. This |
| | may include drug bound to one or more |
| | soluble proteins such as |
| | ligands or soluble receptors. |

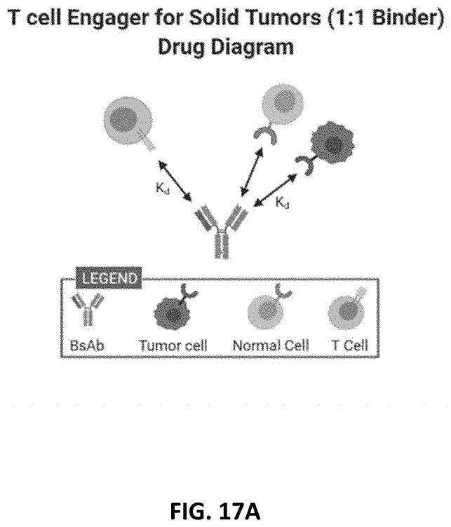

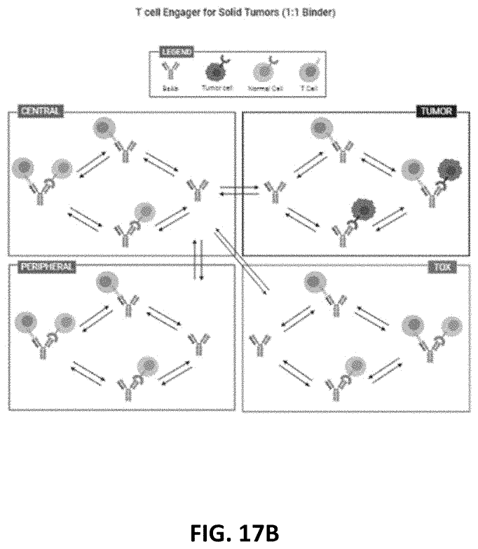

In some implementations, the example T cell engager for solid tumors model includes a four-compartment model of a biotherapeutic that crosslinks T cell receptor (TCR) on T cells to a tumor associated antigen (TAA) on tumor cells and normal cells. A T cell engager (TCE) biologic for solid tumors. This is a bispecific biologic molecule that is monovalent for T cell receptor (TCR) on T cells, and mono- or bivalent for a tumor associated antigen (TAA). The model describes the formation of trimers consisting of transcell complexes consisting of TCR—Drug—TAA or TCR—Drug—TAA₂. The model has four compartments representing the peripheral blood (Central), solid tumor (Tumor), off tumor toxicity tissue (Tox), and the rest of the body (Peripheral). There are T-cells and TAA expressing cells in all four compartments. The model output is trimer per T cell in tumor and non-tumor tissues as well as the ratio of trimer in the tumor and tox compartments. FIGS. 17A-17B show schematic diagrams illustrating a biotherapeutic scenario of the example T cell engager for solid tumors model, according to some embodiments. Example parameters of the example T cell engager for solid tumors model in the EFA system are shown in Table 40. Example target criteria (e.g., for the therapeutic drug candidate and the target for the therapeutic purpose) of the example T cell engager for solid tumors model in the EFA system are shown in Table 41. Example output plots of the example T cell engager for solid tumors model in the EFA system are shown in Table 42.

TABLE 40
Example parameters of the example T cell engager for solid tumors model in the EFA system.

| Symbol | Name | Default | Definition |
|---|---|---|---|
| τ | Interval | 14 | The interval between doses of the drug. |
| D | Dose | 1 | The amount of drug given per dose. |
| | (mg) | | |
| N_{doses} | Number | 7 | The number of repeated doses to give. |
| | of doses | | |
| MW | Molecular | 150000 | The molecular weight of the drug. This is used to |
| | weight | | convert the dose which is expressed in mass units to |
| | (Da) | | a molar dose. |
| BW | Body | 70 | The body weight of the subject. |
| | weight | | |
| | (kg) | | |
| t_{1/2, a} | SC | 2.5 | The rate of absorption of a subcutaneously |
| | absorption T | | administered drug. |
| | ½ | | |
| | (days) | | |
| t_{1/2} | Biologic | 14 | The rate of elimination of drug. |
| | first | | |
| | order T | | |
| | ½ | | |
| | (days) | | |
| K_{D, TAA} | Drug: T | 1 | The affinity of the drug for the tumor associated |
| | AA KD | | antigen (TAA) target as measured by the equilibrium |
| | (nM) | | dissociation constant. This is the monovalent affinity |
| | | | (i.e. the affinity of a single binding site for TAA). |
| K_{D, TCR} | Drug: T | 100 | The affinity of the drug for the TCR as measured by |
| | CR KD | | the equilibrium dissociation constant. This is the |
| | (nM) | | monovalent affinity (i.e. the affinity of a single |
| | | | binding site for TCR). |
| V | Volume | 2.5 | The volume of the central compartment which is |
| | central | | typically the plasma of the peripheral blood. |
| | (L) | | |
| Vₚₑᵣᵢₚₕₑᵣₐₗ | Volume | 12.8 | The volume of the non blood fluids that the antibody |
| | peripheral | | can distribute to. |
| | (L) | | |
| Vₜᵤₘₒᵣ | Volume | 50.1 | The volume of the disease compartment interstitial |
| | tumor | | fluid. |
| | (L) | | |
| Vₜₒₓ | Volume | 0.1 | The volume of the tox compartment interstitial fluid. |
| | tox | | |
| | (L) | | |
| T_{dist, peripheral} | Drug | 30 | The rate at which drug equilibrates with the |
| | Tdist | | peripheral compartment. |
| | peripheral | | |
| | (hr) | | |
| T_{dist, tumor} | Drug | 30 | The rate at which drug equilibrates with the tumor |
| | Tdist | | compartment. |
| | tumor | | |
| | (hr) | | |
| T_{dist, tox} | Drug | 30 | The rate at which drug equilibrates with the tox |
| | Tdist | | compartment. |
| | tox | | |
| | (hr) | | |
| P_{dist, peripheral} | Drug | 0.190625 | The steady state ratio of free drug in the peripheral |
| | Pdist | | compartment compared to the central compartment. |
| | peripheral | | Note that target binding can influence this ratio so |
| | | | this is really the ratio in the absence of any target |
| | | | binding. |
| P_{dist, tumor} | Drug | 0.3 | The steady state ratio of free drug in the tumor |
| | Pdist | | compartment compared to the central compartment. |
| | tumor | | Note that target binding can influence this ratio so |
| | | | this is really the ratio in the absence of any target |
| | | | binding. |
| P_{dist, tox} | Drug | 0.3 | The steady state ratio of free drug in the tox |
| | Pdist | | compartment compared to the central compartment. |
| | tox | | Note that target binding can influence this ratio so |
| | | | this is really the ratio in the absence of any target |
| | | | binding. |
| Valency_{TAA} | Effective | 1 | The number of TAA molecules that the drug can |
| | valency | | bind. This may be 1 or 2. |
| | to TAA | | |
| χ°_{cis} | Cis | 30000 | The chi factor of a standard assay for binding to |
| | avidity | | TAA when already bound to TAA. It is the observed |
| | chi | | chi factor of an assay with with 1e6 cells/mL with a |
| | factor | | cell diameter of 10 um. |
| χ°ₜᵣₐₙₛ | Transavidity | 30000 | The chi factor of a standard assay for binding to |
| | chi | | TCR when already bound to TAA or binding to |
| | factor | | TAA when already bound to TCR but not TAA. It is |
| | | | the observed chi factor of an assay with with 1e6 |
| | | | cells/mL with a cell diameter of 10 um. |
| t_{1/2, TAA} | TAA | 1 | The turnover rate of free TAA on tumor and non- |
| | first | | tumor cells. |
| | order T | | |
| | ½ | | |
| | (hr) | | |
| t_{1/2, TCR} | TCR | 20 | The turnover rate of free TCR on effector T cells. |
| | first | | |
| | order T | | |
| | ½ | | |
| | (hr) | | |
| t_{1/2, sTAA} | Shed | 0.5 | The half-life of free soluble TAA. |
| | TAA T | | |
| | ½ | | |
| | (hr) | | |
| t_{1/2, sTCR} | Shed | 0.5 | The half-life of free soluble TCR. |
| | TCR T | | |
| | ½ | | |
| | (hr) | | |
| C_{SS, TAA, central} | TAA | 10000 | Density of tumor associated antigens (TAA) per |
| | density | | non-tumor cell in the central compartment. |
| | central | | |
| | (#/cell) | | |
| C_{SS, TAA, peripheral} | TAA | 10000 | Density of tumor associated antigens (TAA) per |
| | density | | non-tumor cell in the peripheral compartment. |
| | peripheral | | |
| | (#/cell) | | |
| C_{SS, TAA, tumor} | TAA | 10000 | Density of tumor associated antigens (TAA) per |
| | density | | tumor cell in the tumor compartment. |
| | tumor | | |
| | (#/cell) | | |
| C_{SS, TAA, tox} | TAA | 10000 | Density of tumor associated antigens (TAA) per |
| | density | | non-tumor cell in the tox compartment. |
| | tox | | |
| | (#/cell) | | |
| C_{SS, TCR, central} | TCR | 62000 | Density of CD3 expressed per effector T cell in the |
| | density | | central compartment. |
| | central | | |
| | (#/cell) | | |
| C_{SS, TCR, peripheral} | TCR | 62000 | Density of CD3 expressed per effector T cell in the |
| | density | | peripheral compartment. |
| | peripheral | | |
| | (#/cell) | | |
| C_{SS, TCR, tumor} | TCR | 62000 | Density of CD3 expressed per effector T cell in the |
| | density | | tumor compartment. |
| | tumor | | |
| | (#/cell) | | |
| C_{SS, TCR, tox} | TCR | 62000 | Density of CD3 expressed per effector T cell in the |
| | density | | tox compartment. |
| | tox | | |
| | (#/cell) | | |
| C_{SS, sTAA, central} | Shed | 0 | The steady state concentration of soluble TAA in the |
| | TAA | | absence of drug in the central compartment. |
| | density | | |
| | central | | |
| | (nM) | | |
| C_{SS, sTAA, peripheral} | Shed | 0 | The steady state concentration of soluble TAA in the |
| | TAA | | absence of drug in the peripheral compartment. |
| | density | | |
| | peripheal | | |
| | (nM) | | |
| C_{SS, sTAA, tumor} | Shed | 0 | The steady state concentration of soluble TAA in the |
| | TAA | | absence of drug in the tumor compartment. |
| | density | | |
| | tumor | | |
| | (nM) | | |
| C_{SS, sTAA, tox} | Shed | 0 | The steady state concentration of soluble TAA in the |
| | TAA | | absence of drug in the tox compartment. |
| | density | | |
| | tox | | |
| | (nM) | | |
| C_{SS, sTCR, central} | Shed | 0 | The steady state concentration of soluble TCR in the |
| | TCR | | absence of drug in the central compartment. |
| | density | | |
| | central | | |
| | (nM) | | |
| C_{SS, sTCR, peripheral} | Shed | 0 | The steady state concentration of soluble TCR in the |
| | TCR | | absence of drug in the peripheral compartment. |
| | density | | |
| | peripheral | | |
| | (nM) | | |
| C_{SS, sTCR, tumor} | Shed | 0 | The steady state concentration of soluble TCR in the |
| | TCR | | absence of drug in the tumor compartment. |
| | density | | |
| | tumor | | |
| | (nM) | | |
| C_{SS, sTCR, tox} | Shed | 0 | The steady state concentration of soluble TCR in the |
| | TCR | | absence of drug in the tox compartment. |
| | density | | |
| | tox | | |
| | (nM) | | |
| T_{dist, sTAA, peripheral} | Shed | 30 | The time for TAA to distribute from the central |
| | TAA | | compartment to the peripheral compartment. |
| | Tdist | | |
| | peripheral | | |
| | (hr) | | |
| T_{dist, sTAA, tumor} | Shed | 30 | The time for TAA to distribute from the central |
| | TAA | | compartment to the tumor compartment. |
| | Tdist | | |
| | tumor | | |
| | (hr) | | |
| T_{dist, sTAA, tox} | Shed | 30 | The time for TAA to distribute from the central |
| | TAA | | compartment to the tox compartment. |
| | Tdist | | |
| | tox | | |
| | (hr) | | |
| T_{dist, sTCR, peripheral} | Shed | 30 | The time for TCR to distribute from the central |
| | TCR | | compartment to the peripheral compartment. |
| | Tdist | | |
| | peripheral | | |
| | (hr) | | |
| T_{dist, sTCR, tumor} | Shed | 30 | The time for TCR to distribute from the central |
| | TCR | | compartment to the tumor compartment. |
| | Tdist | | |
| | tumor | | |
| | (hr) | | |
| T_{dist, sTCR, tox} | Shed | 30 | The time for TCR to distribute from the central |
| | TCR | | compartment to the tox compartment. |
| | Tdist | | |
| | tox | | |
| | (hr) | | |
| Diam_{target-cell} | Target | 15 | Diameter of TAA-bearing cells. |
| | cell | | |
| | diameter | | |
| | (um) | | |
| Diam_{T-cell} | T cell | 7 | Diameter of TCR-bearing cells. |
| | diameter | | |
| | (um) | | |
| Density_{target-cell, central} | Target | 0 | The density of tumor associated antigen (TAA) |
| | cell | | positive non-tumor cells in the central compartment. |
| | density | | |
| | central | | |
| | (mL) | | |
| Density_{target-cell, peripheral} | Target | 0 | The density of tumor associated antigen (TAA) |
| | cell | | positive non-tumor cells in the central compartment. |
| | density | | |
| | peripheral | | |
| | (mL) | | |
| Density_{target-cell, tumor} | Tumor | 100000000 | The density of tumor associated antigen (TAA) |
| | cell | | positive tumor cells in the tumor compartment. |
| | density | | |
| | (mL) | | |
| Density_{target-cell, tox} | Target | 0 | The density of tumor associated antigen (TAA) |
| | cell | | positive non-tumor cells in the tox compartment. |
| | density | | |
| | tox | | |
| | (mL) | | |
| Density_{T-cell, central} | T cell | 1170000 | The density of T cells in the central (plasma) |
| | density | | compartment. |
| | central | | |
| | (mL) | | |
| Density_{T-cell, peripheral} | T cell | 20200000 | The density of T cells in the peripheral compartment. |
| | density | | |
| | peripheral | | |
| | (mL) | | |
| Density_{T-cell, tumor} | T cell | 1000000 | The density of T cells in the tumor compartment. |
| | density | | |
| | tumor | | |
| | (mL) | | |
| Density_{T-cell, tox} | T cell | 1000000 | The density of T cells in the tox compartment. |
| | density | | |
| | tox | | |
| | (mL) | | |
| Scale_{t1/2, central} | Soluble | 1 | A factor that gets multiplied to the half-life in the |
| | Drug T | | central compartment. So the half-life will become: |
| | ½ | | Biologic first-order T ½ * Soluble Drug T ½ scale |
| | scale | | central |
| | central | | |
| Scale_{t1/2, peripheral} | Soluble | 1 | A factor that gets multiplied to the half-life in the |
| | Drug T | | peripheral compartment. So the half-life will |
| | ½ | | become: Biologic first-order T ½ * Soluble Drug T |
| | scale | | ½ scale peripheral |
| | peripheral | | |
| Scale_{t1/2, tumor} | Soluble | 1 | A factor that gets multiplied to the half-life in the |
| | Drug T | | tumor compartment. So the half-life will become: |
| | ½ | | Biologic first-order T ½ * Soluble Drug T ½ scale |
| | scale | | tumor |
| | tumor | | |
| Scale_{t1/2, tox} | Soluble | 1 | A factor that gets multiplied to the half-life in the tox |
| | Drug T | | compartment. So the half-life will become: Biologic |
| | ½ | | first-order T ½ * Soluble Drug T ½ scale tox |
| | scale | | |
| | tox | | |
| Scale_{KD, TAA, central} | Drug: T | 1 | A factor that gets multiplied to the Kd of Drug |
| | AA KD | | binding to TAA in the central compartment. So the |
| | scale | | Kd will become: Drug: TAA KD * Drug: TAA KD |
| | central | | scale central |
| Scale_{KD, TAA, peripheral} | Drug: T | 1 | A factor that gets multiplied to the Kd of Drug |
| | AA KD | | binding to TAA in the peripheral compartment. So |
| | scale | | the Kd will become: Drug: TAA KD * Drug: TAA |
| | peripheral | | KD scale peripheral |
| Scale_{KD, TAA, tumor} | Drug: T | 1 | A factor that gets multiplied to the Kd of Drug |
| | AA KD | | binding to TAA in the tumor compartment. So the |
| | scale | | Kd will become: Drug: TAA KD * Drug: TAA KD |
| | tumor | | scale tumor |
| Scale_{KD, TAA, tox} | Drug: T | 1 | A factor that gets multiplied to the Kd of Drug |
| | AA KD | | binding to TAA in the tox compartment. So the Kd |
| | scale | | will become: Drug: TAA KD * Drug: TAA KD scale |
| | tox | | tox |
| Scale_{KD, TCR, central} | Drug: T | 1 | A factor that gets multiplied to the Kd of Drug |
| | CR KD | | binding to TCR in the central compartment. So the |
| | scale | | Kd will become: Drug: TCR KD * Drug: TCR KD |
| | central | | scale central |
| Scale_{KD, TCR, peripheral} | Drug: T | 1 | A factor that gets multiplied to the Kd of Drug |
| | CR KD | | binding to TCR in the peripheral compartment. So |
| | scale | | the Kd will become: Drug: TCR KD * Drug: TCR KD |
| | peripheral | | scale peripheral |
| Scale_{KD, TCR, tumor} | Drug: T | 1 | A factor that gets multiplied to the Kd of Drug |
| | CR KD | | binding to TCR in the tumor compartment. So the |
| | scale | | Kd will become: Drug: TCR KD * Drug: TCR KD |
| | tumor | | scale tumor |
| Scale_{KD, TCR, tox} | Drug: T | 1 | A factor that gets multiplied to the Kd of Drug |
| | CR KD | | binding to TCR in the tox compartment. So the Kd |
| | scale | | will become: Drug: TCR KD * Drug: TCR KD scale |
| | tox | | tox |
| Scale_{t1/2, TAA, D: TAA, central} | Drug: T | 1 | A factor that gets multiplied to the half-life of |
| | AA T | | drug: TAA complex in the central compartment. So |
| | ½ | | the half-life will become: Drug: TAA T ½ * |
| | scale | | Drug: TAA T ½ scale central |
| | central | | |
| Scale_{t1/2, TAA, D: TAA, peripheral} | Drug: T | 1 | A factor that gets multiplied to the half-life of |
| | AA T | | drug: TAA complex in the peripheral compartment. |
| | ½ | | So the half-life will become: Drug: TAA T ½ * |
| | scale | | Drug: TAA T ½ scale peripheral |
| | peripheral | | |
| Scale_{t1/2, TAA, D: TAA, tumor} | Drug: T | 1 | A factor that gets multiplied to the half-life of |
| | AA T | | drug: TAA complex in the tumor compartment. So |
| | ½ | | the half-life will become: Drug: TAA T ½ * |
| | scale | | Drug: TAA T ½ scale tumor |
| | tumor | | |
| Scale_{t1/2, TAA, D: TAA, tox} | Drug: T | 1 | A factor that gets multiplied to the half-life of |
| | AA T | | drug: TAA complex in the tox compartment. So the |
| | ½ | | half-life will become: Drug: TAA T ½ * Drug: TAA |
| | scale | | T ½ scale tox |
| | tox | | |
| Scale_{t1/2, TCR, D: TAA, central} | Drug: T | 1 | A factor that gets multiplied to the half-life of |
| | CR T | | drug: TCR complex in the central compartment. So |
| | ½ | | the half-life will become: Drug: TCR T ½ * |
| | scale | | Drug: TCR T ½ scale central |
| | central | | |
| Scale_{t1/2, TCR, D: TAA, peripheral} | Drug: T | 1 | A factor that gets multiplied to the half-life of |
| | CR T | | drug: TCR complex in the peripheral compartment. |
| | ½ | | So the half-life will become: Drug: TCR T ½ * |
| | scale | | Drug: TCR T ½ scale peripheral |
| | peripheral | | |
| Scale_{t1/2, TCR, D: TAA, tumor} | Drug: T | 1 | A factor that gets multiplied to the half-life of |
| | CR T | | drug: TCR complex in the tumor compartment. So |
| | ½ | | the half-life will become: Drug: TCR T ½ * |
| | scale | | Drug: TCR T ½ scale tumor |
| | tumor | | |
| Scale_{t1/2, TCR, D: TAA, tox} | Drug: T | 1 | A factor that gets multiplied to the half-life of |
| | CR T | | drug: TCR complex in the tox compartment. So the |
| | ½ | | half-life will become: Drug: TCR T ½ * Drug: TCR |
| | scale | | T ½ scale tox |
| | tox | | |

TABLE 41
Example target criteria (e.g., for the therapeutic drug candidate
and the target for the therapeutic purpose) of the example
T cell engager for solid tumors model in the EFA system.

| | Default | | |
|---|---|---|---|
| Criterion | Target | Units | Description |
| Inhibition | 90 | percent | Inhibition of ligand receptor |
| | | | complex. Measured as a |
| | | | percent of untreated. |
| Target | 90 | percent | Percent reduction of free |
| Engagement | | | target due to drug binding. |
| | | | Measured as a percent of untreated. |
| Activation | 90 | percent | Percent of receptor occupied by |
| | | | ligand or drug. |
| | | | Measured as a percent of |
| | | | total. This is typically used |
| | | | for agonists. |

TABLE 42
Example output plots of the example T cell engager
for solid tumors model in the EFA system.

| Output Plot | Description |
|---|---|
| Criterion | The value of the selected criterion |
| vs scan | for each value of the ID scan |
| parameter | parameter simulated. The desired |
| | target value is plotted as a |
| | horizontal dashed line. |
| Criterion | The value of the selected criterion |
| heatmap | for pair of 2D scan |
| | parameters. The range of the heatmap |
| | color bar is fixed for each |
| | criterion. |
| Criterion | A time course plot of the selected |
| time course | criterion's time value. |
| Free drug | A time course plot of the |
| | concentration of drug not bound to |
| | anything. |
| Total drug | A time course plot of the concentration |
| | of soluble drug. This is any form of the |
| | drug not bound to any membrane proteins. This |
| | may include drug bound to one or more |
| | soluble proteins such as ligands or soluble receptors. |

In some implementations, additional models can be used in the EFA system, including, for example, Antibody Drug Conjugates (ADC) with specific targeting MOA (e.g., mAb, bsAb, Activatable Antibodies, etc.), Viral or Lipid Nanoparticle (LNP) Delivery of Nucleotide, targeted LNP Delivery, Endosome Delivery and Targeted Endosome Delivery of Delivery of Nucleotide with specific Nucleotide versions (e.g. mRNA, DNA, RNAi, shRNA, etc), Therapeutic proteins (e.g., mAbs, antibody-fusion, bsAb, tsAb, peptides, etc), Small molecule drugs, including protein homeostasis/protein degraders, and irreversible binders, Cell therapies (e.g. CAR T cell, CAR NK cell, platelets, etc.), cell engagers for solid and liquid tumors including T cell engagers, NK cell engagers, Macrophage engagers, with and without Tumor growth inhibition (TGI), targeted protein degraders, cytokines, Activatable Antibodies (AA), Oncolytic Viruses, PBPK models for small molecules and large molecules, and any PK/TE therapeutic, mechanistic PK/PD, QSP, or more complex model with downstream mechanisms.

### Example Mechanisms to Construct the Example Models in the EFA System

In some implementations, the process of constructing the QSP models (e.g., the example models in the EFA system discussed above including the bispecific anti-ligand x anti-ligand model) can include using mass action kinetics with zeroth-, first-, and second-order kinetics. This process of constructing the QSP models can be implemented at a processor and/or a memory (e.g., processor 111 or memory 112 at the server 101 as discussed with respect to FIG. 1, the processor 121 or memory 122 at the client 102 as described with respect to FIG. 1, and/or the processor or memory at computing devices that are operatively or communicatively coupled to the server 101 discussed with respect to FIG. 1).

The process of constructing the QSP models includes determining drug dosage parameters, drug absorption parameters, drug distribution parameters, and drug elimination parameters.

In some implementations, the drug dosage parameters in each QSP model can include the dose amount, dose interval, number of repeated doses, and the route of administration.

### Dose Amount

The EFA system supports QSP models with flat dosing and body weight normalized dosing. Each model can have four parameters that control dose: Dose (mg), Dose (mg/kg), Body Weight (kg), and Molecular Weight (kDa). Users control the flat vs. body weight dosing by selecting the “Route” parameter

TABLE 43

| Route | Name | Total Amount | Default |
|---|---|---|---|
| SC_mg | Subcutaneous Flat | Dose (mg) | 100 mg |
| SC_mpk | Subcutaneous | Dose (mg/kg) * | 1 mg/kg * |
| | Normalized | Body Weight (kg) | 70 kg |
| IV_mg | IV Flat Dosing | Dose (mg) | 100 mg |
| IV_mpk | IV Normalized | Dose (mg/kg) * | 1 mg/kg * |
| | Dosing | Body Weight (kg) | 70 kg |

Total doses can be converted to molar quantities with the molecular weight field. The default value of 100 mg corresponds to a typical dose of a mAb administered by subcutaneous injection.

### Dose Intervals

In the EFA system, doses can be given on a fixed dose schedule. The parameter that sets the dose interval is called “interval” in most models. Values are chosen from a dropdown list, the values are shown in Table 44.

TABLE 44

| Interval | Name | Time (Days) |
|---|---|---|
| QD | Once Daily | 1 |
| TIW | Three Times Weekly | 2.33 |
| BIW | Twice Weekly | 3.5 |
| Q1W | Weekly | 7 |
| Q2W | Every two weeks | 14 |
| Q3W | Every three weeks | 21 |
| Q1M | Every Month | 28 |
| Q2M | Every Two Months | 56 |
| Q3M | Every Three Months | 84 |

### Routes of Administration

Doses may be given by various routes of administration. The default models all define a subcutaneous route of administration and an intravenous route of administration. Custom models may have additional routes which will be described in the model documentation.

TABLE 45

| Route | Name | Description |
|---|---|---|
| IV | Intravenous | Intravenous dosing is modeled as a |
| | | bolus dose into the central |
| | | compartment |
| SC | Subcutaneous | Subcutaneous dosing is extravascular |
| | | and it has an absorption |
| | | rate SC absorption T ½ (days) |
| | | which is the time for half of the |
| | | drug to be absorbed into the central |
| | | compartment. Note: for the |
| | | SC route, the bioavailability is |
| | | assumed to be 100%. To model |
| | | lower bioavailability, reduce |
| | | the dose by the appropriate fraction. |

### Drug Absorption

When a drug is dosed with the Subcutaneous route, it is absorbed into the central compartment with linear first order absorption kinetics. The SC absorption T ½ (days) parameter sets the absorption rate. This rate is converted to a first order absorption rate using:

$${k}_{a b s} = ln ( 2 ) / {T}_{1 / 2}$$

### Drug Elimination

Drugs are assumed to be eliminated by linear first order kinetics. The Biologic first order T ½ (days) parameter determines the elimination rate. This rate is converted to a first order elimination rate using:

$${k}_{el} = ln ( 2 ) / {T}_{1 / 2}$$

In multicompartment models, the same rate can be applied to all compartments. Note: other routes of elimination, such as target mediated elimination, can increase the overall drug elimination, and are independent of this mechanism.

Drug Distribution:

In multicompartment models, drug will transport from the central compartment to the peripheral compartment with 1st order mass action kinetics described by a first order rate constant kout (1/s). Transport from the peripheral compartment to the central compartment will likewise be first order mass action with rate constant kin(1/s).

Reaction Schema: D_<cpt1><--kin, kout-->D_<cpt2>

Relationships: the QSP Models can define macro parameters that describe the steady state concentration ratio between the central compartment and the tissue compartment (Pdist) and the time it takes to reach equilibrium defined as a half-life (Tdist).

$$R = \frac{ln ( 2 )}{Tdist 12}$$

$$kin = R \frac{Pdist 12}{( Pdist 12 + \frac{V 1}{V 2} )}$$

$$kout = R \frac{1}{( 1 + Pdist \times \frac{V 2}{V 1} )}$$

An example value for Tdist is 15 min for smaller proteins to 12 to 24 hrs for larger proteins like IgGs. A example value of Pdist is 0.3.

### Soluble Drug Complex Transport and Elimination

Drugs bound to shed and or soluble receptors can also transport between the central and peripheral compartment. The assumption is that they also transport with 1st order mass action kinetics but the rates may be altered compared to the free drug. Note that in the case of multivalent drugs there are potentially many complexes representing binding to one or more ligands or shed receptors. It is assumed that all of these have the same distribution kinetics and are described by the same two parameters. The default value for these parameters will be the same as the corresponding parameter for the free drug. It is possible to adjust the elimination rate for drug complexes using a multiplier. This would be used to model cases where the drug protein complex cleared faster or slower than the free drug.

Relationships: The rates of distribution, and elimination may be adjusted for complexes by setting a compartment specific multiplier

$$Tdist {12}_{complex} = Tdist 12 * kmul {t}_{{Tdist 12}_{complex}} \\ Pdist {12}_{complex} = Pdist 12 * kmul {t}_{{Pdist 12}_{complex}}$$

### Drug Bound to One or More Membrane Receptors

There are several options for the disposition of internalized receptor drug complexes. The drug may either stay bound or unbind when the receptor internalizes, or the drug may stay bound bringing one or more members of the complex when it internalizes. In the EFA models the default assumption is that for cis complexes there is an internalization reaction associated with each receptor. The rate of internalization is the same as that of the free receptor. In the internalization reaction, in some implementations, only the receptor associated with the internalized receptor is lost in the reaction. This corresponds to an assumption of unbinding to the other receptors. In some models an alternative assumption is that all members of the complex are internalized. In some models the drug dissociates and is not internalized. In some models an assumption that only strongly bound receptors are internalized where strongly bound is determined by an affinity cut off. In some models trans complexes (complexes that bridge both cells) the same set of options can be applied.

In some implementations, the process of constructing the QSP models includes determining protein synthesis parameters, protein trafficking parameters, and protein elimination parameters.

### Zeroth-Order Soluble Protein Synthesis and First-Order Clearance of Soluble Proteins

In some implementations, soluble proteins are assumed to be made with zeroth-order kinetics. This is to say that the upstream process of transcription and translation is assumed to be at steady state and constant. Soluble protein synthesis that occurs in a compartment is described by first order rate constant k_synth_<protein>_<compartment> (mol⁻¹ sec⁻¹). This does not depend on numbers of cells or any other process, although it may be unequal to reflect differences in protein levels in different compartments.

Reaction Schema: 0—ksynth_<sol>_<cpt>-><Sol>

### Clearance of Soluble States

All soluble states have a first order elimination route. The rate may be modeled as first order and non-saturable. For large proteins in the plasma, this is typically fluid phase-pinocytosis from endothelial cells and hematopoietic cells (e.g. monocytes). For smaller proteins, this is typically renal filtration. Elimination can happen in any compartment. Typically, elimination in compartments other than the central is assumed either to be zero or equal to that in the central compartment. Soluble proteins that bind to receptors will also be cleared when the receptor ligand complex is internalized and degraded.

Reaction Schema: 0-kelm_<sol>_<cpt>-><Sol>

Relationships: It is convenient to specify elimination rates in terms of equivalent half-lives: kelm_<sol>_<cpt>=ln(2)/Thalf_<sol>_<cpt>

### Internalization and Degradation of Membrane States

Like soluble proteins, the synthesis of membrane proteins are assumed to be made with zeroth-order kinetics and cleared with first order kinetics. The synthesis and elimination rates are computed from the macro-parameters that set the initial steady state for receptor expression (CSS) and for receptor turnover (Thalf). Note that receptors that bind to drug or to ligands to form complexes will also turn over at the same rate as the free receptor. In the case of a multivalent drug, the whole complex will internalize with a rate equal to the sum of the rates of the individual receptors. In some models this will eliminate the entire complex, in other cases one or more of the membrane receptors or drugs will be recovered.

### Computing the Initial Conditions

The initial condition of receptors and ligands is computed by solving for the value of the micro parameters such that the steady state of the model is consistent with the macro parameters provided. For example in a multicompartment model with receptor binding for each state and each compartment there is an equation that can be solved. For example:

$$0 = {ksynth}_{i} - C s {s}_{i} {kel}_{i} - C s {s}_{i} {Rss}_{i} kon + {CRss}_{i} koff - {Css}_{i} {\sum }_{j}^{j \neq i} k t r a n {s}_{i , j} + {\sum }_{j}^{j \neq i} C s {s}_{j} {ktrans}_{j , i}$$

The EFA system of these equations can be solved symbolically to determine the initial steady state. In some instances, this relationship may be simplified using limiting relationships such as the non-depletion binding equation, separation of time scales, or other model reduction methods in order to simplify this equation. This could be to provide closed form solutions or to improve computational performance.

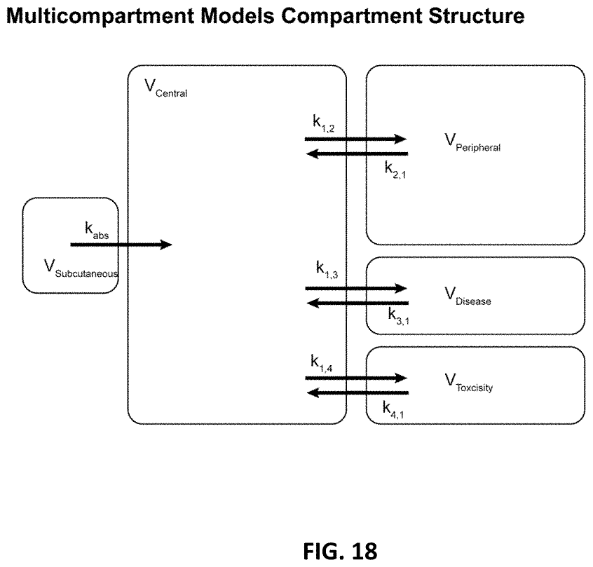

In some implementations, the QSP models in the EFA system include multicompartment models. These models can be divided into 4 reaction compartments. Within each compartment, the reactants are assumed to be well mixed, or more specifically that the activity of the reactants can be related to the amount of the reactants divided by the compartment volume. The two exceptions to this are membrane-membrane binding and membrane-membrane trans binding reactions, which are described in detail in the binding reactions section. In some implementations, the QSP models support arbitrary compartment structures. Models with other connectivity can also be deployed and analyzed. For example, 1 and 2 compartment models, physiologically based pharmacokinetic models (PBPK), or minimal PBPK models. FIG. 18 is a diagram illustrating the connectivity between compartments for the example four compartment QSP models, in some embodiments.

TABLE 46
Example Compartment Volumes Parameters

| Compartment | Definition |
|---|---|
| Central | In a 1 compartment model, the central compartment is the only compartment, |
| | and it typically represents the steady state volume of distribution of the drug. |
| | The default value is 5 L. |
| | In a 2+ compartment model, the central compartment represents the acellular |
| | component of the peripheral blood and can be compared to plasma or serum |
| | PK measurements. For a typical adult male (70 kg, S = 1.9 m²), and adult |
| | female (60 kg, S = 1.6 m²) this is 3.0 and 2.3 L respectively. (Pearson 1995) |
| | The default value in most EFA models is 2.5 L. |
| Peripheral | Represents the non-blood fluids that the antibody can distribute to. This |
| | compartment is less well defined than the central compartment and in reality |
| | represents a collection of individual compartments with different volumes and |
| | exchange rates with the plasma. As such, it is typically approximated as either |
| | (1) the same volume as the central compartment, (2) a function of the |
| | intercompartmental distribution parameters such that at steady state the drug |
| | concentration is the same concentration in central and peripheral |
| | compartments, (3) an approximate physiologic volume computed from the |
| | sum of all of the interstitial fluid volumes for the major organs. |
| | The default value for this compartment is set to 12.8 L. This value is |
| | derived by subtracting the default volumes of the tox and the disease |
| | compartments (0.1 L each) from the value of 13L described in Shah and |
| | Betts. |
| Disease | This represents the interstitial fluids of the diseased tissue. In general, this |
| | would also include the lymph. This may be whole organs or specific involved |
| | regions of an organ. In the case of a whole organ, the default value for the |
| | volume should be the volume of the interstitial fluid of the relevant organ from |
| | Shah and Betts 2012. In the case of involved tissues, this may either be the |
| | proportional fraction of the tissue that is involved: |
| | V_{involved, isf} = V_{organ, isf} * V_{involved, total}/V_{organ, total} |
| | Or by a tissue specific calculation: |
| | V_{involved, isf} = V_{organ, total} * (1 − f_{cellular} − f_{blood} − f_{solid}) |
| | The most common of the second kind is tumors where we typically assume |
| | f_{cellular} = 0.8, f_{blood} = 0, and f_{solid} = 0. |
| | The default value is 0.1 L which corresponds to a 0.5 L solid tissue with |
| | 80% cellularity. |
| Tox | This represents the compartment where the tox pharmacology happens |
| | (assuming it is different from the disease compartment). The volume of this |
| | compartment is computed in the same way as the disease compartment. |
| | The default value is 0.1 L which corresponds to a 0.5 L solid tissue with |
| | 80% cellularity. |

### Steady State Volume of Distribution

In multicompartment models, the steady state volume of distribution can be computed using the individual compartment volumes and the partition coefficients.

$${V}_{s s} = {V}_{c} + {\sum }_{i} {P}_{i} {V}_{i}$$

### Soluble Protein Trafficking

In some implementations, many or all soluble proteins are able to traffic between compartments. This allows soluble proteins that are transported to a tissue in the form of drug protein complexes to unbind and equilibrate between compartments. The rate of distribution is determined by Tdist, and the partition coefficients are determined by the initial steady state concentrations.

### Binding Reactions

### Soluble—Soluble Binding

This reaction type involves the binding of two soluble proteins (or protein complexes). It is described as a bimolecular binding step and unimolecular dissociation step. The complex formed in this reaction is also soluble. The fundamental rate constants are lumped together into an equilibrium dissociation constant (Kd). Typically, these will be the same values in all model compartments but the Kds can be modeled as different in different compartments to describe some more sophisticated mechanisms such as pH dependent binding, or different effective affinities due to avidity. To describe this, a multiplier is provided that relates the Kd_central to Kd_compartment.

Reaction Schema: <Sol1>+<Sol2><-kon, koff-><Sol3>

Relationships: Relate elementary rate constants to equilibrium binding constants

$$Kd = koff / kon$$

$$kon = 1 e - 3 {nM}^{- 1} {s}^{- 1}$$

Note that in multicompartment models the EFA can adjust the Kd of individual compartments by using the multiplier parameter. For example, this could be used to model a different apparent affinity in a disease compartment due to avidity and higher target expression.

### Soluble—Membrane Binding

This reaction type involves the binding of one soluble protein (or protein complex) to one membrane complex. It is the same as the Soluble—Soluble reaction type except that one reactant is membrane protein and the product is membrane. Note that even though one of the states in the bimolecular reaction is not soluble, the same on-rate is typically used. This reflects the fact that the default on-rate is significantly slower than the hard sphere due to other factors dominating the on-rate. As such, the reduced diffusivity of one of the reactants (due to being cell bound) does not typically show up as a different on-rate.

### Bivalent Soluble—Membrane Binding

A combination, particularly for antibodies, is soluble binding followed by membrane binding. Here we are assuming random order symmetric binding for the first step and then symmetric binding for the second step. Not that this pattern continues for higher valences (for example 3 or 4) and can be generalized to multiple targets as well. See Defining Avid Binding Using Standardized Chi Factor for the derivation of the kon_2d.

### Reaction Schema:

<Sol1::>+<Mem><-kon, koff-><Sol1:Mem:>
<Sol1::>+<Mem><-kon, koff-><Sol1::Mem>
<Sol1:Mem:>+<Mem><-kon_2d, koff2-><Sol1:Mem:Mem>
<Sol1::Mem>+<Mem><-kon_2d, koff2-><Sol1:Mem:Mem>

### Effective Valency

Valency refers to the number of binding sites on a molecule. For drugs that are monovalent, that means that they have one binding site for the target molecule. Likewise, bivalent has two binding sites. Here we use the term effective valency to account for more complicated binding stoichiometries. For example, if a bivalent antibody binds to a bivalent ligand in a 2 to 2 complex we would say the effective valency is 1.

Defining Avid Binding Using Standardized Chi Factor

For binding reactions between two soluble species a 3-dimensional binding equation is used. For binding reactions that involve multiple steps after first binding to a receptor on a surface a binding equation that accounts for avidity is needed to provide accurate results. In the models presented here a method that treats this as a 2-dimensional compartment where the activity of the reactants is expressed in nmols/area instead of moles/volume.

$$Rate ( nmol ) = \frac{kon , 2 , 2 d * ( drug : receptor 1 , n mol ) * ( receptor 2 , nmol )}{( total cell surface area )}$$

However, unlike more familiar 3-dimensional binding equations where the on-rate can easily be measured (or for protein-protein interactions tends to fall in a narrow range of values so can often be fixed at a standardized value), 2-dimensional binding reactions require a 2-dimensional on rate that is not typically known. Embodiments described here in include a method that takes the well established 3-dimensional on rate and a standardized chi factor (both of which can be estimated from experimental data) and computes the elementary rate constant which is the 2-dimensional on rate.

Some known methods that have attempted to solve this problem do so by introducing a chi factor that is dependent on the experimental context in which it is estimated. The value is not constant across different experimental contexts and so, in some situations, cannot be used directly to bridge between the different experimental contexts. In particular, chi factors estimated from in vitro assays, in some situations, cannot be used directly for in vivo simulations. The standardized chi factor approach defines a dimensionless chi factor under a standard set of assumptions. This can then be used (with appropriate description of the experimental system) to compute the 2-dimensional on-rate for a given system.

If a typical chi factor has been previously estimated, it can be converted to a standardized chi factor by comparing the cell area and cell density where the chi factor was measured to the standardized conditions.

It is scaled such that standardized chi factor=0 corresponds to single arm binding (i.e. no second binding step), and a chi factor of 1 corresponds to identical and independent binding under typical cell receptor concentrations. Chi factors greater than 1 correspond to increased binding due to favorable 2-dimensional binding.

### Defining the Standardized Conditions

A chi factor is relative to a particular area per cell and cell density (or alternatively total area per fluid volume). The EFA defines the standardized chi factor at cell diameter of 10 um and 1e6 cells/mL. These can correspond to typical mammalian cell sizes and experimental conditions for in vitro studies. The following tables show how the two-dimensional kon rate can be calculated from these quantities. These relationships can also be used to calculate the standardized chi factor from a typical chi factor.

TABLE 47

| id | name | default |
|---|---|---|
| standard_chi | Avidity chi factor | (standard_area_per_cell * |
| | | standard_cell_density)/ |
| | | (area_per_cell * |
| | | default_cell_density) = |
| | | (4*pi*(10/2)\hat{ }2 * 100000/100 * |
| | | 1000*1000)/(4*pi*(10/2)\hat{ }2 * 1e6 * 1000) = 1 |
| | | This makes the models behave the |
| | | same as the fully 3D models when |
| | | the cell density and cell diameter are |
| | | set to the standard values. |
| cell_diameter_um | Cell diameter (um) | 10 |
| cell_density_mL_{compartment} | Cell density (#/mL) | 1e6 |

Constants used in the formula

TABLE 48

| id | value |
|---|---|
| pi | 3.14159265358979323846264338327950288 |
| um2_per_dm2 | 100000*100000 |
| mL_per_L | 1000 |
| uL_per_L | 1000*1000 |
| standard_cell_diameter_um | 10 |
| standard_cells_per_well | 100000 |
| standard_well_volume_uL | 100 |

Algebraic rules used in the model

TABLE 49

| id | definition |
|---|---|
| standard_area_per_cell_um2 | 4 * pi * (standard_cell_diameter_um/2) \hat{ } 2 |
| standard_area_per_cell | standard_area_per_cell_um2/um2_per_dm2 |
| standard_cell_density | standard_cells_per_well/standard_well_volume_uL * uL_per_L |
| kon_2D | kon * standard_chi * standard_area_per_cell * |
| | standard_cell_density |
| area_per_cell_um2 | 4 * pi * (cell_diameter_um/2) \hat{ } 2 |
| area_per_cell | area_per_cell_um2/um2_per_dm2 |
| cell_density_{compartment} | cell_density_mL_{compartment} * mL_per_L |
| total_cells_{compartment} | cell_density_{compartment} * volume_{compartment} |
| area_{compartment} | total_cells_{compartment} * area_per_cell |

### Thermodynamic Constraints

Where there are more than two binding sites in an avid reaction, there are additional constraints on the two dimensional on rate and thus on the standardized chi factor, because there are thermodynamic cycles. This method shows how to identify the thermodynamic cycles and account for them by removing degrees of freedom from the model that would violate a thermodynamic constraint.

Name the receptors A and B. Assume that the drug bound to a particular number of A receptors and B receptors has a certain free energy. Then the EFA can construct a grid of the nine possible free energies:

TABLE 50

| | | |
|---|---|---|
| 0 A, 0 B | 0 A, 1 B | 0 A, 2 B |
| 1 A, 0 B | 1 A, 1 B | 1 A, 2 B |
| 2 A, 0 B | 2 A, 1 B | 2 A, 2 B |

There are 9 configurations, or free energies. Each edge between two cells is a binding reaction with a KD, and the KD is a function of the free energy change between the two cells (RT ln K_{D,ij}=Gⱼ−Gᵢ for configurations i and j).

There are 12 edges corresponding to 12 binding reactions and 12 KDs, but really there are only 9 degrees of freedom (originating from the 9 free energies) if staying consistent with thermodynamic cycles.

Assuming the same koff for all unbinding events for A and the same koff for all unbinding events for B, each KD defines a unique kon for that reaction, and the EFA can set up the constraints in terms of kons instead of KDs.

Assuming that free drug binds its first receptor with kon,A for A and kon,B for B.
0 A, 0 B->1 A, 0 B@konA
0 A, 0 B->0 A, 1 B@konB

If the grid of states is seen as a graph, and the cycles of that graph are the thermodynamic cycles of the system. There are many possible cycles, but we don't need to find them all. Instead, we can identify what is called a cycle basis, which is a set of cycles that can be combined with one another to form all possible cycles. If we satisfy the constraints of the cycle basis, we satisfy all the thermodynamic constraints.

To find a cycle basis, first we construct a spanning tree, which consists of the set of reactions that can reach all states. One such tree is shown in Table 51:

TABLE 51

| | | | | |
|---|---|---|---|---|
| 0 A, 0 B | 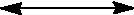 | 0 A, 1 B | 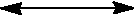 | 0 A, 2 B |
| 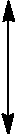 | | 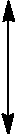 | | 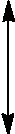 |
| 1 A, 0 B | | 1A, 1 B | | 1 A, 2 B |
| 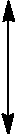 | | 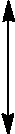 | | 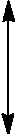 |
| 2A, 0 B | | 2 A, 1 B | | 2 A, 2 B |

Then we take each reaction that's not part of the spanning tree (shown in red in the above diagram) and construct a cycle containing each excluded reaction one at a time. The resulting set of cycles includes a cycle basis. There are four reactions not in the spanning tree. I construct the four following cycles:

TABLE 52

| | | | | | |
|---|---|---|---|---|---|
| 1. | 0 A, 0 B | 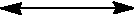 | 0 A, 1B | | 0A, 2 B |
| | 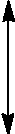 | | 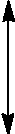 | | |
| | 1 A, 0 B | 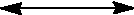 | 1 A, 1 B | | 1 A, 2 B |
| | 2 A, 0 B | | 2 A, 1 B | | 2 A, 2 B |
| 2. | 0 A, 0 B | 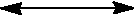 | 0 A, 1 B | | 0 A, 2 B |
| | 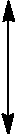 | | 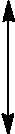 | | |
| | 1 A, 0 B | | 1 A, 1 B | | 1 A, 2 B |
| | 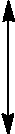 | | 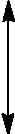 | | |
| | 2 A, 0 B | 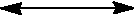 | 2 A, 1 B | | 2 A, 2 B |
| 3. | 0 A, 0 B | | 0 A, 1 B | 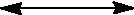 | 0 A, 2 B |
| | | | 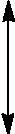 | | 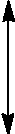 |
| | 1 A, 0 B | | 1 A, 1 B | 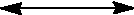 | 1 A, 2 B |
| | 2 A, 0 B | | 2 A, 1 B | | 2 A, 2 B |
| 4. | 0 A, 0 B | | 0 A, 1 B | 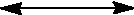 | 0 A, 2 B |
| | | | 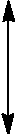 | | 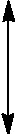 |
| | 1 A, 0 B | | 1 A, 1 B | | 1 A, 2 B |
| | | | 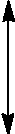 | | 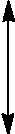 |
| | 2 A, 0 B | | 2 A, 1 B | 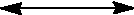 | 2 A, 2 B |

The EFA process includes using these four cycles to evaluate possible binding parameterizations. While there is potential for 9 degrees of freedom, it is uncommon to have data on the energies of all of the microstates. As such, a simplified model with a reduced number of parameters if often more useful in modeling. Presented next are two simplifications that obey the thermodynamic constraints with a significantly reduced number of parameters in particular one and two.

In some implementations, one avidity parameter can be used as shown in Table 53.

TABLE 53

| | | | | |
|---|---|---|---|---|
| 0 A, 0 B | konB | 0 A, 1 B | kon2B | 0 A, 2 B |
| konA | | kon2A | | |
| 1 A, 0 B | kon2B | 1 A, 1 B | kon2B | 1 A, 2 B |
| kon2A | | kon2A | | |
| 2 A, 0 B | kon2B | 2 A, 1 B | kon2B | 2 A, 2 B |

One possible binding parameterization sets one avid binding kon for A and one avid binding kon for B. In this parameterization, if the unbound drug state is already bound to a receptor, we use kon2A for binding to A and kon2B for binding to B. As we'll see, thermodynamic constraints constrain kon2B=kon2A*konB/konA.

The binding system is as follows:
X A, y B->x+1 A, y B@kon2A
X A, y B->x A, y+1 B@kon2B

Here are the equations for the four cycles outlined above:

$$konA * kon 2 B = konB * kon 2 A$$

$$konA * kon 2 A * kon 2 B = konB * kon 2 A * kon 2 A$$

$$kon 2 A * kon 2 B = kon 2 B * kon 2 A ( trivially true )$$

$$kon 2 A * kon 2 A * kon 2 B = kin 2 B * kon 2 A * kon 2 A ( trivially true )$$

In some implementations, only the first two cycles are used. From the first,

$$kon 2 B = kon 2 A * konB / konA$$

The second is equal to the first times kon2A, so the first constraint also satisfies the second.

In some implementations, three avidity parameters can be used as shown in Table 54.

TABLE 54

| | | | | |
|---|---|---|---|---|
| 0 A, 0 B | konB | 0 A, 1 B | kon2BB | 0 A, 2 B |
| konA | | kon2BA | | kon2BA |
| 1 A, 0 B | kon2AB | 1 A, 1 B | kon2BB | 1 A, 2 B |
| kon2AA | | kon2AA | | kon2AA |
| 2 A, 0 B | kon2AB | 2 A, 1 B | kon2BB | 2 A, 2 B |

Another possible binding system initially assigns four avidity parameters:
kon2AA, for binding to A when already bound to A
kon2BB, for binding to B when already bound to B
kon2AB, for binding to B when not yet bound to B but already bound to A
kon2BA, for binding to A when not yet bound to A but already bound to B

The thermodynamic cycles will show that kon2AB=kon2BA*konB/konA, leaving three independent binding avidity parameters.

Here are the cycles' equations:

$$konA * kon 2 AB = konB * kon 2 BA$$

$$konA * kon 2 AA * kon 2 AB = konB * kon 2 BA * kon 2 AA$$

$$kon 2 BA * kon 2 BB = kon 2 BB * kon 2 BA ( trivially true )$$

$$kon 2 BA * kon 2 AA * kon 2 BB = kon 2 BB * kon 2 BA * kon 2 AA ( trivially true )$$

The first cycle implies that kon2AB=kon2BA*konB/konA. The second cycle reduces to the first when kon2AA from both sides is eliminated.

### Cell Engager Model

One model that makes use of the avid binding includes the Cell engager model. This is similar to the multicompartment bispecific model but this model contains two types of avidity cis-avidity (binding between multiple receptors on one cell) and trans-avidity (binding across a cell cell interface). It can use the two parameter version of the three avidity parameters (one cis avidity chi factor for each cell target, and one trans avidity chi factor for the binding between cells).

This is a 4-compartment model with additional disease and tox compartments of an antibody drug candidate that can be given subcutaneously or intravenously. The drug crosslinks a T cell receptor (TCR) on a T cell with a tumor-associated antigen (TAA) on a target cell. It is specifically a solid-tumor model when applicable.

In some cases the model can also be deployed with 1, 2, 3 or more compartments. These can represent solid or liquid tumors. The compartments can be based on PBPK or minimal PBPK compartments.

In some implementations, the Cell Engager Model includes compartments structure as shown in FIG. 18. The model can have four pharmacological compartments: central, peripheral, tumor, and tox. Each pharmacological compartment is implemented by two model compartments: a 3D solution and 2D membrane. The volume of each solution compartment is directly parameterizable. There are two cell types, both of which exist in all pharmacological compartments. The cell density of each cell type is parameterizable. Each cell type has a cell diameter that is parameterizable. The surface area of each cell assumes that it is a sphere. In any 2D reaction where neither reactant is cell-cross-linking but the product is cell-cross-linking. The area of the membrane compartment is equal to the sum of the cell surface areas of both cell types. In other 2D reactions, the area of the membrane compartment is the cell surface area of the free receptor reactant's cell type. In other 2D reactions, the area of the membrane compartment is equal to the sum of the cell surface areas of both cell types.

TABLE 55

| id | name | units | default |
|---|---|---|---|
| volume_central | Volume central | L | 2.5 |
| volume_peripheral | Volume peripheral | L | 12.8 |
| volume_disease | Volume tumor | L | 0.1 |
| volume_tox | Volume tox | L | 0.1 |
| cell_1_diameter | Tumor cell diameter | um | 15 |
| cell_2_diameter | T cell diameter | um | 7 |
| cell_1_density_central | Target cell density | #/mL | 0 |
| | central | | |
| cell_1_density_peripheral | Target cell density | #/mL | 0 |
| | peripheral | | |
| cell_1_density_disease | Tumor cell density | #/mL | 100000000 |
| cell_1_density_tox | Target cell density | #/mL | 0 |
| | tox | | |
| cell_2_density_central | T cell density | #/mL | 1170000 |
| | central | | |
| cell_2_density_peripheral | T cell density | #/mL | 20200000 |
| | peripheral | | |
| cell_2_density_disease | T cell density | #/mL | 1000000 |
| | tumor | | |
| | | | Assumed |
| | | | nominal E:T |
| | | | ratio of 1:100 |
| Cell_2_density_tox | T cell density | #/mL | 1000000 |
| | tox | | Assumed same |
| | | | as disease |

In some implementations, the cell engager model includes multiple (e.g., two) membrane-bound receptor species. Each cell type expresses one and only one of the receptor types. Each receptor is synthesized in a zeroth-order reaction parameterizable according to the receptor density. Each receptor is internalized in a first-order reaction parameterizable according to the receptor half life. There is no transport of either receptor between compartments. There is also an arbitrary scaling factor for the drug:receptor complex elimination rate for each target in each compartment. Each receptor bound to drug still undergoes internalization. The rate is the same as free receptor. Only the drug and the particular receptor are destroyed; all other receptors in the complex are released. Table 56 lists a set of model parameters and a typical value for a T cell engager.

TABLE 56

| id | name | units | default |
|---|---|---|---|
| rec_1_halflife | TAA first order T ½ | hr, min | 1 hr |
| rec_2_halflife | TCR first order T ½ | hr, min | 20 hr |
| rec_1_density_central | TAA density central | #/cell, nM | 10000/cell |
| rec_1_density_peripheral | TAA density peripheral | #/cell, nM | 10000/cell |
| rec_1_density_disease | TAA density tumor | #/cell, nM | 10000/cell |
| rec_1_density_tox | TAA density tox | #/cell, nM | 10000/cell |
| rec_2_density_central | TCR density central | #/cell, nM | 62000/cell |
| rec_2_density_peripheral | TCR density peripheral | #/cell, nM | 62000/cell |
| rec_2_density_disease | TCR density tumor | #/cell, nM | 62000/cell |
| rec_2_density_tox | TCR density tox | #/cell, nM | 62000/cell |
| scale_ab_rec_1_halflife_central | Drug: TAA T ½ scale | unitless | 1 |
| | central | | |
| scale_ab_rec_1_halflife_peripheral | Drug: TAA T ½ scale | unitless | 1 |
| | peripheral | | |
| scale_ab_rec_1_halflife_disease | Drug: TAA T ½ scale tumor | unitless | 1 |
| scale_ab_rec_1_halflife_tox | Drug: TAA T ½ scale tox | unitless | 1 |
| scale_ab_rec_2_halflife_central | Drug: TCR T ½ scale central | unitless | 1 |
| scale_ab_rec_2_halflife_peripheral | Drug: TCR T ½ scale | unitless | 1 |
| | peripheral | | |
| scale_ab_rec_2_halflife_disease | Drug: TCR T ½ scale tumor | unitless | 1 |
| scale_ab_rec_2_halflife_tox | Drug: TCR T ½ scale tox | unitless | 1 |

In some implementations, the cell engager model includes shedding parameters. Each free receptor sheds into a soluble form parameterizable via a steady-state concentration. Receptor bound in a complex does not shed. The shed receptors eliminate from the central compartment according to a first-order reaction. Shed receptors do not eliminate from other compartments. The shed receptors transport between the central compartment and the other compartments according to first-order reactions.

TABLE 57

| id | name | units | default |
|---|---|---|---|
| shed_1_density_{compartment} | Shed TAA density | nM | 0 |
| | {compartment} | | |
| shed_2_density_{compartment} | Shed TCR density | nM | 0 |
| | {compartment} | | |
| shed_1_halflife | Shed TAA T ½ | min | 30 |
| shed_2_halflife | Shed TCE T ½ | min | 30 |
| shed_1_tdist_{compartment} | Shed TAA Tdist | hr | 30 |
| | {compartment} | | |
| shed_2_tdist_{compartment} | Shed TCR Tdist | hr | 30 |
| | {compartment} | | |

In some implementations, the cell engager model includes parameters associated with dosing and routes of administration. The model supports at least the following:

Two routes of dosing: SC and IV;

IV is a bolus dose applied directly to the central compartment;

SC is a bolus dose applied to a separate subcutaneous compartment;

Drug in the subcutaneous compartment is absorbed into the central compartment according to a first-order reaction;

The dose amount is parameterized in units of mg or mg/kg of body weight;

Body weight will itself is parameterizable;

The dose interval is parameterizable with a dropdown menu of standard pharmacological intervals; and

The numbers of doses are parameterizable.

TABLE 58

| id | name | units | default |
|---|---|---|---|
| interval | Interval | dropdown | Q2W |
| dose | Dose | mg, mg/kg | 1 mg |
| dose_count | Number of doses | integer | 7 |
| mw | Molecular weight | Da | 150000 |
| bw | Body weight | kg | 70 |
| absorption_half_life | SC absorption T ½ | d | 2.5 |

In some implementations, the cell engager model includes elimination parameters. Free drug is eliminated from any compartment in a first-order reaction parameterizable with a half life. There is also an arbitrary scaling factor for the elimination half life in each compartment. Drug:shed complexes are eliminated at the same rate as free drug

TABLE 59

| id | name | units | default |
|---|---|---|---|
| ab_halflife | Biological | d | 14 |
| | first-order T ½ | | |
| scale_ab_halflife_central | Soluble Drug T ½ | unitless | 1 |
| | scale central | | |
| scale_ab_halflife_peripheral | Soluble Drug T ½ | unitless | 1 |
| | scale peripheral | | |
| scale_ab_halflife_disease | Soluble Drug T ½ | unitless | 1 |
| | scale disease | | |
| scale_ab_halflife_tox | Soluble Drug T ½ | unitless | 1 |
| | scale tox | | |

In some implementations, the cell engager model includes valency parameters. The drug is bispecific, with separate binding arms for each receptor. Each binding arm may be monovalent or bivalent, parameterized with a dropdown menu.

TABLE 60

| id | name | units | default |
|---|---|---|---|
| ab_valency_1 | Effective valency to TAA | dropdown | 1 |

In some implementations, the cell engager model includes transport parameters. Free drug is transported between the central compartment and each of the other compartments. There is no transport directly between the other compartments. Each transport reaction is parameterized with a Tdist in units of hr and a unitless Pdist. Drug:shed complexes are transported at the same rate as the free drug. Drug bound to membrane receptor is not transported

TABLE 61

| id | name | units | default |
|---|---|---|---|
| ab_tdist_peripheral | Drug Tdist peripheral | hr | 30 |
| ab_tdist_disease | Drug Tdist tumor | hr | 30 |
| ab_tdist_tox | Drug Tdist tox | hr | 30 |
| ab_pdist_peripheral | Drug Pdist peripheral | unitless | 0.190625 |

In some implementations, the cell engager model includes 3D Binding parameters. When free drug binds to either receptor, it is a 3D second-order reaction. The 3D kon is constant with a value 1e-3/nM/s. The koff of each binding reaction is parameterized via a Kd with units of nM.

TABLE 62

| id | name | units | default |
|---|---|---|---|
| ab_kd_1 | Drug: TAA KD | nM | 1 |
| ab_kd_2 | Drug: TCR KD | nM | 100 |
| scale_ab_kd_1_central | Drug: target KD scale | unitless | 1 |
| | central | | |
| scale_ab_kd_1_peripheral | Drug: target KD scale | unitless | 1 |
| | peripheral | | |
| scale_ab_kd_1_disease | Drug: target KD scale | unitless | 1 |
| | disease | | |
| scale_ab_kd_1_tox | Drug: target KD scale | unitless | 1 |
| | tox | | |
| scale_ab_kd_2_central | Drug: CD3 KD scale | unitless | 1 |
| | central | | |
| scale_ab_kd_2_peripheral | Drug: CD3 KD scale | unitless | 1 |
| | peripheral | | |
| scale_ab_kd_2_disease | Drug: CD3 KD scale | unitless | 1 |
| | disease | | |
| scale_ab_kd_2_tox | Drug: CD3 KD scale | unitless | 1 |
| | tox | | |

In some implementations, the cell engager model includes 2D binding parameters. All kons of 2D binding reactions are parameterized via avidity according to the standardized chi factor method described above. The model can be configured with a 1 to 1, 1 to 2, 2 to 1, and 2 to 2, where the first number is the number of binding sites on the effector cell (for example CD3 on a T cell) and the second number is the number of tumor associated antigen sites (TAA). Note that these sites may be on tumor cells (or other target cells in the case of indications other than cancer) as well as non-tumor cells, which may represent on-target off tumor toxicity. In the case of a 1 to 2 configuration of the model, which is typical of T cell Engagers, then Table 63 lists a parameterization with default values.

TABLE 63

| id | name | units | default |
|---|---|---|---|
| standard_cis_chi | Cis avidity chi factor | unitless | 30000 |
| standard_trans_chi | Trans avidity chi factor | unitless | 30000 |

The EFA process can be executed by a processor based on instructions/code stored in a memory (e.g., processor 111 or memory 112 at the server 101 as discussed with respect to FIG. 1). Early Feasibility Assessment (EFA) can be implemented as an interactive UI or API-driven application that helps users assess the therapeutic characteristics (e.g., half life, affinity) used to achieve success criteria given the predetermined target profile (e.g., dose, administration, frequency). Early R&D poses many questions when determining if a therapeutic enters the portfolio, and if it does, how difficult (from a discovery and engineering perspective) it will be to develop.

In some implementations, the EFA system can include an intuitive, interactive application that allows researchers to identify what drug parameters are required, identify what biological parameters are important, de-risk a project by highlight challenges early-on, determine what experiments are most important, and/or more advantages. In some implementations, the EFA system can be used to provide rational Go/No-go guidance, optimize and/or improve screening funnels for Lead Generation, prioritize key experiments/data, assess risk earlier, align stakeholders earlier, and manage resources.

The EFA system can provide a library of pre-built monospecific and bispecific, single compartment and multiple compartment models covering most common biotherapeutic pharmacology. The EFA system can provide models describing advanced pharmacology just as T-cell engager models, and biotherapeutic models with avidity, as well as user custom models that can be deployed to support more specialized pharmacology. The EFA system can assess feasibility of drugs that are agonist, antagonist, have variable drug valency, differ in their binding, affinity, avidity, molecular weight, have variable elimination and biodistribution rates, different routes of administration (for example subcutaneous injection, intravenous infusion, bolus injection, continuous infusion, intramuscular injection, oral). Models are right sized and include many mechanisms that can prevent the feasibility for a drug target. This includes the presence of shed receptor, variable ligand concentration and turnover, receptor density and turnover, ligand-receptor affinity, and/or the like.

The EFA system can enable easy exploration of “What If” questions with scenarios. The EFA system can, via the user interface, create Scenarios, or sets of parameters, to easily contrast and compare various scenarios. The EFA system can add, duplicate, or delete scenarios with the click of a button in the EFA er interface. The EFA system can allow the users to vary parameters in the scenario table to explore risk and uncertainty while providing high-performance computing to support near real-time simulation when any scenario is selected.

Gain Deeper Insight with Built-in Analyses and Interactive Plots. The EFA system can perform 1- or 2-dimensional analyses by selecting scan parameters and output feasibility criteria including percent Inhibition, Activation, and Target Engagement. Visually assess results and key parameter values by interacting with dose-response and pharmacokinetic and pharmacodynamic (PK/PD) plots.

Document, Share, and Reproduce Analyses. The EFA system can save Scenario sets for traceability and reproducibility and/or to share work with colleagues. The EFA system can automatically generate a report with a unique ID and date and time stamp and download plots individually as PNG images.

Models and analysis methods are optimized and/or configured to be fast and numerically stable. The models and analysis in the EFA system can be designed to be right sized for answering scientific questions in the early drug discovery process. Models and methods are implemented as to have sufficient mechanism, while still being numerically stable and able to be solved accurately and quickly to enable rapid analysis.

Models in the EFA system can be validated by comparing model predictions to real world data from approved therapeutics. The server (or the computing devices) can verify and/or validate the models in the EFA system. Verification ensures that the models perform as expected numerically, and overall model input output behavior is as expected (for example, such as equilibrium conditions, mass conservation relationships, and timescales). Validation ensures that models match real world data for therapeutics. Where possible data on approved therapeutics are used. Where that is not possible, the most relevant available data can be used (such as clinical data from clinical trials, or preclinical data from relevant species). Table 64 shows example model validation data for drugs that bind to soluble ligands (Her2 and CD80/CD86, however, are not soluble ligands.)

TABLE 64

| | | | | | | | | Model |
|---|---|---|---|---|---|---|---|---|
| | | | | | | | | Predicted |
| | Drugs | | | | Clinical Dose | | | Dose |
| | | | Kd | T(½) | | | Dose | ID90 |
| Disease | Target | Drug | (pM) | (days) | Schedule | | (mg) | (mg) |
| RA | TNFa | Remicade | 4.2 | 14 | 8 | Weeks | 210 | 184 |
| RA | TNFa | Humira | 8.6 | 14 | 2 | Weeks | 40 | 17 |
| Psoriasis | IL-23/IL-12 | Stelara | 1 | 21.6 | 90 | Days | 45 | 27 |
| Psoriasis | IL-23 | Skyrizi | 1 | 27 | 12 | Weeks | 150 | 172 (119-225) |
| SLE | BLyS (BAFF) | Benlysta | 274 | 19.4 | 1 Week s.c. | | 200 | 130 |
| SLE | BLyS (BAFF) | Benlysta | 274 | 19.4 | 4 Weeks i.v. | | 700 | 878 |
| Asthma | IgE | Xolair | 20 | 26 | 2 | Weeks | 225 | 327 |
| Transplant | CD80/CD86 | Nulojix | 20 | 8.5 | 4 | Weeks | 350-700 | 794 |
| Rejection | | | | | | | | |

Table 65 shows example model validation data for drugs that bind to receptor targets. In this case for different targets different criteria are used based on the mechanism of action of the drug, or the data available for the drug.

TABLE 65

| | | | | | | | | | Model | | |
|---|---|---|---|---|---|---|---|---|---|---|---|
| | | | | | | | | | Simulated | | |
| | Drugs | | | | Clinical Study Dose | | | | Dose | | |
| Disease/ | | | Kd | T(½) | | | Dose | | Dose | | Benchmark |
| Population | Target | Drug | (pM) | (days) | Schedule | | (mg/kg) | | (mg/kg) | | Criteria |
| Healthy | CD28 | FR104 | 600 | 6.8 | Single dose | | 0.005-0.5 | | >0.07 | | 100% peak |
| Volunteers | | | | | | | | | | | receptor |
| | | | | | | | | | | | occupancy |
| Oncology | Her2 | Herceptin | 5000 | 16 | 3 | Weeks | 420 | | 359 | | 90% Target |
| | | | | | | | | | | | Engagement |
| | | | | | | | | | | | (TE90) |
| Oncology | PD-1 | Keytruda | 29 | 22 | 3 | Weeks | 140 | | 2.1* | | ID90. Note PD- |
| Oncology | PD-1 | Opdivo | 2.6 | 25 | 2 | Weeks | 210 | | 11.3* | | 1s did not show |
| | | | | | | | | | | | dose-dependent |
| | | | | | | | | | | | activity in the |
| | | | | | | | | | | | clinic, so dosing |
| | | | | | | | | | | | is likely well |
| | | | | | | | | | | | above ID90 |
| Oncology | CD74 | Milatuzumab | 1000 | 21 | 1 | Day | 1.5-8 | | 4 | | No antibody |
| | | | | | | | | | | | accumulation |
| | | | | | | | | | | | through 8 mpk |
| Oncology | CD33 | Lintuzumab | 200 | 14 | 3 | Day | 0.5-10 | mg/m2 | 3, 10 | mg/m2 | 80-100% peak |
| | | | | | | | | | | | receptor |
| | | | | | | | | | | | occupancy |
| GvHD | PSGL-1 | Neihulizumab | 30,000 | 16 | Single dose | | 3, 6 | mg/kg | 3, 6 | mg/kg | Duration above |
| | | | | | | | | | | | 90% RO |
| Oncology | CD37 | BI 83628 | 1000 | 21 | 2 | weeks | 3-800 | mg | 170 | mg | Dose with |
| | | | | | | | | | | | sustained |
| | | | | | | | | | | | peripheral B- |
| | | | | | | | | | | | cell reduction; |
| | | | | | | | | | | | Nonlinear PK |

Table 66 shows data describing a subset of the receptor targets taken from the literature showing the types of sources used to build an EFA analysis.

TABLE 66

| | | | | | Target Parameters | |
|---|---|---|---|---|---|---|
| | Drug Parameters | | | | Receptor | |
| | | | Half- | Drug | Ligand | Target |
| Drug | Dosing | | life | Affinity | Affinity | Receptor |
| (target) | Schedule | Dose | (T½) | (Kd) | (Kd) | Expression |
| Herceptin | IV, | 420 | 16 | 5 | N/A | 1.9 |
| | Q3W | mg | days | nM | | nM |
| (Her2) | HERCEPTIN | | | HERCEPTIN | | Estimated from |
| | (trastuzumab) | | | (trastuzumab) | | dose needed to |
| | Label - FDA | | | Label - FDA | | saturate |
| | | | | | | TMDD |
| Nulojix | IV, | 5-10 | 8.5 | 20 | 2.5 | 0.22 |
| | Q1M | mg/kg | days | pM | nM | nM |
| (CD80/CD86) | Shen et al. Clinical Drug | | | | Linsley | Target |
| | Investigation 2014; Larsen et | | | | et al. | Burden |
| | al. Transplantation 2010; | | | | Immunity | Calculation |
| | package insert | | | | 1994 | |
| Keytruda | IV, | 140 | 22 | 29 | 560 | 8.3 |
| | Q3W | mg | days | pM | nM | pM |
| (PD-1) | BLA | | | | Butte | Target |
| | | | | | et al. | Burden |
| | | | | | Immunity | Calculation |
| | | | | | 2007 | |
| Opdivo | IV, | 210 | 25 | 3 | 560 | 8.3 |
| | Q2W | mg | days | nM | nM | pM |
| (PD-1) | BLA | | | | Butte | Target |
| | | | | | et al. | Burden |
| | | | | | Immunity | Calculation |
| | | | | | 2007 | |
| FR104 | IV, | 0.005-0.5 | 6.8 | 0.6 | 4000 | 0.32 |
| | single | mg/kg | days | nM | nM | nM |
| | dose | | | | | |
| (CD28) | Poirier et al. American | | | | vander | Target |
| | Journal of Transplantation | | | | Merwe | Burden |
| | 2012; Poirier et al. J | | | | et al. | Calculation |
| | Immunology, 2016 | | | | Journal of | |
| | | | | | Experimental | |
| | | | | | Medicine | |
| | | | | | 1997 | |
| Milatuzumab | IV, | 1.5, 3, | 16 | 0.3-3 | 9 | 2.1 |
| | Q1D | 6, 8 | days | nM | nM | nM |
| | | mg/kg | | | | |
| (CD74) | Martin et al. | | Typical | Assume | Leng | Target |
| | Leukemia & | | values for | 0.3-3 nM | et al. | Burden |
| | Lymphoma | | antibodies | (based on in | J Exp | Calculation |
| | 2015 | | | vitro study | Med | |
| | | | | conc. of | 2003 | |
| | | | | 30 nM) | | |
| Lintuzumab | IV, | 0.5-10 | 16 | 0.2 | N/A | 1.8 |
| | Q3D | mg/m2 | days | nM | | nM |
| (CD33) | Caron et al. | | Typical | Co et al. | | Target |
| | Blood 1994 | | values for | Journal of | | Burden |
| | | | antibodies | Immunology, | | Calculation |
| | | | | 1992 | | |
| Neihulizumab | IV, | 3, 6 | 16 | 30 | N/A | 5.6 |
| | single | mg/kg | days | nM | | nM |
| | dose | | | | | |
| (PSGL1) | Neihulizumab | | Typical | 30 nM | | Target |
| | EHA | | values for | reported | | Burden |
| | 2020 poster | | antibodies | KD in | | Calculation |
| | | | | AbGn | | |
| | | | | patent | | |
| BI 83626 | IV, | 3-800 | 21 | 1 nM | N/A | 100 |
| | Q2W | mg | days | | | nM |

| | | | |
|---|---|---|---|
| (CD37) | Stilgenbauer | Typical | Stilgenbauer |
| | et al. | values for | et al. |
| | Leukemia | antibodies | Leukemia |
| | 2019 | (high | 2019; |
| | | end of | Hallek |
| | | typical) | et al. |
| | | | 2018 |
| | | | Blood |

| | Target Parameters | | | | |
|---|---|---|---|---|---|
| | Target | | Ligand | Soluble | Receptor |
| Drug | Receptor | Ligand | Half- | Receptor | Half- |
| (target) | Turnover | Expression | life | Expression | Life |
| Herceptin | 1.2 | N/A | N/A | N/A | N/A |
| | hours | | | | |
| (Her2) | Estimated from | | | | |
| | dose needed to | | | | |
| | saturate | | | | |
| | TMDD | | | | |
| Nulojix | 30 | 0.38 | 120 | N/A | N/A |
| | min | nM | min | | |
| (CD80/CD86) | typical | Target | Egen et al. | | |
| | value | Burden | Nature | | |
| | | Calculation | Immunology | | |
| | | | 2002 | | |
| Keytruda | 120 | 126 | 120 | N/A | N/A |
| | min | pM | min | | |
| (PD-1) | Typical | Target | Herbst | | |
| | value | Burden | et al. | | |
| | | Calculation | Nature | | |
| | | | 2014 | | |
| Opdivo | 120 | 126 | 120 | N/A | N/A |
| | min | pM | min | | |
| (PD-1) | Typical | Target | Herbst | | |
| | value | Burden | et al. | | |
| | | Calculation | Nature | | |
| | | | 2014 | | |
| FR104 | 24 | 0.11 | 30 | 7.2 | 60 |
| | hour | nM | min | nM | min |
| (CD28) | Egen | Target | typical | Hebbar | Typical |
| | et al. | Burden | value | et al. | value |
| | Immunity | Calculation | | Clinical | |
| | 2002 | | | and | |
| | | | | Experimental | |
| | | | | Immunology, | |
| | | | | 2004 | |
| Milatuzumab | 5 | 0.056- | 60 | 0.78 | 60 |
| | min | 2.1 | min | nM | min |
| | | nM | | | |
| (CD74) | Ong et | Krock | Assumption | Soppert | Assumption |
| | al. | enberger | | et al. | |
| | Immunology | et al. | | J Am | |
| | 1999 | Anticancer | | Heart | |
| | | Research | | Assoc | |
| | | 2012 | | 2018 | |
| Lintuzumab | 120 | N/A | N/A | N/A | N/A |
| | min | | | | |
| (CD33) | Typical | | | | |
| | Value | | | | |
| Neihulizumab | 120 | N/A | N/A | N/A | N/A |
| | min | | | | |
| (PSGL1) | Typical | | | | |
| | value for | | | | |
| | receptors | | | | |
| BI 83626 | 5400 | N/A | N/A | N/A | N/A |
| | min | | | | |
| (CD37) | Press | | | | |
| | et al. | | | | |
| | Cancer | | | | |
| | Res | | | | |
| | 1989 | | | | |

Intuitive interface allows non-modelers to directly use the software to answer questions. The QSP models provided by the EFA system can be generated using general purpose scientific programming languages such as MATLAB or R, or using software designed for expert modelers. The EFA system can combine prebuilt models, intuitive interfaces, transparent high performance computing, which allows scientists who are not experts in mechanistic modeling to directly perform analysis. This includes, Project Leaders, Protein Engineers, Protein Chemists, and Biologists.

An EFA system enables users to explore a high dimensional parameter space quickly by building partial factorial parameter space explorations. The models in the EFA system may have hundreds or more parameters. It is not easy to visualize such high dimensional spaces. The EFA system allows the user to quickly develop scenarios which represent low dimensional slices through the higher dimensional parameter space. These can be thought of as partial factorial parameter space studies, similar to the concept of partial factorial experiment design. By making this fast and easy, the user can quickly explore these dimensions and identify which parameters will be impactful to their question.

Low latency computing enables interactive feeling experience. The EFA system leverages parallel computing (or parallel processing) to speed up the calculations for parameter scans. The speedup can be measured as an increase in the total number of simulations that can be performed in a certain time by the system (throughput). However, the speedup can also be measured in the minimum time from when a user requests an action until the results are displayed back to the user (latency). The EFA system can optimize and/or improve the throughput as well as latency to enable users to feel the system is interactive, and to help users develop a more intuitive feeling for what properties of the system different parameters control.

The EFA system can be used to solve the following questions, for example:
Do we target the ligand or the receptor?
Which step of the signaling cascade do we target?
What affinity do we need (e.g. 1 pM vs. 1 nM)?
Selectivity (e.g. 10×, 100×, 1000×) for desired target
How do I optimize my experimental design in the context of study limitations?
What are optimal drug properties?
Which of our preclinical candidates should we move forward?
How does soluble target impact the risk and design?
Is it possible to cover your target inhibition given the preferred dosing regimen? For example can the drug achieve sustained 90% inhibition for >90% simulated patients?
How do you set up a rational screening funnel?
What are they Affinity/Avidity cutoffs (for monos and bispecifics, and their parental mAbs)
What are the PK requirements for my drug?
What model parameters or gaps in the literature most impacts the project's success?
What's most important to PK/TO? Ligand concentration? Receptor density? Internalization? Ligand-Receptor affinity? Across multiple indications if possible?
How do you assess risk and prioritize your portfolio?
How to identify the easiest wins from a developability and knowledge gap point of view?

De-Risking Drug Discovery: Early Feasibility Assessment for Saving Time and Money

Developing new therapeutics can be a costly and time-intensive endeavor, often taking upwards of 10 years and investment of as much as 2 billion USD to take a new agent to market. Much of this cost is related to the attrition of other equally promising projects, and particularly troubling are the late-stage clinical trial failures—more the rule than the exception. Clearly, being able to identify the failures early while picking the “winners” more often is a strong move toward decreasing development costs. This is where early feasibility assessment (EFA) comes in with the ability to drastically improve the efficiency of pharmaceutical spending and accelerate the discovery of potent, appropriately targeted (fewer side effects) molecules and biologics.

Early feasibility assessment can include the application of literature and experimental data to relevant, robust models to predict whether a pharmaceutical intervention has a reasonable chance for success.

A successful therapeutic survives whichever route of administration is chosen, crosses various biological barriers, interacts with one or more targets (perhaps displacing natural ligands), and maintains this interaction for an appropriate duration. Factors such as solubility, membrane permeability, metabolic stability, Kd (kon and koff) of interaction with target, half-life, target molecule(s) concentrations (in the site-of-action compartment), and target inhibition necessary to produce effects will all have significant impacts on the likelihood that a therapeutic can work as intended. Specificity toward the target of interest can also greatly impact the likelihood of clearing regulatory hurdles by limiting side effects.

In some cases, for example, a hundreds-of-nanomolar inhibitor with a low-abundance target can be effective in monthly dosing, whereas a different target, overexpressed in a specific compartment, may only be feasibly targeted by a sub-nanomolar inhibitor with long half-life and daily administration. The difficulty of developing these two hypothetical agents is quite distinct. Knowing at the outset that nothing short of a sub-nanomolar inhibitor with incredible stability could be expected to stand a reasonable chance of success in a given condition, a group can make the appropriate cost-benefit analyses and may choose a different target to pursue. Answering these kinds of questions, early in the process, is at the core of EFA's value proposition.

Lipinski's rule of 5 (ROF) ultimately addresses these same variables in a one-size-fits-all generic approach for small molecules, estimating orally bioavailable drug-like behavior based on a few molecular parameters. Medicinal chemists and development teams are well versed in recognizing concerning values related to absorption, distribution, metabolism, and excretion (ADME). Biologics and natural products stretch beyond the purview of ROF, but their efficacy relies on the same PK/PD principles.

Despite a general understanding of the importance of these parameters, the fact is that few companies have the expertise to create and work with optimized, rigorous mathematical models that more accurately predict how a molecule or biologic will fare in a given disease context, to mine the literature and integrate information from different sources, and to discern the right questions to ask early.

How EFA Modeling Helps

Creating valid quantitative systems pharmacology (QSP) models uses a comprehensive knowledge of the dynamics of molecular interactions in a complex, real biological environment and how these interactions influence disease processes.

In an example use case, a primary target was known, and a set of 5 different secondary targets was being considered. To identify the optimal and/or desired second target, models were prepared, systematically asking whether interacting with both targets in the compartment of interest could be accomplished—modulating the affinity of hypothetical antibodies, the dosing regimen, and antibody half-life and creating time vs. concentration curves for each set of conditions. By performing these calculations, before any antibodies were designed or any experiments were run, key information was gleaned to guide the team's approach. For instance, analysis showed that 3 of the 5 targets could not be feasibly targeted in a once-monthly subcutaneous administration. Additionally, increasing antibody half-life from 21 to 35 days was predicted to greatly impact concentration, mostly independent of dosing regimen, whereas further increasing antibody half-life was likely to have severely diminishing benefits which would likely add expense and would delay the drug development for little or no benefit. This helped the team make informed decisions about the optimal target to pursue and the level of effort to apply to optimizing antibody half-life.

Predictive simulations can answer several crucial questions to guide drug discovery and development. These answers can give a production team a picture of the target product profile (TPP) necessary for efficacy—the ability to say “this is the kind of molecule we need”. Predictive modeling can also help prioritize which experiments to run when literature values are insufficient. In some cases you need to know how many target sites there are, the affinity of natural ligands, and more, and sometimes these are less impactful to potential efficacy. All of this adds up to early de-risking; which experiments are necessary to make the crucial Go/No-Go decisions and avoid driving into dead ends?

In many cases, simulations can be run using similar targets with known drugs/biologics for which real-world data exist. The models can then be benchmarked against real-world data.

While various embodiments have been described and illustrated herein, one will readily envision a variety of other means and/or structures for performing the function and/or obtaining the results and/or one or more of the advantages described herein, and each of such variations and/or modifications is deemed to be within the scope of the embodiments described herein. More generally, one will readily appreciate that all parameters, dimensions, materials, and configurations described herein are meant to be examples and that the actual parameters, dimensions, materials, and/or configurations will depend upon the specific application or applications for which the teachings is/are used. One will recognize, or be able to ascertain using no more than routine experimentation, many equivalents to the specific embodiments described herein. It is, therefore, to be understood that the foregoing embodiments are presented by way of example only and that, within the scope of the disclosure, including the appended claims and equivalents thereto, disclosed embodiments may be practiced otherwise than as specifically described and claimed. Embodiments of the present disclosure are directed to each individual feature, system, tool, element, component, and/or method described herein. In addition, any combination of two or more such features, systems, articles, elements, components, and/or methods, if such features, systems, articles, elements, components, and/or methods are not mutually inconsistent, is included within the scope of the present disclosure.

The above-described embodiments can be implemented in any of numerous ways. For example, embodiments may be implemented using hardware, software or a combination thereof. When implemented in software, the software code can be stored (e.g., on non-transitory memory) and executed on any suitable processor or collection of processors, whether provided in a single computer or distributed among multiple computers.

Further, it should be appreciated that a compute device including a computer can be embodied in any of a number of forms, such as a rack-mounted computer, a desktop computer, a laptop computer, netbook computer, or a tablet computer. Additionally, a computer can be embedded in a device not generally regarded as a computer but with suitable processing capabilities, including a smart phone, smart device, or any other suitable portable or fixed electronic device.

Also, a computer can have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer can receive input information through speech recognition or in other audible format.

Such computers can be interconnected by one or more networks in any suitable form, including a local area network or a wide area network, such as an enterprise network, and intelligent network (IN) or the Internet. Such networks can be based on any suitable technology and can operate according to any suitable protocol and can include wireless networks, wired networks or fiber optic networks.

The various methods or processes outlined herein can be coded as software that is executable on one or more processors that employ any one of a variety of operating systems or platforms. Additionally, such software can be written using any of a number of suitable programming languages and/or programming or scripting tools, and also can be compiled as executable machine language code or intermediate code that is executed on a framework or virtual machine.

In this respect, various disclosed concepts can be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure discussed above. The computer readable medium or media can be transportable, such that the program or programs stored thereon can be loaded onto one or more different computers or other processors to implement various aspects of the present disclosure as discussed above.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but can be distributed in a modular fashion amongst a number of different computers or processors to implement various aspects of the disclosure.

Computer-executable instructions can be in many forms, such as program modules, executed by one or more computers or other devices. Generally, program modules include routines, programs, objects, components, data structures, etc. that perform particular tasks or implement particular abstract data types. Typically the functionality of the program modules can be combined or distributed as desired in various embodiments.

Also, various concepts can be embodied as one or more methods, of which an example has been provided. The acts performed as part of the method may be ordered in any suitable way. Accordingly, embodiments can be constructed in which acts are performed in an order different than illustrated, which can include performing some acts simultaneously, even though shown as sequential acts in illustrative embodiments. All publications, patent applications, patents, and other references mentioned herein are incorporated by reference in their entirety.

What is claimed is:

1. A method, comprising:
receiving, at a processor, a first user input that selects a quantitative system pharmacology (QSP) model from a plurality of QSP models, the QSP model having a plurality of parameters and a set of reaction compartments to characterize biological interactions between a therapeutic drug candidate and a target, the plurality of parameters having a parameter associated with a dosage of the therapeutic drug and being selectable by a second user input;
receiving, at the processor, a third user input associated with (1) values of a first subset of the plurality of parameters and (2) a set of target criteria for a therapeutic purpose;
generating, at the processor and via parallel processing, a set of simulation results based on the (1) QSP model, (2) the values of the first subset of the plurality of parameters, and (3) the set of target criteria,
the generating the set of simulation results includes performing, at the processor, a one-dimensional or a two-dimensional scan of at least one parameter from a second subset of the plurality of parameters from a first value of a set of values to a second value from the set of values;
determining, at the processor, if at least one value of the at least one parameter exists that satisfies the set of target criteria for the therapeutic drug candidate and the target for the therapeutic purpose; and
sending, from the processor, the set of simulation results and the at least one value of the at least one parameter when the at least one value exists.

2. The method of claim 1, further comprising:
presenting the set of simulation results in an interactive graphical user interface that allows a user to adjust the plurality of parameters and the set of target criteria to analyze a correlation between the plurality of parameters and the set of target criteria.

3. The method of claim 1, wherein the first subset of the plurality of parameters includes a nominal macro parameter from a set of nominal macro parameters, the set of nominal macro parameters including at least one of a time constant, a concentration of one or more states of the QSP model, or a thermodynamic constant.

4. The method of claim 1, further comprising:
retrieving values of a third subset of the plurality of parameters from a data storage;
the generating the set of simulation results includes generating the set of simulation results based on the values of the third subset of the plurality of parameters.

5. The method of claim 1, wherein:
the generating the set of simulation results includes generating a set of partial factorial expansion of a high dimensional parameter space.

6. The method of claim 1, wherein:
the generating the set of simulation results includes generating a set of partial factorial expansion of a high dimensional parameter space;
the generating the set of simulation results includes adjusting the high dimensional parameter space by decimating un-needed dimensions.

7. The method of claim 1, wherein:
the generating the set of simulation results is in response to the first user input and the third user input.

8. The method of claim 1, wherein:
the determining if at least one threshold value of the at least one parameter exists includes using a binary search, a bisection search, Newton's method, or a variable order interpolation.

9. The method of claim 1, wherein:
the set of simulation results is cached based on a hash of a parameter vector to enable acceleration of repeated simulations.

10. The method of claim 1, wherein:
the generating the set of simulation results is performed in a distributed computing environment.

11. The method of claim 1, wherein:
the generating the set of simulation results is performed in a distributed computing environment,
the communication in the distributed computing environment is mediated by a task queue.

12. The method of claim 1, wherein the QSP model includes characterization of the biological interactions associated with a T cell receptor on T cells crosslinked to a tumor-associated antigen on tumor cells and normal cells.

13. The method of claim 1, wherein the therapeutic purpose is to determine feasibility of developing the therapeutic drug.

14. The method of claim 1, further comprising:
comparing the at least one value of the at least one parameter with a set of predetermined values of the at least one parameter to determine feasibility of developing the therapeutic drug.

15. A non-transitory processor-readable medium storing code representing instructions to be executed by a processor, the code comprising code to cause the processor to:
receive a user input that selects a quantitative system pharmacology (QSP) model from a plurality of QSP models, the QSP model having a plurality of parameters, the QSP model having a set of reaction compartments to characterize biological interactions between a therapeutic drug candidate and a target in biological environments;
generate a set of simulation results based on (1) the QSP model, (2) values of a set of macro parameters from the plurality of parameters, and (3) a set of target criteria,
the code to cause the processor to generate the set of simulation results includes code to scan, from a first value of a set of values to a second value from the set of values, at least one parameter from a subset of the plurality of parameters;
determine if at least one value of the at least one parameter exists that satisfies the set of target criteria for the therapeutic drug candidate and the target for a therapeutic purpose; and
send the set of simulation results and the at least one value of the at least one parameter when the at least one value exists.

16. The non-transitory processor-readable medium of claim 15, wherein:
the set of reaction compartments includes a single compartment associated with a steady state volume of distribution of the therapeutic drug candidate.

17. The non-transitory processor-readable medium of claim 15, wherein:
the set of reaction compartments includes a first reaction compartment and a second reaction compartment;
the first reaction compartment is associated with acellular components of peripheral blood; and
the second reaction compartment is associated with non-blood fluids to which the therapeutic drug candidate binds.

18. The non-transitory processor-readable medium of claim 15, wherein:
the set of reaction compartments includes a first reaction compartment and a second reaction compartment;
the first reaction compartment is associated with acellular components of peripheral blood; and
the second reaction compartment is associated with interstitial fluids of diseased tissue.

19. The non-transitory processor-readable medium of claim 15, wherein:
the set of reaction compartments includes a first reaction compartment and a second reaction compartment;
the first reaction compartment is associated with acellular components of peripheral blood; and
the second reaction compartment is associated with tox pharmacology of the biological environments.

20. The non-transitory processor-readable medium of claim 15, wherein the code includes code to cause the processor to:
present the set of simulation results in an interactive graphical user interface that allows a user to adjust the plurality of parameters and the set of target criteria to analyze a correlation between the plurality of parameters and the set of target criteria.

21. The non-transitory processor-readable medium of claim 15, wherein the first subset of the plurality of parameters includes a nominal macro parameter from a set of nominal macro parameters, the set of nominal macro parameters including at least one of a time constant, a concentration of one or more states of the QSP model, or a thermodynamic constant.

22. An apparatus, comprising:
a memory; and
a processor operatively coupled to the memory, the processor configured to:
receive a user input that selects a quantitative system pharmacology (QSP) model from a plurality of QSP models, the QSP model having a plurality of parameters, the QSP model having a set of reaction compartments to characterize biological interactions between a therapeutic drug candidate and a target in biological environments;
generate a set of simulation results based on (1) the QSP model, (2) values of a set of macro parameters from the plurality of parameters, and (3) a set of target criteria,
the processor configured to generate the set of simulation includes scanning, from a first value of a set of values to a second value from the set of values, at least one parameter from a subset of the plurality of parameters;
determine if at least one value of the at least one parameter exists that satisfies the set of target criteria for the therapeutic drug candidate and the target for a therapeutic purpose; and
present the set of simulation results in an interactive graphical user interface that allows a user to adjust the plurality of parameters and the set of target criteria to analyze a correlation between the plurality of parameters and the set of target criteria.
